(19) United States
(12) Patent Application Publication
(10) Pub. No.: US 2022/0370301 A1
(43) Pub. Date: Nov. 24, 2022

(54) SKIN CLEANSING ARTICLE INCLUDING WATER-DISPERSIBLE AND/OR WATER-SOLUBLE CORE SUBSTRATE

(71) Applicant: MONOSOL, LLC, Merrillville, IN (US)

(72) Inventors: JONATHON D. KNIGHT, MERRILLVILLE, IN (US); VICTORIA BRIDEWELL, MERRILLVILLE, IN (US); JULIE S. PRATEL, MERRILLVILLE, IN (US); RICHARD GOETZ, MERRILLVILLE, IN (US); RYAN SLOPEK, MERRILLVILLE, IN (US)

(73) Assignee: MONOSOL, LLC, Merrillville, IN (US)

(21) Appl. No.: 17/737,969

(22) Filed: May 5, 2022

Related U.S. Application Data
(60) Provisional application No. 63/185,725, filed on May 7, 2021.

(51) Int. Cl. A61K 8/02; A61Q 19/10; A61K 8/81

(52) U.S. Cl. CPC A61K 8/0208; A61Q 19/10; A61K 8/8129; A61K 2800/56; A61K 2800/805

(57) ABSTRACT

A skin cleansing article configured to deliver cosmetics or dermal therapies to a user's skin and a method of making the same are provided. For example, the skin cleansing article includes a core substrate comprising a resin. The core substrate has a first region containing a first active cleansing formulation and a second region containing a second active cleansing formulation. The skin cleansing article is substantially dry or solid and is water-dispersible or water-soluble. When the water-soluble core substrate is contacted with water having a temperature greater than 10° C. for a period of time, the core substrate is dispersible or soluble according to Testing Method MSTM-205 to release the first active cleansing formulation and/or the second active cleansing formulation.

### CROSS-REFERENCE TO RELATED APPLICATIONS

This application claims the benefit of U.S. Provisional Application No. 63/185,725, filed May 7, 2021, which application is expressly incorporated by reference herein in its entirety.

### FIELD

The present disclosure relates generally to water-dispersible and/or water-soluble skin cleansing articles including water-dispersible or water-soluble core constructions. More particularly, the disclosure relates to water-dispersible and/or water-soluble skin cleansing articles including water-dispersible and/or water-soluble substrates, e.g., nonwoven, configured to contain an active cleansing formulation.

### BACKGROUND

Facial masks are typically placed on a user's face to apply an active skin agent. Conventional facial masks made of paper-based substrates are typically unpleasant to the touch and, because of the rigid or inflexible construction of the facial mask, do not intimately contact the user's face in order to maintain contact between the active skin agent and the user's skin. Further, as conventional facial masks become wet during use, these conventional facial masks may lose the structure and integrity necessary to maintain the facial mask properly positioned on the user's face and properly deliver the active skin agents to a predetermined location on the user's face. As a result, the active skin agents may migrate or move from the facial mask and into the user's eyes, nostrils, and/or mouth, resulting in unpleasant or undesirable effects, e.g., caustic or acidic ingredients may flow into the user's eyes, nostrils, and/or mouth causing cutaneous and/or membrane irritation and/or pain.

Further, conventional facial masks are many times stored in individual secondary packaging made of a plastic, foil, or composite material required to maintain the facial masks dry during shipping and/or storage. A moisture barrier layer, such as a moisture barrier film, coupled to a surface of the facial mask or enclosing the facial mask may also be required to maintain the structure and integrity of the facial mask, as well as the moisture content of the facial mask prior to use. This secondary packaging is generally not environmentally-friendly, compostable, recyclable, or biodegradable.

Thus, there exists a need in the art for skin cleansing articles having a construction that is easily manufacturable and that maintains its structure and integrity to maintain the facial mask properly positioned on the user's face with one or more regions of the facial mask aligned with a respective location on the user's face. Additionally, there exists a need in the art for a facial mask containing skin cleansing formulations that is easily applied to and easily removed from the user's face. Further, there exists a need in the art for a facial mask that significantly reduces the need for secondary packaging during shipping and storage prior to use of the facial mask.

### BRIEF DESCRIPTION OF THE DRAWINGS

FIG. 1 is a schematic plan view of an example water-dispersible skin cleansing article in the form of a facial mask and containing one or more active cleansing formulations in one or more respective regions of the facial mask, according to example embodiments;

FIG. 2 is a schematic sectional view of the example water-dispersible skin cleansing article taken along section line A-A, as shown in FIG. 1, according to example embodiments;

FIG. 3 is a schematic sectional view of another example water-dispersible skin cleansing article, according to example embodiments;

FIG. 4 is a perspective view of an example secondary packaging suitable for storing a plurality of water-dispersible or water-soluble skin cleansing articles, according to example embodiments;

FIG. 5 illustrates an example method for making a water-dispersible skin cleansing article, according to example embodiments;

FIG. 6 shows solubility results (at 23° C.) for example samples including a core substrate comprising at least one nonwoven layer or sheet having a plurality of fibers, which include a first type of fiber (“F1”) comprising a polyvinyl alcohol copolymer having a degree of hydrolysis of 88% and a second type of fiber (“F2”) comprising a polyvinyl alcohol copolymer having a degree of hydrolysis of 96%;

FIG. 7 shows solubility results (at 23° C.) for the example samples including the core substrates in FIG. 6 including an active cleansing formulation;

FIG. 8 shows solubility results (at 40° C.) for example core substrates comprising at least one nonwoven layer or sheet having a plurality of fibers, which include a first type of fiber (“F1”) comprising a polyvinyl alcohol copolymer having a degree of hydrolysis of 88% and a second type of fiber (“F2”) comprising a polyvinyl alcohol copolymer having a degree of hydrolysis of 96%;

FIG. 9 shows solubility results (at 40° C.) for the example samples including the core substrates in FIG. 8 including an active cleansing formulation; and

FIG. 10 shows Fourier transform infrared spectroscopy (FI-IR) curves illustrating transfer of an active cleansing formulation from a water-soluble nonwoven layer or sheet as the core substrate to a surface of a separate object made of polyester.

### DETAILED DESCRIPTION

In example embodiments described herein, water-dispersible and/or water-soluble skin cleansing articles include one or more water-dispersible substrates and/or one or more water-soluble core substrates, e.g., one or more water-dispersible nonwoven substrates and/or one or more water-soluble nonwoven substrates, having precision dosing to deliver active cleansing formulations, e.g., one or more cleansing agents, for delivering cosmetics and/or one or more dermal therapies to a user's skin. In example embodiments, the water-soluble skin cleansing article includes a water-soluble core substrate comprising a water-soluble resin. The water-soluble core substrate has one or more areas or regions configured to contain one or more active cleansing formulations, e.g., a cosmetic or dermal therapy formulation. For example, the water-soluble core substrate may have a first region containing a first active cleansing formulation and a second region containing a second active cleansing formulation, the same or different than the first active cleansing formulation. When the water-soluble core substrate is contacted with water having a temperature greater than 10° C., or having a temperature between 30° C. to 40° C. for a time of period from 30 seconds to 300 seconds (or 30 seconds to 600 seconds or 30 seconds to 900 seconds), the water-soluble core substrate is soluble to release at least one of the one or more active cleansing formulations, e.g., at least one of the first active cleansing formulation or the second active cleansing formulation. In example embodiments, the water-dispersible core substrate is cold water-dispersible or the water-soluble core substrate is cold water-soluble and tends to close skin pores, which may be advantageous for refining skin pores and the skin surface during an exfoliation process, for example. Alternatively, the water-dispersible core substrate is warm water-dispersible or the water-soluble substrate is warm water-soluble and tends to open skin pores, which may be advantageous for delivering an active cleansing formulation into the pores and the skin during an acne treating process, for example. In example embodiments, the water-dispersible skin cleansing article is initially dry, i.e., dry during storage and before use. In example embodiments, the nonwoven substrate transitions into a hydrogel as the water-dispersible core substrate is contacted with water over a suitable amount of time, e.g., for 300 seconds, at the desired temperature. In certain example embodiments, a film layer is created as the water-soluble core substrate dissolves to facilitate removal of any residual components of the water-soluble skin cleansing article after use. In certain embodiments, the water-dispersible or water-soluble skin cleansing article includes a film layer, e.g., a water-dispersible or water-soluble film, coupled to the water-dispersible or water-soluble core substrate. The water-dispersible or water-soluble film may include additional active cleansing formulations for delivery to the user's skin and/or may be used to facilitate removal of any residual components of the water-dispersible or water-soluble skin cleansing article after use.

In example embodiments, the skin cleansing article is configured to be at least water-dispersible or water-soluble upon contact with water for a period of time. The water-dispersible or water-soluble skin cleansing article as described herein is initially provided in a substantially dry or solid state, and water is added or applied to activate the skin cleansing article before or during use. Before the water is added or applied, the skin cleansing article in such a substantially dry or substantially solid state may contain no moisture or solvent, or less than 10 wt. % (such as less than 5 wt. %) of moisture or solvent. In example embodiments, the terms “substantially dry,” “substantially solid,” “dry,” or “solid” may refer to the skin cleansing article containing no moisture or solvent, or less than 10 wt. % (such as less than 5 wt. %) of moisture or solvent.

In example embodiments, the core substrate comprises a resin (i.e., a polymer), and may be water-dispersible or water-soluble. For example, the core substrate comprises at least one nonwoven substrate, which includes a plurality of fibers comprising a resin selected from at least one of a water-dispersible resin or a water-soluble resin. In addition to a nonwoven substrate, the substrate may also be a foam substrate or a film substrate. The resin can be any suitable polymer or may include one or more such polymers. For example, in example embodiments the resin is a polymer comprising a vinyl alcohol moiety. A “polymer comprising a vinyl alcohol moiety” or a “PVOH polymer” comprises a polyvinyl alcohol (PVOH) homopolymer, a polyvinyl alcohol (PVOH) copolymer, or a combination thereof. For example, the polyvinyl alcohol copolymer is a copolymer of vinyl acetate and vinyl alcohol in some embodiments. Such a polyvinyl alcohol copolymer may be an anionically modified copolymer, which may be a copolymer of vinyl acetate and vinyl alcohol further comprising additional groups such as a carboxylate, a sulfonate, or combinations thereof. Such a polymer comprising at least one of a vinyl acetate moiety or a vinyl alcohol moiety may also include an additional polymer, for example, in a blend. In example embodiments, a water-dispersible skin cleansing article is configured to deliver cosmetics or dermal therapies to a user's skin. The water-dispersible skin cleansing article includes a water-dispersible core substrate comprising a water-dispersible resin. The water-dispersible core substrate includes a first region containing a first active cleansing formulation and a second region containing a second active cleansing formulation, wherein, when the water-dispersible core substrate is contacted with water having a first temperature, e.g., water having a temperature of 10° C. or water having a temperature not greater than 40° C., the water-dispersible core substrate is activated to release the first active cleansing formulation and/or the second active cleansing formulation from the water-dispersible core substrate to deliver the first active cleansing formulation and/or the second active cleansing formulation to the user's skin, and, when the water-dispersible core substrate is contacted with water having a second temperature equal to or greater than the first temperature, e.g., water having a temperature equal to or greater than 40° C. for a time of period from 30 seconds to 300 seconds (or 30 seconds to 600 seconds or 30 seconds to 900 seconds), the water-dispersible core substrate is dispersible according to MSTM-205.

In other example embodiments, a water-soluble skin cleansing article is configured to deliver cosmetics or dermal therapies to a user's skin. The water-soluble skin cleansing article includes a water-soluble core substrate comprising a water-soluble resin. The water-soluble core substrate includes a first region containing a first active cleansing formulation and a second region containing a second active cleansing formulation, wherein, when the water-soluble core substrate is contacted with water having a first temperature, e.g., water having a temperature of 10° C. or water having a temperature not greater than 40° C., the water-soluble core substrate is activated to release the first active cleansing formulation and/or the second active cleansing formulation from the water-soluble core substrate to deliver the first active cleansing formulation and/or the second active cleansing formulation to the user's skin, and, when the water-soluble core substrate is contacted with water having a second temperature equal to or greater than the first temperature, e.g., water having a temperature equal to or greater than 40° C. for a time of period from 30 seconds to 300 seconds (or 30 seconds to 600 seconds or 30 seconds 900 seconds), the water-soluble core substrate is soluble according to MSTM-205.

Although the water-dispersible or water-soluble skin cleansing articles are described herein as a water-dispersible or water-soluble nonwoven substrate in the form of a facial mask configured to contain one or more active cleansing formulations in one or more areas or regions of the facial mask to deliver, e.g., release, the active cleansing formulation(s) to a desired location on the skin of the user's face, the water-dispersible or water-soluble skin cleansing articles as described herein are suitable in other example embodiments for delivering active cleansing formulations or other skin wellness formulations, for example, to other locations on the skin of the user's body. Further, the water-dispersible or water-soluble skin cleansing articles may take a form other than that of a facial mask including, without limitation, a wipe, a sheet, a pad, a sachet, or a strip, for example.

In example embodiments, the water-dispersible or water-soluble skin cleansing article is in the form of a water-dispersible or water-soluble facial mask made of a suitable water-dispersible or water-soluble core substrate, such as a water-dispersible nonwoven substrate or a water-soluble nonwoven substrate. Prior to use, the water-dispersible or water-soluble nonwoven substrate is substantially planar but formable to a contour of a user's body, e.g., formable to the contours of the skin surface of the user's face, once wetted with water before or during use. The water-dispersible or water-soluble nonwoven substrate includes openings for alignment with the user's eyes, nose, and mouth, respectively, to facilitate proper positioning of the facial mask on the user's face. In certain embodiments, a first active cleansing formulation, e.g., to treat wrinkles, is contained on or within a first region of the water-dispersible or water-soluble nonwoven substrate positioned with respect to a user's eye, e.g., to contact the user's skin around the respective eye and/or under the respective eye. Similarly, a second region of the water-dispersible or water-soluble nonwoven substrate may be positioned with respect to the user's forehead, e.g., to contact the user's skin on the user's forehead and/or bridge of the user's nose, and may contain the first active cleansing formulation, e.g., to treat wrinkles, and/or a second cleansing formulation, e.g., to treat acne. Additionally, or alternatively, a third region of the water-dispersible or water-soluble nonwoven substrate may positioned with respect to one or both of the user's cheeks or the user's chin, e.g., to contact the user's skin around the user's cheekbones and/or chin, and may contain the second active cleansing formulation, e.g., to treat acne, and/or a different active cleansing formulation to provide additional skin wellness formulations to the user's skin. In example embodiments, each of the first region, the second region, and the third region form at least a portion of the facial mask. In certain embodiments, one or more of the first region, the second region, or the third region may be separated from other regions of the facial mask before or during use.

In example embodiments, because of its high hydroscopic nature, the water-dispersible or water-soluble core substrate exists as a gel-like formulation during use to provide high hydration and a pleasing, soft effect. Additionally, the gel-like formulation effectively maintains the structure and proper positioning of the facial mask on the user's face, while maintaining the active cleansing formulation in proper position to contact the desired location on the user's face during use. Further, in example embodiments wherein the water-soluble core substrate is made of PVOH resin, the chemistry of the water-soluble core substrate and, specifically, the presence of PVOH, provides an emollient effect or benefit to the skin cleansing process.

In example embodiments, when the water-soluble core substrate is contacted with water having a temperature greater than 10° C., or having a temperature between 30° C. and 40° C., the water-soluble core substrate is soluble to release the active cleansing formulation. In example embodiments, the water-soluble core substrate includes a water-soluble polymer, such as polyvinyl alcohol (PVOH) copolymer and/or starch derivatives, for example, or blends thereof with otherwise water-dispersible polymers that have a high degree of biodegradation activity or can be composted or recycled.

In example embodiments, the water-soluble core substrate is a water-soluble nonwoven substrate made of a PVOH resin, such as a PVOH polymer. During use, the water-soluble nonwoven substrate dissolves into a gel-like substrate that facilitates maintaining the structure and integrity of the nonwoven substrate to maintain the facial mask properly positioned on the user's face, while providing a soft, pleasing touch. In example embodiments, a degree of hydrolysis of the PVOH copolymer and/or a stretching or drawing of the fibers, for example, can be adjusted to effect swelling and adsorption of the fibers to enhance the gel-like substrate.

The water-dispersible skin cleansing article and, more specifically, in example embodiments, the water-dispersible or water-soluble core substrate, is configured to contain one or more active cleansing formulations to deliver cosmetics or dermal therapies to a user's skin. As an example, the active cleansing formulation may include, without limitation, hyaluronic acid, aloe, chamomile extract, lactic acid, citric acid, hydrolyzed collagen, poly saccharides, peptides, surfactants, such as surfactants made from poly saccharides, or foaming agents, or any suitable combination thereof. Other suitable active cleansing formulations may include ceramides, glycolic acid and other alpha-hydroxy acids, amino acids, peptides, activated carbon, chemical and physical sunscreen ingredients, minerals (e.g., Zn), avobenzone, etc., antioxidants, energizers, such as caffeine, ginsing, taurine, etc., retinol, retinoic acid, Niacinamide, salicyclic acid, lactic acid, and/or aseliaic acid. In example embodiments, the active cleansing formulation is disposed on or coats one or more surfaces of the water-soluble core substrate or is embedded in and/or adhered to the water-soluble core substrate. The water-soluble core substrate may include a single layer, for example, a single layer nonwoven core substrate, or may include a plurality of layers, for example, a sheet of nonwoven core substrate folded in a serpentine arrangement or cut and plied to form layers with the active cleansing formulation disposed between one or more layers, e.g., adjacent layers, of the water-soluble nonwoven core substrate, for example.

In example embodiments, the water-soluble core substrate contains an active cleansing formulation, wherein upon contact with water having a suitable temperature, the water-soluble core substrate exhibits a degree of shrinkage of 0.5% to 65%. In example embodiments, when the core substrate is contacted with water having a temperature as low as 5° C. to 10° C., the core substrate is dispersible, i.e., disintegrates, to release the active cleansing formulation. In example embodiments, when the core substrate is contacted with water having a temperature greater than 40° C., the water-soluble core substrate is soluble, i.e., dissolves, to release the active cleansing formulation.

As used herein and unless specified otherwise, the term “water-dispersible” refers to any nonwoven substrate (or nonwoven web), foam substrate, film, or laminate wherein upon submersion in water at a specified temperature, the nonwoven substrate, foam substrate, film, or laminate physically disassociates into smaller constituent pieces. The smaller pieces may or may not be visible to the naked eye, may or may not remain suspended in the water, and may or may not ultimately dissolve. In example embodiments, such a nonwoven substrate (or nonwoven web), foam substrate, film, or laminate has a disintegration time of 900 seconds or less, or particularly, 600 seconds or less, or more particularly, 300 seconds or less at a specified temperature as determined according to MSTM-205 as set forth herein. In example embodiments wherein a dispersion temperature is not specified, the nonwoven substrate, foam substrate, film, or laminate will disintegrate in 300 seconds or less at a temperature of about 100° C. or less, according to MSTM-205. The disintegration time optionally can be 200 seconds or more, 100 seconds or more, 60 seconds or more, or 30 seconds or more at a temperature of about 80° C., about 70° C., about 60° C., about 50° C., about 40° C., about 20° C., or about 10° C., according to MSTM-205. In alternative example embodiments wherein a dispersion temperature is not specified, the nonwoven substrate, foam substrate, film, or laminate will disintegrate in 300 seconds or less at a temperature of about 100° C. or less, according to MSTM-205. The disintegration time optionally can be 200 seconds or less, 100 seconds or less, 60 seconds or less, or 30 seconds or less at a temperature of about 80° C., about 70° C., about 60° C., about 50° C., about 40° C., about 20° C., or about 10° C., according to MSTM-205. For example, such dispersion parameters can be characteristic of a nonwoven substrate, foam substrate, film, or laminate structure having a thickness of 6 millimeters (mm or mil) (about 152 microns (μm)). As described herein, in example embodiments, the disintegration time of the nonwoven substrate, foam substrate, film, or laminate may be greater than a minimum limit, such as 30 seconds, such that a resulting article, e.g., a facial mask, can be properly applied to a user's face, for example, and upon contacting the article with water, there is a suitable amount of time during application for the article to work as intended, e.g., deliver one or more active cleansing formulations to the user's skin providing the desired application benefits. In example embodiments, the nonwoven substrate (or nonwoven web), foam substrate, film, or laminate may have a disintegration time within a suitable range, for example, between 30 seconds and 900 seconds, between 30 seconds and 600 seconds, between 30 seconds and 300 seconds, between 60 seconds and 900 seconds, between 60 seconds and 600 seconds, or between 60 seconds and 300 seconds at a temperature of about 80° C., about 70° C., about 60° C., about 50° C., about 40° C., about 20° C., or about 10° C. according to MSTM-205, for example.

As used herein and unless specified otherwise, the term “water-soluble” refers to any nonwoven substrate (or nonwoven web), foam substrate, film, or laminate having a dissolution time, in example embodiments, of 900 seconds or less, or particularly, 600 seconds or less, or more particularly, 300 seconds or less at a specified temperature as determined according to MSTM-205 as set forth herein. Dissolution time may be at least partially dependent on the one or more active cleansing formulations used in the substrate, film, laminate, or article and/or the desired application process, as described herein. Additionally, in example embodiments, the nonwoven substrate (or nonwoven web), foam substrate, film, or laminate may have a dissolution time within a suitable range of dissolution times, for example, between 30 seconds and 900 seconds, between 30 seconds and 600 seconds, between 30 seconds and 300 seconds, between 60 seconds and 900 seconds, between 60 seconds and 600 seconds, or between 60 seconds and 300 seconds at a temperature of about 80° C., about 70° C., about 60° C., about 50° C., about 40° C., about 20° C., or about 10° C. according to MSTM-205, for example. In example embodiments, the dissolution time of the nonwoven substrate, foam substrate, film, or laminate optionally can be 900 seconds or less, 600 seconds or less, 200 seconds or less, 100 seconds or less, 60 seconds or less, or 30 seconds or less at a temperature of about 80° C., about 70° C., about 60° C., about 50° C., about 40° C., about 20° C., or about 10° C. according to MSTM-205. As described herein, in example embodiments, the dissolution time of the nonwoven substrate, foam substrate, film, or laminate may be greater than a minimum limit, such as 30 seconds, such that a resulting article, e.g., a facial mask, can be properly applied to a user's face, for example, and upon contacting the article with water, there is a suitable amount of time during application for the article to work as intended, e.g., deliver one or more active cleansing formulations to the user's skin providing the desired application benefits. In example embodiments wherein the dissolution temperature is not specified, the water-soluble nonwoven substrate, foam substrate, film, or laminate has a dissolution time of 300 seconds or less at a temperature no greater than about 80° C. In example embodiments, “water-soluble nonwoven substrate” or “water-soluble nonwoven web” means that at a thickness of 1.5 mil (about 38 μm), the nonwoven substrate dissolves in 300 seconds or less at a temperature no greater than 80° C. according to MSTM-205. For example, a 1.5 mil (about 38 μm) thick water-soluble nonwoven substrate can have a dissolution time of 300 seconds or less, 200 seconds or less, 100 seconds or less, or 60 seconds or less at a temperature of about 70° C., about 60° C., about 50° C., about 40° C., about 30° C., about 20° C., or about 10° C. according to MSTM-205.

As used herein and unless specified otherwise, the term “cold water-soluble” refers to any water-soluble nonwoven substrate, foam substrate, film, or laminate having a dissolution time of 300 seconds or less at a temperature in a range of about 10° C. to about 20° C. as determined according to MSTM-205. For example, the dissolution time of a cold water-soluble nonwoven substrate, foam substrate, film, or laminate optionally can be 200 seconds or less, 100 seconds or less, 60 seconds or less, or 30 seconds at a temperature in a range of about 10° C. to about 20° C. according to MSTM-205. In example embodiments, “cold water-soluble nonwoven substrate” or “cold water-soluble nonwoven web” means that at a thickness of 1.5 mil (about 38 μm), the nonwoven substrate dissolves in 300 seconds or less at a temperature not greater than 20° C. according to MSTM-205. For example, a 1.5 mil (about 38 μm) thick cold water-soluble nonwoven substrate can have a dissolution time of 300 seconds or less, 200 seconds or less, 100 seconds or less, 60 seconds or less, or 30 seconds or less at a temperature of about 20° C. or about 10° C. according to MSTM-205.

As used herein and unless specified otherwise, the term “hot water-soluble” refers to any water-soluble nonwoven substrate, foam substrate, film, or laminate having a dissolution time of 300 seconds or less at a temperature greater than about 20° C., for example in a range of about 21° C. to about 80° C., as determined according to MSTM-205. For example, the dissolution time of a hot water-soluble nonwoven substrate, foam substrate, film, or laminate optionally can be 200 seconds or less, 100 seconds or less, 60 seconds or less, or 30 seconds at a temperature greater than about 20° C. according to MSTM-205, for example, in a range of about 21° C. to about 80° C., about 25° C. to about 80° C., about 25° C. to about 60° C., about 30° C. to about 60° C., about 25° C. to about 45° C., about 30° C. to about 45° C., or about 25° C. to about 43° C., about 30° C. to about 43° C., about 25° C. to about 40° C., or about 30° C. to about 40° C. In example embodiments, “hot water-soluble nonwoven substrate” or “hot water-soluble nonwoven web” means that at a thickness of 1.5 mil (about 38 μm), the nonwoven substrate dissolves in 300 seconds or more at a temperature no less than about 21° C. according to MSTM-205. For example, a 1.5 mil (about 38 μm) thick water-soluble nonwoven substrate can have a dissolution time of 300 seconds or less, 200 seconds or less, 100 seconds or less, 60 seconds or less, or 30 seconds at a temperature of about 80° C., 70° C., about 60° C., about 50° C., about 40° C., about 30° C., about 25° C., or about 21° C. according to MSTM-205. In example embodiments, a hot water-soluble substrate, such as a “hot water-soluble nonwoven substrate” or a “hot water-soluble nonwoven web,” remains stable, e.g., does not dissolve, when contacted with water having a temperature less than its hot water-soluble temperature but is soluble, e.g., dissolves, when contacted with water having a temperature equal to its hot water-soluble temperature for a suitable dissolution time, e.g., in a range from 30 second to about 300 seconds. For example, in example embodiments, a hot water-soluble nonwoven substrate contacted with water having a temperature of 40° C. for 300 seconds or less, (or 600 seconds or less or 900 seconds or less), is soluble according to MSTM-205; however, the hot water-soluble nonwoven substrate is stable when contacted with water having a temperature less than 40° C. or contacted with water having a temperature of 40° C. for less than 300 seconds.

As used herein and unless specified otherwise, the term “nonwoven web” refers to a web or sheet comprising, consisting of, or consisting essentially of fibers arranged (e.g., by a carding process) and bonded to each other. Thus, the term “nonwoven web” can be considered short hand for nonwoven fiber-based webs. Further, as used herein, “nonwoven web” includes any structure including a nonwoven web or sheet, including, for example, a nonwoven web or sheet having a film laminated to a surface thereof. Methods of preparing nonwoven webs from fibers are well known in the art, for example, as described in Nonwoven Fabrics Handbook, prepared by Ian Butler, edited by Subhash Batra et al., Printing by Design, 1999, herein incorporated by reference in its entirety. As used herein and unless specified otherwise, the term “film” refers to a continuous film or sheet, e.g., prepared by a casting or extrusion process.

As used herein, a “plurality of fibers” can include a sole fiber type or can include two or more different fiber types. In example embodiments wherein the plurality of fibers comprise two or more different fiber types, each fiber type can be generally included in any amount, for example, from about 0.5 wt. % to about 99.5 wt. % of the total weight of the plurality of fibers. In example embodiments wherein the plurality of fibers consists of a sole fiber type, the plurality of fibers is substantially free of a second or more fiber types. A plurality of fiber is substantially free of a second or more fiber types when the plurality of fibers comprise less than about 0.5 wt. % of the second or more fiber types. In general, the difference between fiber types can be a difference in fiber length to diameter ratio (L/D), tenacity, shape, rigidness, elasticity, solubility, melting point, glass transition temperature (Tg), chemical composition, color, or a combination thereof.

As used herein, the terms “resin(s)” and “polymer(s)” should be considered interchangeable. In certain embodiments, the terms resin(s) and polymer(s), respectively, are used to refer to a polymer optionally combined with one or more additional polymers, and to a single type of polymer, e.g., a resin can comprise more than one polymer.

As used herein and unless specified otherwise, the terms “wt. %” and “wt %” are intended to refer to the composition of the identified element in “dry” (non-water) parts by weight of the entire water-soluble film, for example, including residual moisture in the water-soluble film, or parts by weight of the entire composition, depending on context.

As used herein and unless specified otherwise, the term “PHR” (“phr”) is intended to refer to the composition of the identified element in parts per one hundred parts water-soluble polymer resin(s) (whether PVOH or other polymer resins, unless specified otherwise) in the water-soluble nonwoven substrate, foam substrate, or film, or a solution used to make the water-soluble nonwoven substrate, foam substrate, or film.

As used herein and unless specified otherwise, the term “comprising” means that various components, ingredients, or steps can be conjointly employed in practicing the present disclosure. Accordingly, the term “comprising” encompasses the more restrictive terms “consisting essentially of” and “consisting of.” The present compositions can comprise, consist essentially of, or consist of any of the required and optional elements disclosed herein. The disclosure illustratively disclosed herein suitably may be practiced in the absence of any element or step which is not specifically disclosed herein.

When values are expressed as approximations, by use of the antecedent “about,” it will be understood that the particular value forms another embodiment. As used herein, “about X” (where X is a numerical value) in example embodiments refers to ±10% (for example, ±5%) of the recited value, inclusive.

The water-dispersible or water-soluble skin cleansing articles, the water-dispersible or water-soluble nonwoven materials, the water-dispersible or water-soluble foam materials, and the water-dispersible or water-soluble film materials, and related methods of making and using the water-dispersible or water-soluble skin cleansing articles, the water-dispersible or water-soluble nonwoven materials, the water-dispersible or water-soluble foam materials, and the water-dispersible or water-soluble film materials are contemplated to include embodiments including any combination of one or more of the additional optional elements, features, and steps further described below, unless stated otherwise.

In example embodiments, a water-dispersible skin cleansing article includes a water-soluble core substrate including a water-soluble resin. In example embodiments, the water-soluble core substrate includes one of more water-soluble nonwoven core substrates. The water-soluble core substrate contains an active cleansing formulation, wherein, when the water-soluble core substrate is contacted with water having a temperature greater than 10° C., or water having a temperature between 30° C. and 40° C., the water-soluble core substrate is soluble to release the active cleansing formulation. In example embodiments, the water-soluble core substrate is dispersible at 10° C. in 300 seconds, dissolvable at 20° C. in over 15 second but under 300 seconds, and dissolvable at 40° C. in over 15 seconds but under 300 seconds, and dissolvable at 80° C. in under 300 seconds.

In example embodiments, the active cleansing formulation is in the form of at least one of a solid, e.g., a powder or plurality of granules or particles, a gel, a liquid, or a slurry form, or any suitable combination thereof. In certain embodiments, the water-soluble core substrate is saturated with the active cleansing formulation. In other embodiments, the active cleansing formulation is embedded in, applied to, disposed on, coated on, and/or adhered to the water-soluble core substrate, e.g., the active cleansing formulation is disposed on a surface of the water-soluble core substrate. In example embodiments, the water-soluble core substrate is at least one of coated with the active cleansing formulation or impregnated with the active cleansing formulation. In example embodiments, the active cleansing formulation is present in the water-soluble core substrate, e.g., present in the fiber-forming composition, the foam-forming composition, or the film-forming composition.

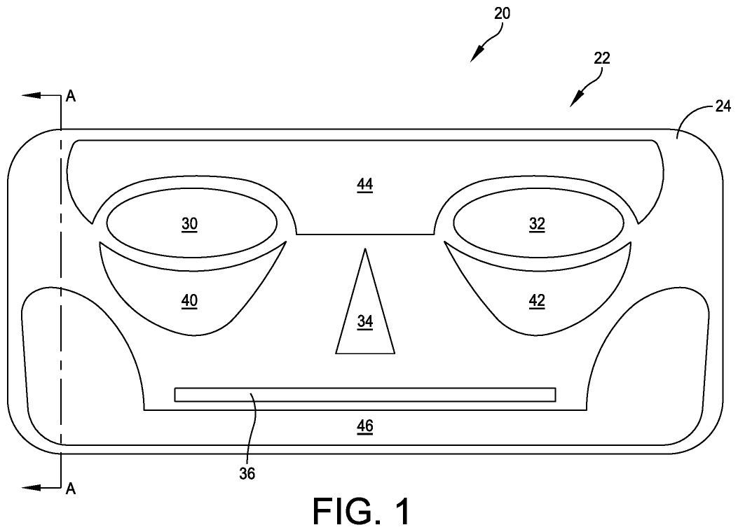

Referring to the Figures and, initially, to FIG. 1, in example embodiments, a water-soluble kin cleansing article 20 is in the form of a water-soluble facial mask 22 made of a suitable water-soluble core substrate, such as a water-soluble nonwoven substrate 24. While each of skin cleansing article 20, facial mask 22, the core substrate, and nonwoven substrate 24 are described as water-soluble in reference to FIGS. 1-4, each of skin cleansing article 20, facial mask 22, the core substrate, and nonwoven substrate 24 may include water-dispersible and/or water-soluble materials, e.g., a plurality of water-dispersible fibers, a plurality of water-soluble fibers, or blend of water-dispersible fibers and water-soluble fibers. Facial mask 22, e.g., water-soluble nonwoven substrate 24, has a first surface 26 configured to contact a user's skin and an opposing second surface 28. Prior to or during use, facial mask 22 is positioned on a user's face such that first surface 26 contacts a surface of skin on the user's face. In example embodiments, water-soluble nonwoven substrate 24 is substantially planar but, once wetted with water, for example, water-soluble nonwoven substrate 24 is formable to follow or form to a contour of a user's body, e.g., formable to the contours of the skin surface of the user's face. In other example embodiments, water-soluble nonwoven substrate 24 is nonplanar having surface contours configured or formed to follow a contour of the skin surface of the user's face, for example, around the user's eyes, along the user's forehead, and/or along the cheek bones and/or the chin of the user.

As shown in FIG. 1, facial mask 22, e.g., water-soluble nonwoven substrate 24, includes a plurality of openings for alignment with the user's eyes, nose, and mouth, respectively, to facilitate proper positioning of the facial mask on the user's face. For example, as shown in FIG. 1, nonwoven substrate 24 forms a first or right-eye opening 30 and a second or left-eye opening 32 for alignment with the user's right eye and left eye, respectively. Additionally, nonwoven substrate 24 forms a third opening 34 for alignment with the user's nose and a fourth opening 36 for alignment with the user's mouth. In example embodiments, nonwoven substrate 24 includes one or more regions, e.g., a plurality of regions, such as first region 40, second region 42, third region 44, and fourth region 46. Each region of the plurality of regions is configured to contain one or more active cleansing formulations 50.

In example embodiments, for example, a first active cleansing formulation, e.g., to treat wrinkles, is contained on or within first region 40 and/or contained on or within second region 42 of water-soluble nonwoven substrate 24 and positionable with respect to a user's eye, e.g., to contact the user's skin around the respective right eye or left eye and/or the user's skin under the respective eye. Additionally, or alternatively, third region 44 of water-soluble nonwoven substrate 24 is positionable with respect to the user's forehead, e.g., to contact the user's skin on the user's forehead and/or a bridge of the user's nose. Third region 44 is configured to contain the first active cleansing formulation, e.g., to treat wrinkles, and/or a second cleansing formulation, e.g., to treat acne, for example. Additionally, or alternatively, fourth region 46 of water-soluble nonwoven substrate 24 is positionable with respect to one or both of the user's cheeks and/or the user's chin, e.g., to contact the user's skin around the user's cheekbones and/or chin. Fourth region 46 is configured to contain the second active cleansing formulation, e.g., to treat acne, and/or a different active cleansing formulation to provide additional skin wellness formulations to the user's skin.

In example embodiments, each of first region 40, second region 42, third region 44, and fourth region 46 form at least a portion of water-soluble facial mask 22. In example embodiments, each of first region 40, second region 42, third region 44, and/or fourth region 46 have suitable dimensions for effectively delivering, e.g., releasing, active cleansing formulation 50 in desired locations on the user's skin. In certain embodiments, one or more of first region 40, second region 42, third region 44, or fourth region 46 may overlap adjacent regions. Further, one or more of first region 40, second region 42, third region 44, or fourth region 46 can be separated from water-soluble facial mask 22 before or during use.

In example embodiments, a skin cleansing article, such as a facial mask, a wipe, a sheet, a pad, a sachet, or a strip, for example, is configured to deliver cosmetics and/or dermal therapies to a user's skin. The skin cleansing article includes a first nonwoven substrate including a plurality of fibers comprising a water-soluble resin. The first nonwoven substrate has at least one first region with a first active cleansing formulation contained in the first region. A second nonwoven substrate is coupled to the first nonwoven substrate. The second nonwoven substrate includes a plurality of fibers comprising a water-dispersible resin and/or a water-soluble resin. The second nonwoven substrate has at least one second region with a second active cleansing formulation contained in the second region. In example embodiments, when the first nonwoven substrate is contacted with water having a temperature greater than 10° C. for 300 seconds or less (e.g., 30 second to 300 seconds), the first nonwoven substrate is soluble according to MSTM-205 to release the first active cleansing formulation from the first nonwoven substrate. In certain embodiments, the second nonwoven substrate includes a plurality of fibers comprising a water-dispersible resin, and, when the second nonwoven substrate is contacted with water having a temperature greater than 10° C. for 300 seconds or less (e.g., 30 second to 300 seconds), the second nonwoven substrate is dispersible according to MSTM-205 to release the second active cleansing formulation from the second nonwoven substrate. In certain embodiments, the second nonwoven substrate includes a plurality of fibers comprising a water-soluble resin, and, when the second nonwoven substrate is contacted with water having a temperature greater than 10° C. for 300 seconds or less (e.g., 30 second to 300 seconds), the second nonwoven substrate is soluble according to MSTM-205 to release the second active cleansing formulation from the second nonwoven substrate. In example embodiments, a water-soluble film, a water-dispersible film, and/or a biodegradable film is coupled to, e.g., laminated to, the first nonwoven substrate and/or the second nonwoven substrate.

In example embodiments, a skin cleansing article, such as a facial mask, a wipe, a sheet, a pad, a sachet, or a strip, for example, is configured to deliver cosmetics and/or dermal therapies to a user's skin. The skin cleansing article includes a first nonwoven substrate including a plurality of fibers comprising a water-dispersible resin. The first nonwoven substrate has at least one first region with a first active cleansing formulation contained in the first region. A second nonwoven substrate is coupled to the first nonwoven substrate. The second nonwoven substrate includes a plurality of fibers comprising a water-dispersible resin and/or a water-soluble resin. The second nonwoven substrate has a second region with a second active cleansing formulation contained in the second region, When the first nonwoven substrate is contacted with water having a temperature greater than 10° C. for 300 seconds or less (e.g., 30 second to 300 seconds, the first nonwoven substrate is dispersible according to MSTM-205 to release the first active cleansing formulation from the first nonwoven substrate. In certain embodiments, the second nonwoven substrate includes a plurality of fibers comprising a water-dispersible resin, and, when the second nonwoven substrate is contacted with water having a temperature greater than 10° C. for 300 seconds or less (e.g., 30 second to 300 seconds, the second nonwoven substrate is dispersible according to MSTM-205 to release the second active cleansing formulation from the second nonwoven substrate. In certain embodiments, the second nonwoven substrate includes a plurality of fibers comprising a water-soluble resin, and, when the second nonwoven substrate is contacted with water having a temperature greater than 10° C. for 300 seconds or less (e.g., 30 second to 300 seconds, the second nonwoven substrate is soluble according to MSTM-205 to release the second active cleansing formulation from the second nonwoven substrate. In example embodiments, a water-soluble film, a water-dispersible film, and/or a biodegradable film is coupled to, e.g., laminated to, the first nonwoven substrate and/or the second nonwoven substrate.

Referring further to FIGS. 1-3, water-soluble skin cleansing article 20 includes water-soluble nonwoven substrate 24 comprising a water-soluble resin. In example embodiments, water-soluble nonwoven substrate 24 includes any suitable fiber chemistry including, without limitation, PVOH polymer fibers or PVOH polymer fibers blended with up to 90 wt. % cellulose-type fibers. In alternative embodiments, the nonwoven substrate is made of water-dispersible fibers. In example embodiments, water-soluble nonwoven substrate 24 has a basis weight of 10 gsm (grams per square meter) to 120 gsm, and, more particularly, 15 gsm to 100 gsm, and, even more particularly, 30 gsm to 80 gsm, and, even more particularly, 30 gsm to 40 gsm; a fiber length of 10 millimeters (mm) to 100 mm; and a suitable fiber diameter of 5 microns to 100 microns. In other example embodiments, water-soluble nonwoven substrate 24 has any suitable basis weight, fiber length, and/or fiber diameter. For example, in example embodiments, the fiber diameter is less than 5 microns or greater than 100 microns. The fibers of water-soluble nonwoven substrate 24 may be created using any suitable methods including, without limitation, a carded and calendered process or any suitable process for making water-soluble nonwoven fibers. Further, the fibers of water-soluble nonwoven substrate 24 are bonded together using any suitable bonding process or method including, without limitation, a heat, thermal, chemical, water, and/or solution bonding method or any suitable bonding method known in the art of nonwoven fiber bonding. As described herein, water-soluble nonwoven substrate 24 may include any suitable number of layers or plies, for example, 1 layer or ply to 50 layers or plies, or more in certain embodiments. Water-soluble nonwoven substrate 24 may be porous or non-porous and cold water-soluble, warm water-soluble, or hot water-soluble. Water-soluble nonwoven substrate 24 may be formed using any suitable manufacturing process known in the nonwoven manufacturing art including, without limitation, a carded process. The construction of water-soluble substrate 22 may include, for example, folded layers or plies, stacked layers or plies, and/or rolled layers or plies.

In example embodiments, water-soluble nonwoven substrate 24 is configured to contain active cleansing formulation 50. When water-soluble nonwoven substrate 24 is contacted with water having a temperature greater than 20° C., or a temperature between 30° C. and 40° C., water-soluble nonwoven substrate 24 is soluble to release active cleansing formulation 50 and deliver active cleansing formulation 50 to a desired location on the user's face. Active cleansing formulation 50 may be in the form of a solid, e.g., a powder or a plurality of granules or particles, a gel, a liquid, or a slurry formulation, or any suitable combination of a solid, a gel, a liquid, or a slurry formulation, for example. In example embodiments, active cleansing formulation 50 is in any suitable phase including, for example, a solid phase, a liquid phase, a slurry phase (a liquid containing solids and multiple phases), or any suitable combination of phases. For example, active cleansing formulation 50 may include fine powder particles or granules, gels, one or more liquids, or a slurry, or multiple phases. In example embodiments, active cleansing formulation 50 includes, without limitation, one or more of the following: hyaluronic acid, aloe, chamomile extract, lactic acid, citric acid, hydrolyzed collagen, a poly saccharide, a peptide, a surfactant, or a foaming agent, detergents, surfactants, emulsifiers, chelants, enzymes, pH adjusters, builders, structurants, free fragrance, encapsulated fragrance, preservatives, solvent, minerals, and/or any ingredient suitable for including in a skin cleansing formulation, a skin wellness formulation, or a personal care formulation. In example embodiments, skin cleansing article 20 includes active cleansing formulation 50 having a mass of 0.5 gram (g) to 250 grams and, more particularly, 3.0 grams to 8.0 grams, and, even more particularly, for select ingredients, 0.1 g to 3.0 g and a volume of 1.0 milliliter (ml) to 250 ml. In example embodiments wherein active cleansing formulation 50 is a solid phase, the particles or granules may have a size of 1 micron to 100 microns, for example, or may be in tablet form.

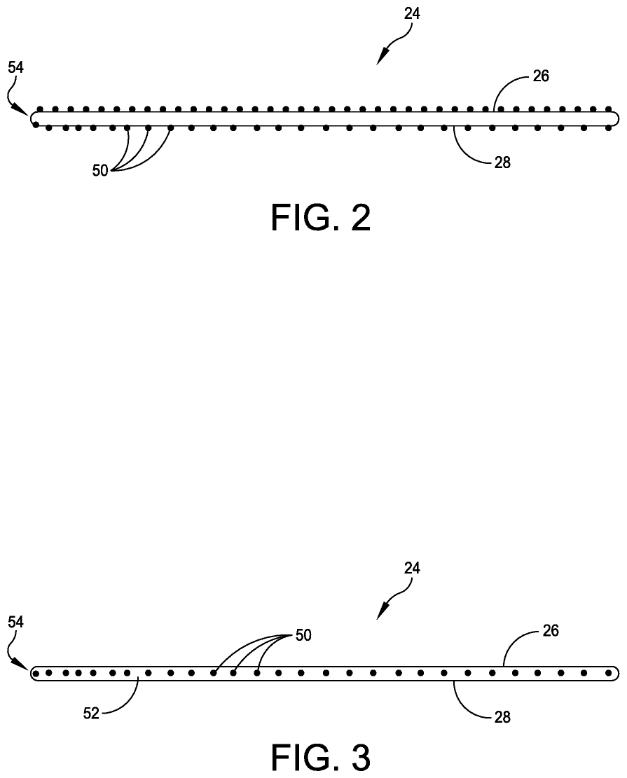

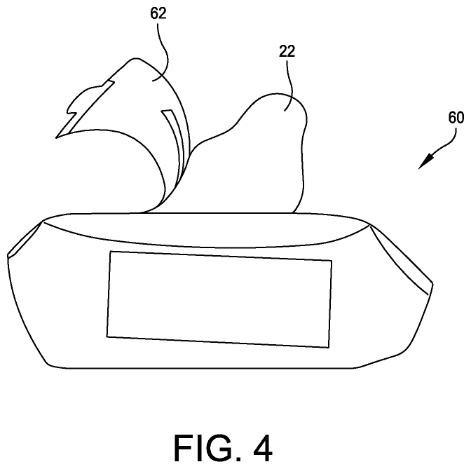

In example embodiments, active cleansing formulation 50 is contained in, on, or by water-soluble nonwoven substrate 24, for example, by saturating water-soluble nonwoven substrate 24 with active cleansing formulation 50, by disposing active cleansing formulation 50 on one or more surfaces, e.g., first surface 26 and/or second surface 28, of water-soluble nonwoven substrate 24, as shown in FIG. 2, by embedding active cleansing formulation 50 in a matrix 52 of water-soluble nonwoven substrate 24, as shown in FIG. 3, e.g., in one or more layers of water-soluble nonwoven substrate 24, and/or by disposing active cleansing formulation 50 between different layers, e.g., adjacent layers, of water-soluble nonwoven substrate 24, e.g., coating one or more surfaces of one or more layers, for example, with active cleansing formulation 50. Active cleansing formulation 50 may be impregnated in, adsorbed in, and/or adhered to or bonded to a surface of water-soluble nonwoven substrate 24, for example.

In example embodiments such as shown in FIG. 2, skin cleansing article 20 includes one or more layers of water-soluble nonwoven substrate 24 in the form of a nonwoven sheet 54 and active cleansing formulation 50 in a solid phase disposed on first surface 26 and/or opposing second surface 28 of water-soluble nonwoven substrate 24. In example embodiments, such as shown in FIG. 3, skin cleansing article 20 includes one or more layers of water-soluble nonwoven substrate 24 forming nonwoven sheet 54 containing active cleansing formulation 50 in a solid phase embedded within matrix 52 of water-soluble nonwoven substrate 24.

Referring further to FIGS. 1-3, in example embodiments, water-soluble nonwoven substrate 24 includes a plurality of fibers as described herein but not explicitly shown in FIGS. 1-3. In example embodiments, one or more fibers of the plurality of fibers is saturated with or impregnated with active cleansing formulation 50. Active cleansing formulation 50 may be embedded in one or more of fibers of the plurality of fibers or between one or more adjacent fibers of the plurality of fibers, or active cleansing formulation 50 may be disposed on, e.g., coated on, a surface of one or more fibers of the plurality of fibers.

As shown in FIG. 4, a sustainable packaging 60 made of a suitable recyclable material, such as cardboard, paper board, coated paper, barrier paper, repulpable packaging, recyclable plastic, or other paper-based materials, is configured to contain or store one or more facial masks 22, e.g., a plurality facial masks 22. Prior to use, the user opens tab 62 and removes one facial mask 22 from within packaging 60 for use. Tab 62 is then closed to seal packaging 60. In example embodiments, the facial mask is initially provided in a dry state and water is added to the facial mask before or during use.

In example embodiments, an example water-soluble skin cleansing article 20 is provided that is positionable on an area of a user's skin. For example, water-soluble skin cleansing article 20 in the form of facial mask 22 is positionable on the user's face to contact at least a portion of the skin surface of the user's face. For example, first opening 30 is aligned with and positioned about the user's right eye, second opening 32 is aligned with and positioned about the user's left eye. Additionally, third opening 34 is aligned with and positioned about the user's nose and fourth opening 36 is aligned with and positioned about the user's mouth. With facial mask 22 properly positioned on the user's face, one or more regions, e.g., a plurality of regions, such as first region 40, second region 42, third region 44, and fourth region 46 of facial mask 22, containing one or more active cleansing formulations 50, are properly positioned to contact respective regions of the skin surface of the user's face. For example, a first active cleansing formulation, e.g., to treat wrinkles, is contained on or within first region 40 and/or contained on or within second region 42 of water-soluble nonwoven substrate 24 and positioned with respect to a user's eye, e.g., to contact the user's skin around the respective right eye or left eye and/or the user's skin under the respective eye. Third region 44 of water-soluble nonwoven substrate 24 is positioned with respect to the user's forehead, e.g., to contact the user's skin on the user's forehead and/or a bridge of the user's nose. Third region 44 is configured to contain the first active cleansing formulation, e.g., to treat wrinkles, and/or a second cleansing formulation, e.g., to treat acne, for example. Fourth region 46 of water-soluble nonwoven substrate 24 is positioned with respect to one or both of the user's cheeks and/or the user's chin, e.g., to contact the user's skin around the user's cheekbones and/or chin. Fourth region 46 is configured to contain the second active cleansing formulation, e.g., to treat acne, and/or a different active cleansing formulation to provide additional skin wellness formulations to the user's skin. Any suitable skin care ingredient for providing various desired effects including, without limitation, hydrating, moisturizing, brightening, tightening, and/or pore cleansing, or a perfume or naturally scented essential oil or extract, may be included in the one or more active cleansing formulations in example embodiments. As water-soluble skin cleansing article 20, e.g., facial mask 22, is contacted with water having a temperature greater than 20° C., or water having a temperature between 30° C. and 40° C., water-soluble skin cleansing article 20 is soluble to release one or more active cleansing formulations 50 in one or more regions of facial mask 22. After use, any remaining portion of facial mask 22 is removed from the user's face and discarded. Alternatively, in example embodiments, the remaining portion of facial mask 22 and/or any remaining active skin formulation 50 forms a lather that the user can massage into his or her skin using his or her wet hands or fingers. In example embodiments, the user is able to use all of active skin formulation 50 and does not feel as if he or she is not utilizing the entire amount of active skin formulation 50. In certain example embodiments, two or more regions of the plurality of regions, such as first region 40, second region 42, third region 44, and/or fourth region 46 of facial mask 22, may also comprise an identical or substantially identical cleansing formulation 50.

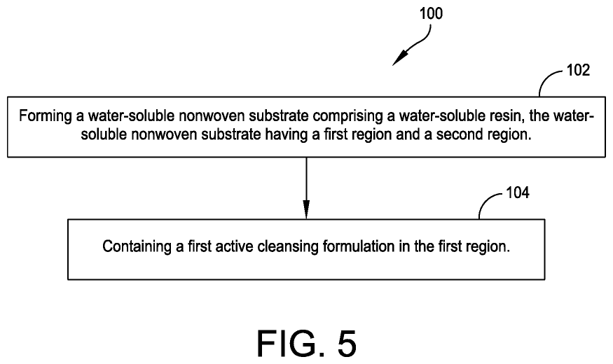

Referring now to FIG. 5, in example embodiments, an exemplary method 100 for making skin cleansing articles containing an active cleansing formulation includes steps 102, 104 and/or 106. At step 102, a water-soluble core substrate comprising a water-soluble resin is formed. In example embodiments, the water-soluble nonwoven substrate has a first region configured to contain a first active cleansing formulation and a second region configured to contain a second active cleansing formulation. At step 104, a first active cleansing formulation is contained in the first region. At step 106, and a second active cleansing formulation is contained in the second region. When the water-soluble nonwoven substrate is contacted with water having a temperature greater than 20° C., the water-soluble nonwoven substrate is soluble according to MSTM-205 to release at least one of the first active cleansing formulation and the second active cleansing formulation.

In example embodiments, step 102 of forming a water-soluble core substrate comprising a water-soluble resin includes forming one or more layers of a water-soluble nonwoven substrates. In example embodiments, the water-soluble core substrate, e.g., the water-soluble nonwoven substrate, is configured to contain the one or more active cleansing formulation, such as described herein. In example embodiments, active cleansing formulation 50 is contained in or by water-soluble nonwoven substrate 24, for example, by saturating water-soluble nonwoven substrate 24 with active cleansing formulation 50, by disposing active cleansing formulation 50 on one or more surfaces, e.g., a first surface 28 and/or a second surface 30, of water-soluble nonwoven substrate 24, as shown in FIG. 2, by embedding active cleansing formulation 50 in a matrix 52 of water-soluble nonwoven substrate 24, as shown in FIG. 3, e.g., in one or more layers of water-soluble nonwoven substrate 24, and/or by disposing active cleansing formulation 50 between different layers, e.g., adjacent layers, of water-soluble nonwoven substrate 24, e.g., coating one or more surfaces of one or more layers, for example, with active cleansing formulation 50. Active cleansing formulation 50 may be adsorbed in and/or adhered to or bonded to a surface of water-soluble nonwoven substrate 24, for example. When the water-soluble core substrate is contacted with water having a temperature greater than 20° C., or water having a temperature between 30° C. and 40° C., the water-soluble core substrate is soluble to release the active cleansing formulation.

### Water-Soluble Film and Fiber-Forming Materials

Water-soluble polymers for use in the water-soluble fibers, water-soluble nonwoven substrates, water-soluble foam substrates, and water-soluble films include, but are not limited to, a polyvinyl alcohol, polyacrylate, water-soluble acrylate copolymer, polyvinyl pyrrolidone, polyethyleneimine, pullulan, water-soluble natural polymers including, but not limited to, guar gum, gum Acacia, xanthan gum, carrageenan, and starch, water-soluble polymer derivatives including, but not limited to, modified starches, ethoxylated starch, and hydroxypropylated starch, copolymers of the forgoing and combinations of any of the foregoing. Other water-soluble polymers can include polyalkylene oxides, polyacrylamides, polyacrylic acids and salts thereof, celluloses, cellulose ethers, cellulose esters, cellulose amides, polyvinyl acetates, polycarboxylic acids and salts thereof, polyaminoacids, polyamides, gelatines, methylcelluloses, carboxymethylcelluloses and salts thereof, dextrins, ethylcelluloses, hydroxyethyl celluloses, hydroxypropyl methylcelluloses, maltodextrins, polymethacrylates, and combinations of any of the foregoing. Such water-soluble polymers, whether PVOH polymers or otherwise, are commercially available from a variety of sources.

In general, the fibers, foams, and films as described herein include polyvinyl alcohol. Polyvinyl alcohol is a synthetic polymer generally prepared by the alcoholysis, usually termed “hydrolysis” or “saponification,” of polyvinyl acetate. Fully hydrolyzed PVOH, where virtually all the acetate groups have been converted to alcohol groups, is a strongly hydrogen-bonded, highly crystalline polymer which dissolves only in hot water, e.g., water having a temperature greater than about 140° F. (about 60° C.). If a sufficient number of acetate groups are allowed to remain after the hydrolysis of polyvinyl acetate, that is the PVOH polymer is partially hydrolyzed, then the polymer is more weakly hydrogen-bonded, less crystalline, and is generally soluble in cold water, e.g., water having a temperature less than about 50° F. (about 10° C.). As such, the partially hydrolyzed polymer is a vinyl alcohol-vinyl acetate copolymer that is a PVOH copolymer, but is commonly referred to as PVOH.

In certain example embodiments, suitable examples of such a polymer include, without limitation, a polyvinyl alcohol homopolymer, a polyvinyl alcohol copolymer, a modified polyvinyl alcohol copolymer, and combinations thereof. For example, the polyvinyl alcohol copolymer is a copolymer of vinyl acetate and vinyl alcohol in certain embodiments. For example, in some embodiments, the modified polyvinyl alcohol copolymer comprises an anionically modified copolymer, which may be a copolymer of vinyl acetate and vinyl alcohol further comprising additional groups such as a carboxylate, a sulfonate, or combinations thereof. As such, the partially hydrolyzed polymer is a vinyl alcohol-vinyl acetate copolymer that is a PVOH copolymer, but is commonly referred to as “polyvinyl alcohol (PVOH)” or “PVOH polymer.” For brevity, the term “PVOH polymer” as used herein is understood to encompass a homopolymer, a copolymer, and a modified copolymer comprising vinyl alcohol moieties, for example, 50% or greater of vinyl alcohol moieties.

The fibers, foams, and/or films described herein can include one or more polyvinyl alcohol (PVOH) homopolymers, one or more polyvinyl alcohol copolymers, one or more modified polyvinyl alcohol copolymers, or a combination thereof. As used herein, the term “homopolymer” generally includes polymers having a single type of monomeric repeating unit (e.g., a polymeric chain consisting of or consisting essentially of a single monomeric repeating unit). For the particular case of PVOH, the term “PVOH polymer” further includes copolymers consisting of a distribution of vinyl alcohol monomer units and vinyl acetate monomer units, depending on the degree of hydrolysis (e.g., a polymeric chain consisting of or consisting essentially of vinyl alcohol and vinyl acetate monomer units). In the limiting case of 100% hydrolysis, a PVOH homopolymer can include a true homopolymer having only vinyl alcohol units. In some embodiments, the fibers, foams, and/or films of the disclosure include polyvinyl alcohol copolymers. In some embodiments, the fibers, foams, and/or films of the disclosure include cold-water soluble or hot water-soluble polyvinyl alcohol copolymers.

Unless expressly indicated otherwise, the term “degree of hydrolysis” is understood as a percentage (e.g., a molar percentage) of hydrolyzed moieties among all hydrolyzable moieties of an initial polymer. For example, for a polymer comprising at least one of a vinyl acetate moiety or a vinyl alcohol moiety, partial replacement of an ester group in vinyl acetate moieties with a hydroxyl group occurs during hydrolysis, and a vinyl acetate moiety becomes a vinyl alcohol moiety. The degree of hydrolysis of a polyvinyl acetate homopolymer is considered as zero, while the degree of hydrolysis of a polyvinyl alcohol homopolymer is considered as 100%. The degree of hydrolysis of a copolymer of vinyl acetate and vinyl alcohol is equal to a percentage of vinyl alcohol moieties among a total of vinyl acetate and vinyl alcohol moieties, and is considered between zero and 100%.

In some embodiments, the polyvinyl alcohol includes a modified polyvinyl alcohol, for example, a copolymer. The modified polyvinyl alcohol can include a co-polymer or higher polymer (e.g., ter-polymer) including one or more monomers in addition to the vinyl acetate/vinyl alcohol groups. Optionally, the modification is neutral, e.g., provided by an ethylene, propylene, N-vinylpyrrolidone or other non-charged monomer species. Optionally, the modification is a cationic modification, e.g., provided by a positively charged monomer species. Optionally, the modification is an anionic modification. Thus, in some embodiments, the polyvinyl alcohol includes an anionic modified polyvinyl alcohol.

An anionic modified polyvinyl alcohol can include a partially or fully hydrolyzed PVOH copolymer that includes an anionic monomer unit, a vinyl alcohol monomer unit, and optionally a vinyl acetate monomer unit (i.e., when not completely hydrolyzed). In some embodiments, the modified PVOH copolymer can include two or more types of anionic monomer units. General classes of anionic monomer units which can be used for the PVOH copolymer include the vinyl polymerization units corresponding to sulfonic acid vinyl monomers and their esters, monocarboxylic acid vinyl monomers, their esters and anhydrides, dicarboxylic monomers having a polymerizable double bond, their esters and anhydrides, and alkali metal salts of any of the foregoing. Examples of suitable anionic monomer units include the vinyl polymerization units corresponding to vinyl anionic monomers including vinyl acetic acid, maleic acid, monoalkyl maleate, dialkyl maleate, maleic anhydride, fumaric acid, monoalkyl fumarate, dialkyl fumarate, itaconic acid, monoalkyl itaconate, dialkyl itaconate, citraconic acid, monoalkyl citraconate, dialkyl citraconate, citraconic anhydride, mesaconic acid, monoalkyl mesaconate, dialkyl mesaconate, glutaconic acid, monoalkyl glutaconate, dialkyl glutaconate, alkyl acrylates, alkyl alkacrylates, vinyl sulfonic acid, allyl sulfonic acid, ethylene sulfonic acid, 2-acrylamido-1-methyl propane sulfonic acid, 2-acrylamide-2-methylpropanesulfonic acid, 2-methylacrylamido-2-methylpropanesulfonic acid (AMPS), 2-sulfoethyl acrylate, alkali metal salts of the foregoing (e.g., sodium, potassium, or other alkali metal salts), esters of the foregoing (e.g., methyl, ethyl, or other C₁-C₄ or C₆ alkyl esters), and combinations of the foregoing (e.g., multiple types of anionic monomers or equivalent forms of the same anionic monomer). In some embodiments, the modified PVOH copolymer can include two or more types of monomer units selected from neutral, anionic, and cationic monomer units.

The level of incorporation of the one or more anionic monomer units in the PVOH copolymers is not particularly limited. In certain embodiments, the one or more anionic monomer units are present in the PVOH copolymer in an amount in a range of about 1 mol. % or 2 mol. % to about 6 mol. % or 10 mol. % (e.g., at least 1.0, 1.5, 2.0, 2.5, 3.0, 3.5, or 4.0 mol. % and/or up to about 3.0, 4.0, 4.5, 5.0, 6.0, 8.0, or 10 mol. % in various embodiments).

Polyvinyl alcohols can be subject to changes in solubility characteristics. The acetate group in the co-poly(vinyl acetate vinyl alcohol) polymer (PVOH copolymer) is known by those skilled in the art to be hydrolysable by either acid or alkaline hydrolysis. As the degree of hydrolysis increases, a polymer composition made from the PVOH copolymer will have increased mechanical strength but reduced solubility at lower temperatures (e.g., requiring hot water temperatures for complete dissolution). Accordingly, exposure of a PVOH copolymer to an alkaline environment (e.g., resulting from a laundry bleaching additive) can transform the polymer from one which dissolves rapidly and entirely in a given aqueous environment (e.g., a cold-water medium) to one which dissolves slowly and/or incompletely in the aqueous environment, potentially resulting in undissolved polymeric residue.

The degree of hydrolysis (DH) of the PVOH homopolymers and PVOH copolymers (including modified PVOH copolymers) included in the water-soluble fibers, foams, and films of the present disclosure can be in a range of about 75% to about 99.9% (e.g., about 79% to about 92%, about 75% to about 89%, about 80% to about 90%, about 88% to 92%, about 86.5% to about 89%, or about 88%, 90% or 92% such as for cold-water-soluble compositions; about 90% to about 99.9%, about 90% to about 99% about 92% to about 99%, about 95% to about 99%, about 98% to about 99%, about 98% to about 99.9%, about 96%, about 98%, about 99%, or greater than 99%). As the degree of hydrolysis is reduced, a fiber, foam, or film made from the polymer will have reduced mechanical strength but faster solubility at temperatures below about 20° C. As the degree of hydrolysis increases, a fiber, foam, or film made from the polymer will tend to be mechanically stronger and the thermoformability will tend to decrease. The degree of hydrolysis of the PVOH can be chosen such that the water-solubility of the polymer is temperature dependent, and thus the solubility of a film, foam, or fiber made from the polymer and additional ingredients is also influenced. In certain embodiments, the film, foam, and/or fibers are cold water-soluble. For a co-poly(vinyl acetate vinyl alcohol) polymer that does not include any other monomers (e.g., a copolymer not copolymerized with an anionic monomer) a cold water-soluble fiber, foam, or film, soluble in water at a temperature of less than 10° C., can include PVOH with a degree of hydrolysis in a range of about 75% to about 90%, about 75% to about 89%, or in a range of about 80% to about 90%, or in a range of about 85% to about 90%. In another embodiment, the fiber, foam, or film is hot water-soluble. For a co-poly(vinyl acetate vinyl alcohol) polymer that does not include any other monomers (e.g., a copolymer not copolymerized with an anionic monomer) a hot water-soluble fiber, foam, or film that is soluble in water at a temperature of at least about 60° C., can include PVOH with a degree of hydrolysis of at least about 98%. In example embodiments, one of more of the plurality of fibers comprise a polyvinyl alcohol polymer having a degree of hydrolysis in a range of about 75% to about 99.9%. In example embodiments, one or more of the plurality of fibers comprise a polyvinyl alcohol polymer having a degree of hydrolysis in a range of about 75% to about 98%. In example embodiments, one of more of the plurality of fiber comprises a polyvinyl alcohol polymer having a degree of hydrolysis in a range of about 75% to about 89%. In example embodiments, one of more of the plurality of fiber comprises a polyvinyl alcohol polymer having a degree of hydrolysis in a range of about 90% to about 99.9%. In example embodiments, the water-soluble film comprises a polyvinyl alcohol copolymer or a modified PVOH copolymer having a degree of hydrolysis in a range of about 75% to about 99.9%. In example embodiments, the water-soluble film comprises a polyvinyl alcohol homopolymer or a polyvinyl alcohol copolymer having a degree of hydrolysis in a range of about 75% to about 98%.

The degree of hydrolysis of a polymer blend can also be characterized by the arithmetic weighted, average degree of hydrolysis (H̅°̅). For example, H̅°̅ for a PVOH polymer that includes two or more PVOH polymers is calculated by the formula H̅°̅=ρ(Wᵢ·Hᵢ) where Wᵢ is the molar percentage of the respective PVOH polymer and Hᵢ is the respective degrees of hydrolysis. When a polymer is referred to as having (or not having) a specific degree of hydrolysis, the polymer can be a single polyvinyl alcohol polymer having the specified degree of hydrolysis or a blend of polyvinyl alcohol polymers having an average degree of hydrolysis as specified.

The viscosity of a PVOH polymer (μ) is determined by measuring a freshly made solution using a Brookfield LV type viscometer with UL adapter as described in British Standard EN ISO 15023-2:2006 Annex E Brookfield Test method. It is international practice to state the viscosity of 4% aqueous polyvinyl alcohol solutions at 20° C. All viscosities specified herein in Centipoise (cP) should be understood to refer to the viscosity of 4% aqueous polyvinyl alcohol solution at 20° C., unless specified otherwise. Similarly, when a polymer is described as having (or not having) a particular viscosity, unless specified otherwise, it is intended that the specified viscosity is the average viscosity for the polymer, which inherently has a corresponding molecular weight distribution, i.e., the weighted natural log average viscosity. It is well known in the art that the viscosity of PVOH polymers is correlated with the weight average molecular weight (M̅w) of the PVOH polymer, and often the viscosity is used as a proxy for the M̅w.

In example embodiments, the PVOH resin may have a viscosity of about 1.0 to about 50.0 cP, about 1.0 to about 40.0 cP, or about 1.0 to about 30.0 cP, for example, about 4 cP, 8 cP, 15 cP, 18 cP, 23 cP, or 26 cP. In example embodiments, the PVOH homopolymers and/or copolymers may have a viscosity of about 1.0 to about 40.0 cP, or about 5 cP to about 23 cP, for example, about 1 cP, 1.5 cP, 2 cP, 2.5 cP, 3 cP, 3.5 cP, 4 cP, 4.5 cP, 5 cP, 5.5 cP, 6 cP, 6.5 cP, 7 cP, 7.5 cP, 8 cP, 8.5 cP, 9 cP, 9.5 cP, 10 cP, 11 cP, 12 cP, 13 cP, 14 cP, 15 cP, 17.5 cP, 18 cP, 19 cP, 20 cP, 21 cP, 22 cP, 23 cP, 24 cP, 25 cP, 26 cP, 27 cP, 28 cP, 29 cP, 30 cP, 31 cP, 32 cP, 33 cP, 34 cP, 35 cP, or 40 cP. In example embodiments, the PVOH homopolymers and/or copolymers may have a viscosity of about 21 cP to 26 cP. In example embodiments, the PVOH homopolymers and/or copolymers can have a viscosity of about 5 cP to about 14 cP. In example embodiments, the PVOH homopolymers and/or copolymers can have a viscosity of about 5 cP to about 23 cP.

The water-soluble polymers, whether polyvinyl alcohol polymers or otherwise, can be blended. When the polymer blend includes a blend of polyvinyl alcohol polymers, the PVOH polymer blend can include a first PVOH polymer (“first PVOH polymer”) which can include a PVOH copolymer or a modified PVOH copolymer including one or more types of anionic monomer units (e.g., a PVOH ter- (or higher co-) polymer) and a second PVOH polymer (“second PVOH polymer”) which can include a PVOH copolymer or a PVOH modified copolymer including one or more types of anionic monomer units (e.g., a PVOH ter- (or higher co-) polymer). In some embodiments, the PVOH polymer blend includes only the first PVOH polymer and the second PVOH polymer (e.g., a binary blend of the two polymers). Alternatively, or additionally, the PVOH polymer blend or a fiber, foam, or film made therefrom can be characterized as being free or substantially free from other polymers (e.g., other water-soluble polymers generally, other PVOH-based polymers specifically, or both). As used herein, “substantially free” means that the first PVOH polymer and the second PVOH polymer make up at least 95 wt. %, at least 97 wt. %, or at least 99 wt. % of the total amount of water-soluble polymers in the water-soluble fiber, foam, or film. In other embodiments, the water-soluble fiber, foam, or film can include one or more additional water-soluble polymers. For example, the PVOH polymer blend can include a third PVOH polymer, a fourth PVOH polymer, a fifth PVOH polymer, etc. (e.g., one or more additional PVOH copolymers or modified PVOH copolymers, with or without anionic monomer units). For example, the water-soluble fiber or film can include at least a third (or fourth, fifth, etc.) water-soluble polymer which is other than a PVOH polymer (e.g., other than PVOH copolymers or modified PVOH copolymers, with or without anionic monomer units). A PVOH homopolymer may also be included in each blend.

### Biodegradability

Polyvinyl alcohol polymers are generally biodegradable as they decompose in the presence of water and enzymes under aerobic, anaerobic, soil, and compost conditions. In general, as the degree of hydrolysis of a polyvinyl alcohol polymer increases up to about 80%, the biodegradation activity of the polyvinyl alcohol polymer increases. Without intending to be bound by theory, it is believed that increasing the degree of hydrolysis above 80% does not appreciably affect biodegradability. Additionally, the stereoregularity of the hydroxyl groups of polyvinyl alcohol polymers has a large effect on the biodegradability activity level and the more isotactic the hydroxyl groups of the polymer sequence, the higher the degradation activity becomes. Without intending to be bound by theory, for soil and/or compost biodegradation, it is believed that a nonwoven substrate or nonwoven web prepared from a polyvinyl alcohol fiber will have higher biodegradation activity levels relative to a water-soluble film prepared from a similar polyvinyl alcohol polymer, due to the increase in the polymer surface area provided by the nonwoven substrate or nonwoven web relative to a film. Further, without intending to be bound by theory, it is believed that while the degree of polymerization of the polyvinyl alcohol polymer has little to no effect on the biodegradability of a film, foam, or nonwoven substrate or web prepared with the polymer, the polymerization temperature may have an effect on the biodegradability of a film, foam substrate, or nonwoven substrate because the polymerization temperature can affect the crystallinity and aggregating status of a polymer. As the crystallinity decreases, the polymer chain hydroxyl groups become less aligned in the polymer structure and the polymer chains become more disordered allowing for chains to accumulate as amorphous aggregates, thereby decreasing availability of ordered polymer structures such that the biodegradation activity is expected to decrease for soil and/or compost biodegradation mechanisms wherein the polymer is not dissolved. Without intending to be bound by theory, it is believed that because the stereoregularity of the hydroxyl groups of polyvinyl alcohol polymers has a large effect on biodegradability activity levels, the substitution of functionalities other than hydroxyl groups (e.g., anionic AMPS functional groups, carboxylate groups, or lactone groups) is expected to decrease the biodegradability activity level, relative to a polyvinyl alcohol copolymer having the same degree of hydrolysis, unless the functional group itself is also biodegradable, in which case biodegradability of the polymer can be increased with substitution. Further, it is believed that while the biodegradability activity level of a substituted polyvinyl alcohol can be less than that of the corresponding homopolymer or copolymer, the substituted polyvinyl alcohol will still exhibit biodegradability.

Methods of determining biodegradation activity are known in the art, for example, as described in Chiellini et al., Progress in Polymer Science, Volume 28, Issue 6, 2003, pp. 963-1014, which is incorporated herein by reference in its entirety. Further methods and standards can be found in ECHA's Annex XV Restriction Report—Microplastics, Version number 1, Jan. 11, 2019, which is incorporated herein by reference in its entirety. Suitable standards include OECD 301B (ready biodegradation), OECD 301B (enhanced biodegradation), OECD 302B (inherent biodegradation), OECD 311(anaerobic), and ASTM D5988 (soil).

In example embodiments, the fibers described herein can be of the standard ready biodegradation or enhanced degradation. As used herein, the term “ready biodegradation” refers to a standard that is met if the material (e.g., a fiber) reached 60% biodegradation (mineralization) within 28 days of the beginning of the test, according to the OECD 301B test as described in the ECHA's Annex XV. As used herein, the term “enhanced biodegradation” refers to a standard that is met if the material (e.g., a fiber) reaches 60% biodegradation within 60 days from the beginning of the test, according to the OECD 301B test as described in the ECHA's Annex XV. In example embodiments, the fibers meet the standards of ready biodegradation.

### Active Cleansing Formulations

In example embodiments, the water-soluble skin cleansing article and, more specifically, the water-soluble core substrate, is configured to contain one or more active cleansing formulations, such as a skin cleansing formulation or a skin wellness formulation, and/or one or more auxiliary agent as described herein. Suitable examples generally include skin cleansing agents, acne treatment medications, emollients, moisturizers, conditioners, wrinkle reducers, unblock (SPF), and rinse aids, alpha hydroxyl acids neutralized in-situ or in formulation to be a specific pH. In example embodiments, the active cleansing formulation is disposed on or coats one or more surfaces of the water-soluble core substrate or is embedded in and/or adhered to the water-soluble core substrate. The water-soluble core substrate may include a single layer, for example, a single layer nonwoven core substrate, or may include a plurality of layers, for example, a sheet of nonwoven core substrate folded in a serpentine arrangement or plied to form layers with the active cleansing formulation disposed between adjacent layers of the water-soluble nonwoven core substrate, for example. As an example, the active cleansing formulation may include, without limitation, one or more of the following: hyaluronic acid, aloe, chamomile extract, lactic acid, citric acid, hydrolyzed collagen, a poly saccharide, a peptide, a surfactant, a foaming agent, a shampoo, a conditioner, a body wash, a face wash, a skin lotion, a skin treatment, a body oil, fragrance, a hair treatment, a bath salt, an essential oil, a bath bomb, an enzyme, a detergent, a surfactant, an emulsifier, a chelant, a pH adjuster, a builder, a structurant, a free fragrance, an encapsulated fragrance, a preservative, a solvent, or a mineral, and/or any ingredient suitable for including in a skin cleansing formulation, a skin wellness formulation, or a personal care formulation.

### Auxiliary Agents

In general, along with the film-forming, foam-forming, and/or fiber-forming material, the fibers, nonwoven substrates or webs, foam substrates, and/or water-soluble films of the disclosure can include auxiliary agents such as, but not limited to, plasticizers, plasticizer compatibilizers, surfactants, lubricants, release agents, fillers, extenders, cross-linking agents, antiblocking agents, antioxidants, detackifying agents, antifoams, nanoparticles such as layered silicate-type nanoclays (e.g., sodium montmorillonite), bleaching agents (e.g., sodium metabisulfite, sodium bisulfite or others), aversive agents such as bitterants (e.g., denatonium salts such as denatonium benzoate, denatonium saccharide, and denatonium chloride; sucrose octaacetate; quinine; flavonoids such as quercetin and naringen; and quassinoids such as quassin and brucine) and pungents (e.g., capsaicin, piperine, allyl isothiocyanate, and resinferatoxin), and other functional ingredients, in amounts suitable for their intended purposes. As used herein and unless specified otherwise, “auxiliary agents” include secondary additives, processing agents, and active agents. Specific such auxiliary agents can be selected from those suitable for use in water-soluble fibers, non-water-soluble fibers, nonwoven webs, foams, or those suitable for use in water-soluble films.

In example embodiments, the fibers, foams, and/or films can be free of auxiliary agents. As used herein and unless specified otherwise, “free of auxiliary agents” with respect to the fiber means that the fiber includes less than about 0.01 wt. %, less than about 0.005 wt. %, or less than about 0.001 wt. % of auxiliary agents, based on the total weight of the fiber. As used herein and unless specified otherwise, “free of auxiliary agents” with respect to the nonwoven substrate or web means that the nonwoven substrate or web includes less than about 0.01 wt. %, less than about 0.005 wt. %, or less than about 0.001 wt. % of auxiliary agents, based on the total weight of the nonwoven substrate or web. In example embodiments, the water-soluble fibers comprise a plasticizer. In example embodiments, the water-soluble fibers comprise a surfactant. In example embodiments, the non-water-soluble fibers comprise a plasticizer. In example embodiments, the non-water-soluble fibers comprise a surfactant. In example embodiments, the nonwoven substrate or web includes a plasticizer. In example embodiments, the nonwoven substrate or web includes a surfactant.

A plasticizer is a liquid, solid, or semi-solid that is added to a material (usually a resin or elastomer) making that material softer, more flexible (by decreasing the glass-transition temperature of the polymer), and easier to process. A polymer can alternatively be internally plasticized by chemically modifying the polymer or monomer. In addition, or in the alternative, a polymer can be externally plasticized by the addition of a suitable plasticizing agent. Water is recognized as a very efficient plasticizer for PVOH and other polymers including, but not limited to, water-soluble polymers; however, the volatility of water makes its utility limited because polymer films need to have at least some resistance (robustness) to a variety of ambient conditions including low and high relative humidity.

The plasticizer can include, without limitation, glycerin, diglycerin, sorbitol, xylitol, maltitol, ethylene glycol, diethylene glycol, triethylene glycol, dipropylene glycol, tetraethylene glycol, propylene glycol, polyethylene glycols up to 1000 MW, neopentyl glycol, trimethylolpropane, polyether polyols, sorbitol, 2-methyl-1,3-propanediol (MPDiol®), ethanolamines, and a mixture thereof.

Surfactants for use in films are well known in the art and can suitably be used in the fibers, foam, films, and/or compositions of the disclosure. Optionally, surfactants are included to aid in the dispersion of the fibers during carding. Optionally, surfactants are included as cleansing aids. Suitable surfactants can include the nonionic, cationic, anionic and zwitterionic classes. Suitable surfactants include, but are not limited to, propylene glycols, diethylene glycols, monoethanolamine, polyoxyethylenated polyoxypropylene glycols, alcohol ethoxylates, alkylphenol ethoxylates, tertiary acetylenic glycols and alkanolamides (nonionics), polyoxyethylenated amines, quaternary ammonium salts and quaternized polyoxyethylenated amines (cationics), alkali metal salts of higher fatty acids containing about 8 to 24 carbon atoms, alkyl sulfates, alkyl polyethoxylate sulfates and alkylbenzene sulfonates (anionics), and amine oxides, N-alkylbetaines and sulfobetaines (zwitterionics). Other suitable surfactants include dioctyl sodium sulfosuccinate, lactylated fatty acid esters of glycerin and propylene glycol, lactylic esters of fatty acids, sodium alkyl sulfates, polysorbate 20, polysorbate 60, polysorbate 65, polysorbate 80, lecithin, acetylated fatty acid esters of glycerin and propylene glycol, and odium lauryl sulfate, acetylated esters of fatty acids, myristyl dimethylamine oxide, trimethyl tallow alkyl ammonium chloride, quaternary ammonium compounds, alkali metal salts of higher fatty acids containing about 8 to 24 carbon atoms, alkyl sulfates, alkyl polyethoxylate sulfates, alkylbenzene sulfonates, monoethanolamine, lauryl alcohol ethoxylate, propylene glycol, diethylene glycol, sodium cocoyl isethionate, sodium lauryl sulfate, glucotain, phoenamids, cola lipid, cocamides, such as cocamide ethanolamines, ethylene oxide based surfactants, saponified oils of avocado and palm, salts thereof and combinations of any of the foregoing. In example embodiments, the surfactant comprises a cocamide. Without intending to be bound by theory, it is believed that a cocamide can aid in foam formation, enhancing the foaming experience of an article comprising a personal care composition. In various embodiments, the amount of surfactant in the fiber is in a range of about 0.01 wt. % to about 10 wt. %, about 0.1 wt. % to about 5 wt. %, about 1.0 wt. % to about 2.5 wt. %, about 0.01 wt. % to about 1.5 wt. %, about 0.1 wt. % to about 1 wt. %, about 0.01 wt. % to 0.25 wt. %, or about 0.10 wt. % to 0.20 wt. %.

In example embodiments, the nonwoven substrates or webs, foam, and/or films of the disclosure can further comprise auxiliary agents such as one or more auxiliary agents in the group of: an exfoliant (chemical exfoliants and mechanical exfoliants), a fragrance and/or perfume microcapsule, an aversive agent, a surfactant, a colorant, an enzyme, a skin conditioner, a de-oiling agent, and a cosmetic agent.

In example embodiments, an auxiliary agent is provided in or on one or more of the nonwoven web, the foam, the plurality of fibers, and the water-soluble film. In example embodiments, an active cleansing formulation is provided on or in one or more of the group of the nonwoven web, the plurality of fibers, and the water-soluble film. In example embodiments, one or more auxiliary agents can be provided on the surface of the nonwoven web. In example embodiments, one or more auxiliary agents can be dispersed among the fibers of the nonwoven web. In example embodiments, one or more auxiliary agents can be dispersed on a face of the nonwoven web. In example embodiments, one or more auxiliary agents can be dispersed in the fibers. In example embodiments, one or more auxiliary agents can be dispersed on the fibers. In example embodiments, one or more auxiliary agents can be provided on a face of the water-soluble film.

The chemical exfoliants, mechanical exfoliants, fragrances and/or perfume microcapsules, aversive agents, surfactants, colorants, proteins, peptides, enzymes, skin conditioners, de-oiling agents, cosmetic agents, or a combination thereof, when present, can be provided in an amount of at least about 1 wt. %, or in a range of about 1 wt. % to about 99 wt. % based on the weight of the polymeric mixture (e.g., fiber forming material or film forming material). In example embodiments, the chemical exfoliants, mechanical exfoliants, fragrances and/or perfume microcapsules, aversive agents, surfactants, colorants, enzymes, skin conditioners, de-oiling agents, and/or cosmetic agents can be provided in an amount sufficient to provide additional functionality to the fiber and/or film, such as exfoliation of human skin. The chemical exfoliants, mechanical exfoliants, fragrances and/or perfume microcapsules, aversive agents, surfactants, colorants, enzymes, skin conditioners, de-oiling agents, cosmetic agents, or a combination thereof, can take any desired form, including as a solid (e.g., powder, granulate, crystal, flake, or ribbon), a liquid, a mull, a paste, a gas, etc., and optionally can be encapsulated, such as microcapsules.

In certain embodiments, the nonwoven substrate or web, foam, and/or film can comprise an enzyme. Suitable enzymes include enzymes categorized in any one of the six conventional Enzyme Commission (EC) categories, i.e., the oxidoreductases of EC 1 (which catalyze oxidation/reduction reactions), the transferases of EC 2 (which transfer a functional group, e.g., a methyl or phosphate group), the hydrolases of EC 3 (which catalyze the hydrolysis of various bonds), the lyases of EC 4 (which cleave various bonds by means other than hydrolysis and oxidation), the isomerases of EC 5 (which catalyze isomerization changes within a molecule) and the ligases of EC 6 (which join two molecules with covalent bonds). Examples of such enzymes include dehydrogenases and oxidases in EC 1, transaminases and kinases in EC 2, lipases, cellulases, amylases, mannanases, and peptidases (a.k.a. proteases or proteolytic enzymes) in EC 3, decarboxylases in EC 4, isomerases and mutases in EC 5 and synthetases and synthases of EC 6. Suitable enzymes from each category are described in, for example, U.S. Pat. No. 9,394,092, the entire disclosure of which is herein incorporated by reference. In certain embodiments, enzymes can include bromeline (pineapple extract), papain (papaya), ficin (fig), actinidin (kiwi), hyaluronidase, lipase, peroxidase, superoxide dismutase, tyrosinase, alkaline phosphatase, or a combination thereof. In example embodiments, the enzyme can be encapsulated in the form of, for example, nanoemulsions, nanocapsules, granules or a combination thereof.

It is contemplated that an enzyme for use herein can come from any suitable source or combination of sources, for example, bacterial, fungal, plant, or animal sources. In one embodiment, a mixture of two or more enzymes will come from at least two different types of sources. For example, a mixture of protease and lipase can come from a bacterial (protease) and fungal (lipase) sources.

Optionally, an enzyme for use herein, including but not limited to any enzyme class or member described herein, is one which works in alkaline pH conditions, e.g., a pH in a range of about 8 to about 11. Optionally, an enzyme for use herein, including but not limited to any enzyme class or member described herein, is one which works in a temperature in a range of about 5° C. to about 45° C.

In example embodiments, the nonwoven substrate or web, foam, and/or film can comprise a protein and/or peptide. Suitable proteins and/or peptides can include, but are not limited to, collagen and/or collagen peptides, or amino acids, for example, aspartic acid, glutamic acid, serine, histidine, glycine, threonine, arginine, alanine, tyrosine, cysteine, valine, methionine, phenylalanine, isoleucine, leucine, lysine, hydroxyproline, or proline.

In example embodiments, the nonwoven substrate or web, foam, and/or film can comprise a colorant. Suitable colorants can include an indicator dye, such as a pH indicator (e.g., thymol blue, bromothymol, thymolphthalein, and thymolphthalein), a moisture/water indicator (e.g., hydrochromic inks or leuco dyes), or a thermochromic ink, wherein the ink changes color when temperature increases and/or decreases. Suitable colorants include, but are not limited to, a triphenylmethane dye, an azo dye, an anthraquinone dye, a perylene dye, an indigoid dye, a food, drug and cosmetic (FD&C) colorant, an organic pigment, an inorganic pigment, or a combination thereof. Examples of colorants include, but are not limited to, FD&C Red #40; Red #3; FD&C Black #3; Black #2; Mica-based pearlescent pigment; FD&C Yellow #6; Green #3; Blue #1; Blue #2; titanium dioxide (food grade); brilliant black; and a combination thereof. Other examples of suitable colorants can be found in U.S. Pat. No. 5,002,789, hereby incorporated by reference in its entirety.

Other embodiments can include one or more fragrances in the nonwoven substrate or webs, foams, and/or films of the disclosure. As used herein, the term “fragrance” refers to any applicable material that is sufficiently volatile to produce a scent. Embodiments including fragrances can include fragrances that are scents pleasurable to humans, or alternatively fragrances that are scents repellant to humans, animals, and/or insects. Suitable fragrances include, but are not limited to, fruits including, but not limited to, lemon, apple, cherry, grape, pear, pineapple, orange, strawberry, raspberry, musk, and flower scents including, but not limited to, lavender-like, rose-like, iris-like and carnation-like. Optionally, the fragrance is one which is not also a flavoring. Other fragrances include herbal scents including, but not limited to, rosemary, thyme, and sage; and woodland scents derived from pine, spruce, and other forest smells. Fragrances may also be derived from various oils, including, but not limited to, essential oils, or from plant materials including, but not limited to, peppermint, spearmint, and the like. Suitable fragrant oils can be found in U.S. Pat. No. 6,458,754, hereby incorporated by reference in its entirety. Suitable fragrant oils include, but are not limited to, 4-(2,2,6-trimethylcyclohex-1-enyl)-2-en-4-one, acetaldehyde phenyletheyl propyl acetal, 2,6,10-trimethyl-9-undecenal, hexanoic acid 2-propenyl ester, 1-octen-3-ol, trans-anethole, iso buthyl (z)-2-methyl-2-butenoate, anisaldehyde diethyl acetal, 3-methyl-5-propyl-cyclohezen-1-one, 2,4-dimethyl-3-cyclohexene-1-carbaldehyde, trans-4-decenal, decanal, 2-pentylcyclopentanone, ethyl anthranilate, eugenol, 3-(3-isopropylphenyl)butanoal, methyl 2-octynoate, isoeugenol, cis-3-hexenyl methyl carbonate, linalool, methyl-2-nonynonate, benzoic acid 2-hydroxymethyl ester, nonal, octanal, 2-nonennitrile, 4-nonanolide, 9-decen-1-ol, and 10-undecen-1-al. Applicable fragrances can also be found in U.S. Pat. Nos. 4,534,981; 5,112,688; 5,145,842; 6,844,302; and Perfumes Cosmetics and Soaps, Second Edition, edited by W. A. Poucher, 1959, all hereby incorporated by reference in their entireties. These fragrances include acacia, cassie, chypre, cyclamen, fern, gardenia, hawthorn, heliotrope, honeysuckle, hyacinth, jasmine, lilac, lily, magnolia, mimosa, narcissus, freshly-cut hay, orange blossom, orchids, reseda, sweet pea, trefle, tuberose, vanilla, violet, wallflower, and the like or any combination thereof.

Fragrances can include perfumes. The perfume may comprise neat perfume, encapsulated perfume, or mixtures thereof. In example embodiments, the perfume includes neat perfume. A portion of the perfume may be encapsulated in a core-shell encapsulate. In other embodiments, the perfume will not be encapsulated in a core/shell encapsulate.

As used herein, the term “perfume” encompasses the perfume raw materials (PRMs) and perfume accords. The term “perfume raw material” as used herein refers to compounds having a molecular weight of at least about 100 g/mol and which are useful in imparting an odor, fragrance, essence or scent, either alone or with other perfume raw materials. As used herein, the terms “perfume ingredient” and “perfume raw material” are interchangeable. The term “accord” as used herein refers to a mixture of two or more PRMs. In example embodiments, any of the perfume accords, perfume raw materials, or fragrances can be encompassed in a microcapsule, termed “perfume microcapsules” as used herein.

Typical PRM comprise inter alia alcohols, ketones, aldehydes, esters, ethers, nitrites, and alkenes, such as terpene. A listing of common PRMs can be found in various reference sources, for example, “Perfume and Flavor Chemicals,” Vols. I and II; Steffen Arctander Allured Pub. Co. (1994) and “Perfumes: Art, Science and Technology,” Miller, P. M. and Lamparsky, D., Blackie Academic and Professional (1994). The PRMs are characterized by their boiling points (B.P.) measured at the normal pressure (760 mm Hg), and their octanol/water partitioning coefficient (P). Based on these characteristics, the PRMS may be categorized as Quadrant I, Quadrant II, Quadrant III, or Quadrant IV perfumes.

In example embodiments, the nonwoven web, foam, and/or film can include an exfoliant. In example embodiments, the exfoliant can comprise a chemical exfoliant or a mechanical exfoliant. Suitable mechanical exfoliants for use herein include, without limitation, apricot shells, sugar, oatmeal, salt, silica, diatomaceous earth, clay, aluminum hydrates, PVOH microbeads, pumice, or a combination thereof. Suitable chemical exfoliants for use herein include, without limitation, alpha hydroxyl acid, beta hydroxyl acid, enzyme, salicylic acid, glycolic acid, citric acid, malic acid, or a combination thereof.

In certain embodiments, the aversive agents, surfactants, colorants, enzymes, skin conditioners, de-oiling agents, cosmetic agents, or a combination thereof, are encapsulated, allowing for controlled release. Suitable microcapsules can include or be made from one or more of melamine formaldehyde, polyurethane, urea formaldehyde, chitosan, polymethyl methacrylate, polystyrene, polysulfone, poly tetrahydrofuran, gelatin, gum arabic, starch, polyvinyl pyrrolidone, carboxymethylcellulose, hydroxyethylcellulose, methylcellulose, arabinogalactan, polyvinyl alcohol, polyacrylic acid, ethylcellulose, polyethylene, polymethacrylate, polyamide, poly (ethylenevinyl acetate), cellulose nitrate, silicones, poly(lactideco-glycolide), paraffin, carnauba, spermaceti, beeswax, stearic acid, stearyl alcohol, glyceryl stearates, shellac, cellulose acetate phthalate, zein, and combinations thereof. In one type of embodiment, the microcapsule is characterized by a mean particle size (e.g., Dv50) of at least about 0.1 micron, or in a range of about 0.1 micron to about 200 microns, for example. In alternate embodiments, the microcapsules can form agglomerates of individual particles, for example wherein the individual particles have a mean particle size of at least about 0.1 micron, or in a range of about 0.1 micron to about 200 microns.

### Water-Soluble Fibers

Water-soluble fibers include fibers and/or fiber forming materials made of any material that, when provided as the sole resin in a film or foam, or sole fiber forming material in a nonwoven, the film, foam, or nonwoven dissolves in 300 seconds or less at temperatures of 80° C. or less, as determined by MSTM-205. The water-soluble fibers can include a single water-soluble polymer or a blend of water-soluble polymers. Suitable water-soluble polymers include, but are not limited to, polyvinyl alcohol homopolymer, polyvinyl alcohol copolymer, modified polyvinyl alcohol copolymer, polyacrylate, water-soluble acrylate copolymer, polyvinyl pyrrolidone, polyethyleneimine, pullulan, water-soluble natural polymer including, but not limited to, guar gum, gum Acacia, xanthan gum, carrageenan, and starch, water-soluble polymer derivatives including, but not limited to, modified starches, ethoxylated starch, and hydroxypropylated starch, copolymers of the forgoing and combinations of any of the foregoing. Yet other water-soluble fibers can include polyalkylene oxides, polyacrylamides, polyacrylic acids and salts thereof, celluloses, cellulose ethers, cellulose esters, cellulose amides, polyvinyl acetates, polycarboxylic acids and salts thereof, polyaminoacids, polyamides, gelatines, methylcelluloses, carboxymethylcelluloses and salts thereof, dextrins, ethylcelluloses, hydroxyethyl celluloses, hydroxypropyl methylcelluloses, maltodextrins, polymethacrylates, and combinations of any of the foregoing. In example embodiments, the water-soluble fibers can include a PVOH copolymer fiber forming material, modified PVOH copolymer fiber forming material, or a combination thereof. In example embodiments, the water-soluble fibers can comprise a sole PVOH copolymer fiber forming material or a blend of PVOH copolymer fiber forming materials. In example embodiments, the water-soluble fibers can comprise a hot water-soluble PVOH copolymer fiber forming material. In further embodiments, the water-soluble fibers can comprise a PVOH copolymer fiber forming material with a viscosity in a range of 5 cP to 23 cP and a degree of hydrolysis in a range of 86% to 92%.

In example embodiments, the water-soluble fibers can include an active cleansing formulations and/or auxiliary agent as described above. In example embodiments, the water-soluble fibers can be substantially free of active cleansing formulations and/or auxiliary agents as described above. In example embodiments, the water-soluble fibers can include a plasticizer as described above. The total amount of the non-water plasticizer provided in the water-soluble fiber can be in a range of about 1 wt. % to about 45 wt. %, or about 5 wt. % to about 45 wt. %, or about 10 wt. % to about 40 wt. %, or about 20 wt. % to about 30 wt. %, about 1 wt. % to about 4 wt. %, or about 1.5 wt. % to about 3.5 wt. %, or about 2.0 wt. % to about 3.0 wt. %, for example about 1 wt. %, about 2.5 wt. %, about 5 wt. %, about 10 wt. %, about 15 wt. %, about 20 wt. %, about 25 wt. %, about 30 wt. %, about 35 wt. %, or about 40 wt. %, based on total fiber weight. In example embodiments, the water-soluble fibers comprise glycerin, sorbitol, or a combination thereof. In example embodiments, the water-soluble fibers comprise glycerin. In example embodiments, the water-soluble fibers comprise sorbitol. In certain embodiments, the water-soluble fibers can include glycerin, for example, in about 10 wt. % based on total fiber weight, and sorbitol, for example, in about 5 wt. % based on the total fiber weight.

In example embodiments, the water-soluble fibers can include a surfactant as described above. In various embodiments, the amount of surfactant in the water-soluble fiber is in a range of about 0.01 wt. %, to about 2.5 wt. %, about 0.1 wt. % to about 2.5 wt. %, about 1.0 wt. % to about 2.0 wt. %, about 0.01 wt. % to 0.25 wt. %, or about 0.10 wt. % to 0.20 wt. %.

In example embodiments, any of the active cleansing formulations and/or auxiliary agents disclosed herein can be added to the fibers of the disclosure. In refinements of the forgoing embodiment, the active cleansing formulations and/or auxiliary agents can be added to the fiber forming material prior to formation of the fiber such that the auxiliary agents are dispersed in the fiber. In addition, and/or in the alternative, active cleansing formulations and/or auxiliary agents can be added to the surface of a fiber after fiber formation (e.g., dispersed on the fibers).

When included in the water-soluble fiber, a colorant can be provided in an amount of 0.01% to 25% by weight of the polymer mixture, such as, 0.02%, 0.05%, 0.1%, 0.5%, 1%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, and 24% by weight of the polymer mixture.

### Non-Water-Soluble Fibers

Non-water-soluble fibers include fibers and/or fiber forming materials made of any material that, when provided in a film as the sole film forming material or provided in a nonwoven web or foam as the sole fiber forming material, the film, the nonwoven web, or the foam does not dissolve in 300 seconds or less at temperatures of 80° C. or less, as determined by MSTM-205. The non-water-soluble fibers can include a sole non-water-soluble polymer fiber forming material or a blend of non-water-soluble polymer fiber forming materials. Suitable non-water-soluble fibers and/or non-water-soluble fiber forming materials include, but are not limited to, cotton, polyester, polyethylene (e.g., high density polyethylene and low density polyethylene), polypropylene, wood pulp, fluff pulp, abaca, viscose, polylactic acid, polyester, nylon 6, insoluble cellulose, insoluble starch, hemp, jute, flax, ramie, sisal, bagasse, banana fiber, lacebark, silk, sinew, catgut, wool, sea silk, mohair, angora, cashmere, collagen, actin, nylon, dacron, rayon, bamboo fiber, modal, diacetate fiber, triacetate fiber, and combinations thereof. In example embodiments, the non-water-soluble fiber forming material and/or non-water-soluble fibers comprise one or more of the group of: cotton, hemp, jute, flax, ramie, sisal, bagasse, banana, lacebark, silk, sinew, catgut, wool, sea silk, mohair, angora, cashmere, collagen, actin, nylon, dacron, rayon, bamboo, modal, diacetate fiber, triacetate fiber, or a combination thereof.

In example embodiments, the non-water-soluble fibers can include an auxiliary agent as described above. In example embodiments, the non-water-soluble fibers can be substantially free of auxiliary agents as described above. In example embodiments, the non-water-soluble fibers can include a plasticizer as described above. The total amount of the non-water plasticizer provided in the non-water-soluble fiber can be in a range of about 1 wt. % to about 45 wt. %, or about 5 wt. % to about 45 wt. %, or about 10 wt. % to about 40 wt. %, or about 20 wt. % to about 30 wt. %, about 1 wt. % to about 4 wt. %, or about 1.5 wt. % to about 3.5 wt. %, or about 2.0 wt. % to about 3.0 wt. %, for example, about 1 wt. %, about 2.5 wt. %, about 5 wt. %, about 10 wt. %, about 15 wt. %, about 20 wt. %, about 25 wt. %, about 30 wt. %, about 35 wt. %, or about 40 wt. %, based on total fiber weight. In example embodiments, the non-water-soluble fibers comprise glycerin, sorbitol, or a combination thereof. In example embodiments, the non-water-soluble fibers comprise glycerin. In example embodiments, the non-water-soluble fibers comprise sorbitol. In certain embodiments, the non-water-soluble fibers can include a plasticizer such as glycerin, for example in about 10 wt % based on total fiber weight, and sorbitol, for example in about 5 wt % based on the total fiber weight.

In example embodiments, the non-water-soluble fibers can include a surfactant as described above. In various embodiments, the amount of surfactant in the water-soluble fiber is in a range of about 0.01 wt. %, to about 2.5 wt. %, about 0.1 wt. % to about 2.5 wt. %, about 1.0 wt. % to about 2.0 wt. %, about 0.01 wt. % to 0.25 wt. %, or about 0.10 wt. % to 0.20 wt. %.

In example embodiments, any of the auxiliary agents disclosed herein can be added to the fibers of the disclosure. In refinements of the forgoing embodiment can be added to the fiber forming material prior to formation of the fiber such that the auxiliary agents can be added to the surface of a fiber after fiber formation. In refinements of the foregoing embodiments, the auxiliary agents can be added to a surface of the fiber after fiber formation.

When included in the non-water-soluble fiber, the colorant can be provided in an amount of 0.01% to 25% by weight of the polymer mixture, such as, 0.02%, 0.05%, 0.1%, 0.5%, 1%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, and 24% by weight of the polymer mixture.

### Nonwoven Webs or Nonwoven Substrates

The nonwoven web or nonwoven substrates of the disclosure can be water-soluble, non-water-soluble, or at least partially non-water-soluble. The single unit dose article of the disclosure can include a nonwoven web, wherein at least a portion of the nonwoven web is soluble in water at a temperature in a range of about 0° C. to about 20° C. according to MSTM-205, or at least a portion of the nonwoven web is not soluble in water at a temperature of 20° C. or less according to MSTM-205, or the nonwoven web is not soluble in water at a temperature of 20° C. or less according to MSTM-205, or the nonwoven web is soluble in water at a temperature in a range of about 0° C. to about 20° C. according to MSTM-205. It will be understood that “at least a portion” of a nonwoven web is soluble (or not soluble) at a given temperature if the nonwoven web includes in the plurality of the fibers, a fiber type which when provided in a nonwoven as the sole fiber type, the nonwoven web consisting of that fiber type is soluble (or not soluble) at the given temperature, according to MSTM-205.

The nonwoven web of the disclosure includes a plurality of fibers. A nonwoven web refers to an arrangement of fibers bonded to one another, wherein the fibers are neither woven nor knitted. The plurality of fibers can be arranged in any orientation. In example embodiments, the plurality of fibers are arranged randomly (i.e., do not have an orientation). In example embodiments, the plurality of fibers are arranged in a unidirectional orientation. In example embodiments, the plurality of fibers are arranged in a bidirectional orientation. In some embodiments, the plurality of fibers are multi-directional, having different arrangements in different areas of the nonwoven web.

The plurality of fibers in any given nonwoven web can include any fiber-forming materials disclosed herein. The nonwoven web can include (1) a single fiber type including a single fiber forming material, (2) a single fiber type including a blend of fiber forming materials, (3) a blend of fiber types, each fiber type including a single fiber forming material, (4) a blend of fiber types, each fiber type including a blend of fiber forming materials, or (5) a blend of fiber types, each fiber type including a single fiber forming material or a blend of fiber forming materials. In example embodiments including a blend of fiber types, the different fiber types can have a difference in one or more of the group of length to diameter ratio (L/D), tenacity, shape, rigidness, elasticity, solubility, melting point, glass transition temperature (T_{g}), fiber forming material chemistries, and color. In certain embodiments, the plurality of fibers can comprise two or more types of water-soluble fibers. In example embodiments, the plurality of fibers can comprise at least one fiber type comprising at least one type of water-soluble fiber forming materials and at least one fiber type comprising at least type of one non-water-soluble fiber. In example embodiments, the plurality of fibers can comprise two or more fiber types comprising at least one type of non-water-soluble fiber forming material.

In example embodiments, the nonwoven web can further comprise any active cleansing formulations and/or auxiliary agents as disclosed herein for fibers and/or films. In example embodiments, the active cleansing formulations and/or auxiliary agents can be added to the fiber itself, to the nonwoven web during carding of the nonwoven web, to the nonwoven web prior to bonding (e.g., after carding), to the nonwoven web after bonding, or a combination thereof. The active cleansing formulations and/or auxiliary agents added to the fibers during carding can be distributed throughout the nonwoven web. The active cleansing formulations and/or auxiliary agents added to the nonwoven web after carding but prior to bonding can be selectively added to one or both faces of the nonwoven web.

The active cleansing formulations and/or auxiliary agents can be applied to one or more faces or surfaces of a nonwoven web or to an article containing same, e.g., a packet, by any suitable means. In example embodiments, the active cleansing formulations and/or auxiliary agents are in powder form. In refinements of the foregoing embodiment, one or more stationary powder spray guns are used to direct the powder stream towards the web, from one or more than one direction, while the web is transported through the coating zone by means of a belt conveyor. In example embodiments, a web or packet is conveyed through a suspension of the powder in air. In example embodiments, the webs are tumble-mixed with the powder in a trough-like apparatus. In example embodiments, which can be combined with any other embodiment, electrostatic forces are employed to enhance the attraction between the powder and the web. This type of process may be based on negatively charging the powder particles and directing these charged particles to the grounded webs. In other alternative embodiments, the powder is applied to the web by a secondary transferring tool including, but not limited to, rotating brushes, which are in contact with the powder or by powdered gloves, which can transfer the powder from a container to the web. In yet another embodiment, the powder is applied by dissolving or suspending the powder in a non-aqueous solvent or carrier, which is then atomized and sprayed onto the web. In one embodiment, the solvent or carrier subsequently evaporates, leaving the active agent powder behind. In certain embodiments, the powder is applied to the web in an accurate dose. These embodiments utilize closed-system dry lubricant application machinery, such as PekuTECH's powder applicator PM 700 D. In this process, the powder, optionally batch-wise or continuously, is fed to a feed trough of application machinery. The webs are transferred from the output belt of a standard rotary drum pouch machine onto a conveyor belt of the powder application machine, wherein a controlled dosage of the powder is applied to the web. The web can thereafter be conveyed to a suitable packaging process.

In example embodiments wherein the auxiliary agents are in liquid form or in a solution, the foregoing can be dispersed among the fibers, dispersed on a face or surface of the nonwoven web, or a combination thereof, for example, by spin casting, spraying a solution such as an aerosolized solution, roll coating, flow coating, curtain coating, extrusion, knife coating, and combinations thereof.

In example embodiments, the active cleansing formulations and/or auxiliary agents, such as chemical exfoliants, mechanical exfoliants, fragrances and/or perfume microcapsules, aversive agents, surfactants, colorants, enzymes, skin conditioners, de-oiling agents, cosmetic agents, or a combination thereof, when present in the nonwoven web, are in an amount of at least about 1 wt. %, or in a range of about 1 wt. % to about 99 wt. %, provides additional functionality to the nonwoven web. The chemical exfoliants, mechanical exfoliants, fragrances and/or perfume microcapsules, aversive agents, surfactants, colorants, enzymes, skin conditioners, de-oiling agents, cosmetic agents, or a combination thereof, can take any desired form, including as a solid (e.g., powder, granulate, crystal, flake, or ribbon), a liquid, a mull, a paste, a gas, etc., and optionally can be encapsulated.

In example embodiments, the nonwoven web can be colored, pigmented, and/or dyed to provide an improved aesthetic effect relative to water-soluble films. Suitable colorants for use in the nonwoven web can include an indicator dye, such as a pH indicator (e.g., thymol blue, bromothymol, thymolphthalein, and thymolphthalein), a moisture/water indicator (e.g., hydrochromic inks or leuco dyes), or a thermochromic ink, wherein the ink changes color when temperature increases and/or decreases. Suitable colorants include, but are not limited to, a triphenylmethane dye, an azo dye, an anthraquinone dye, a perylene dye, an indigoid dye, a food, drug and cosmetic (FD&C) colorant, an organic pigment, an inorganic pigment, or a combination thereof. Examples of colorants include, but are not limited to, FD&C Red #40; Red #3; FD&C Black #3; Black #2; Mica-based pearlescent pigment; FD&C Yellow #6; Green #3; Blue #1; Blue #2; titanium dioxide (food grade); brilliant black; and a combination thereof.

In example embodiments, the nonwoven web can include any of the surfactants disclosed herein. In example embodiments, the nonwoven web can comprise one or more of the group of: sodium cocoyl isethionate, glucotain, phoenamids, cola lipid, cocamides, such as cocamide ethanolamines, ethylene oxide-based surfactants, and saponified oils of avocado and palm.

The nonwoven webs of the disclosure can have any thickness. Suitable thicknesses can include, but are not limited to, about 5 microns (μm) to about 10,000 μm (1 cm), about 5 μm to about 5,000 μm, about 5 μm to about 1,000 μm, about 5 μm to about 500 μm, about 200 μm to about 500 μm, about 5 μm to about 200 μm, about 20 μm to about 100 μm, or about 40 μm to about 90 μm, or about 50 μm to 80 μm, or about or about 60 μm to 65 μm, for example, 50 μm, 65 μm, 76 μm, or 88 μm. The nonwoven webs of the disclosure can be characterized as high loft or low loft. Loft refers to the ratio of thickness to mass per unit area (i.e., basis weight). High loft nonwoven webs can be characterized by a high ratio of thickness to mass per unit area. As used herein, “high loft” refers to a nonwoven web of the disclosure having a basis weight as defined herein and a thickness exceeding 200 μm. The thickness of the nonwoven web can be determined according to ASTM D5729-97, ASTM D5736, and/or ISO 9073-2:1995 and can include, for example, subjecting the nonwoven web to a load of 2 N and measuring the thickness. High loft materials can be used according to known methods in the art, for example, cross-lapping, which uses a cross-lapper to fold the unbonded web over onto itself to build loft and basis weight. Without intending to be bound by theory, in contrast to water-soluble films wherein the solubility of the film can be dependent on the thickness of the film, the solubility of a nonwoven web is not believed to be dependent on the thickness of the web. In this regard, it is believed that because the individual fibers provide a higher surface area than a water-soluble film, regardless of the thickness of the nonwoven web, the parameter that limits approach of water to the fibers and, thereby, dissolution of the fibers is the basis weight (i.e., fiber density in the nonwoven).

In general, the coefficient of dynamic friction and the ratio of the coefficient of static friction to the coefficient of dynamic friction for a nonwoven web of the disclosure will be lower than the coefficient of dynamic friction and the ratio of the coefficient of static friction to the coefficient of dynamic friction for a water-soluble film due to the increased surface roughness of the nonwoven web relative to a water-soluble film, which provides decreased surface contact to the nonwoven web. Advantageously, this surface roughness can provide an improved feel to the consumer (i.e., a cloth-like hand-feel instead of a rubbery hand-feel), improved aesthetics (i.e., less glossy than a water-soluble film), and/or facilitate processability which may require drawing the web along a surface of the processing equipment/mold. Accordingly, in example embodiments, the water-soluble fibers and/or non-water-soluble fibers are sufficiently coarse to provide a surface roughness to the resulting nonwoven web without being so coarse as to produce drag.

The solubility in water of the nonwoven webs of the disclosure is a function of the type of fiber(s) used to prepare the web as well as the basis weight of the web. Without intending to be bound by theory, it is believed that the solubility profile of a nonwoven web follows the same solubility profile of the fiber(s) used to prepare the nonwoven web, and the solubility profile of the fiber generally follows the same solubility profile of the polymer(s) from which the fiber is prepared. For example, for nonwoven webs comprising PVOH fibers, the degree of hydrolysis of the PVOH polymer can be chosen such that the water-solubility of the nonwoven web is also influenced. At a given temperature, as the degree of hydrolysis of the PVOH polymer increases from partially hydrolyzed (88% DH) to fully hydrolyzed (≥98% DH), water solubility of the polymer generally decreases. Thus, in example embodiments, the nonwoven web can be cold water-soluble. For a co-poly(vinyl acetate vinyl alcohol) polymer that does not include any other monomers (e.g., not copolymerized with an anionic monomer) a cold water-soluble web, soluble in water at a temperature of less than 10° C., can include fibers of PVOH with a degree of hydrolysis in a range of about 75% to about 90%, or in a range of about 75% to about 89%, or in a range of about 80% to about 90%, or in a range of about 85% to about 90%, or in a range of about 90% to about 99.5%. In other example embodiments, the nonwoven web can be hot water-soluble. For example, a co-poly(vinyl acetate vinyl alcohol) polymer that does not include any other monomers (e.g., not copolymerized with an anionic monomer), a hot water-soluble web can be soluble in water at a temperature of at least about 60° C., by including fibers of PVOH with a degree of hydrolysis of at least about 98%.

Modification of a PVOH polymer generally increases the solubility of the PVOH polymer. Thus, it is expected that at a given temperature the solubility of a nonwoven web or film prepared from a modified PVOH polymer would be higher than that of a nonwoven web or film prepared from a PVOH copolymer having the same degree of hydrolysis as the modified PVOH copolymer. Following these trends, a nonwoven web having specific solubility characteristics can be designed by blending polymers within the fibers and/or blending fibers within the nonwoven web. Further, as described herein, the nonwoven web includes a plurality of fibers that may, in some cases, include two or more fiber types that differ in solubility.

Inclusion of non-water-soluble fiber and/or non-water-soluble fiber forming material in the plurality of fibers of a nonwoven web can also be used to design a nonwoven web having specific solubility and/or prolonged release properties. Without intending to be bound by theory, it is believed that as the weight percent of non-water-soluble fiber included in a nonwoven web is increased (based on the total weight of the nonwoven web), the solubility of the nonwoven web generally decreases and the prolonged release properties of a pouch comprising a nonwoven web generally increases. Upon contact with water at a temperature at or above the solubility temperature of the water-soluble fiber, a nonwoven web comprising water-soluble fiber and non-water-soluble fiber will begin to disperse as the water-soluble fiber dissolves, thereby breaking down the web structure and/or increasing the pore size of the pores of the nonwoven web. The larger the break-down of the web structure or increase in the pore size, the faster the active cleansing formulation will be released. Similarly, prolonged release of the active cleansing formulation in the nonwoven web of the disclosure can be achieved by using a blend of water-soluble fibers having different solubility properties and/or different solubility temperatures. Once the faster dissolving fiber has dissolved, thereby breaking up the web, the less soluble fibers will have a larger surface area exposed, facilitating dissolution of the less soluble fibers and release of the active cleansing formulation. In example embodiments wherein the nonwoven web includes water-soluble fibers and non-water-soluble fibers, the ratio of soluble fibers to non-water-soluble fibers is not particularly limited. The water-soluble fibers can comprise about 1% to about 99%, about 20% to about 80%, about 40% to about 90%, about 50% to about 90%, or about 60% to about 90% by weight, of the total weight of the plurality of fibers, and the non-water-soluble fibers can comprise about 1% to about 99%, about 20% to about 80%, about 10% to about 60%, about 10% to about 50%, or about 10% to about 40% by weight, of the total weight of the fibers. In example embodiments, the plurality of fibers comprise about 10% to about 80% water-soluble fibers by weight, based on the total weight of the fibers and the balance being non-water-soluble fibers.

In example embodiments, the nonwoven web, the plurality of fibers, the foam, the water-soluble film, or a combination thereof, disclosed herein can comprises a biodegradable polymer. In certain embodiments, the plurality of fibers can comprise non-water-soluble fiber forming materials that are biodegradable. In example embodiments, the plurality of fibers can comprise first fibers that are non-water-soluble biodegradable fibers, and second fibers that are soluble in water at a temperature of about 10° C. to about 20° C. according to MSTM-205 or not soluble in water at a temperature of about 30° C. or less according to MSTM-205, according to MSTM-205. In example embodiments, the nonwoven web is non-water-soluble and biodegradable.

In example embodiments, the nonwoven web is biodegradable. As used herein, when the nonwoven web is said to be biodegradable, at least 50% of the nonwoven web is biodegradable, for example, at least 60%, at least 70%, at least 80%, at least 90%, or 100%, of the nonwoven web is biodegradable.

The nonwoven web as disclosed herein can comprise the plurality of fibers comprising a first fiber type and a second fiber type, wherein the first and second fiber types have a difference in diameter, length, tenacity, shape, rigidness, elasticity, solubility, melting point, glass transition temperature (T_{g}), chemical composition, color, or a combination thereof. In example embodiments, the first fiber type can comprise a PVOH homopolymer fiber forming material, a PVOH copolymer fiber forming material, a modified PVOH copolymer fiber forming material, or a combination thereof. In example embodiments, the first fiber type can comprise two or more PVOH homopolymer fiber forming materials, two or more PVOH copolymer fiber forming materials, a PVOH copolymer fiber forming material, or a combination thereof. In example embodiments, the second fiber type can comprise a PVOH homopolymer fiber forming material, a PVOH copolymer fiber forming material, a PVOH copolymer fiber forming material, or a combination thereof. In example embodiments, the second fiber type can comprise two or more PVOH homopolymer fiber forming materials, two or more PVOH copolymer fiber forming materials, two or more modified PVOH copolymer fiber forming material, or a combination thereof. In example embodiments, the first fiber type and/or the second fiber type are non-water-soluble fiber forming material. In example embodiments, the first fiber type can comprise a non-water-soluble polymer fiber forming material and the second fiber type can comprise a polyvinyl alcohol fiber forming material that, when provided as the sole fiber forming material of a nonwoven web or as a film, the resulting web or film is soluble in water at a temperature in a range of about 0° C. to about 20° C. according to MSTM-205. In example embodiments, the first fiber type can comprise a non-water-soluble polymer fiber forming material and the second fiber type can comprise a PVOH homopolymer or copolymer fiber forming material that, when provided as the sole fiber forming material of a nonwoven web or as a film, the resulting web or film is not soluble in water at a temperature of 20° C. or less according to MSTM-205. In example embodiments, the first fiber type comprises two or more polyvinyl alcohol copolymer fiber forming materials, two or more modified polyvinyl alcohol copolymer fiber forming materials, or a combination of polyvinyl alcohol copolymer fiber forming materials and modified polyvinyl alcohol copolymer fiber forming materials. In example embodiments, the second fiber type comprises two or more polyvinyl alcohol copolymer fiber forming materials, two or more modified polyvinyl alcohol copolymer fiber forming materials, or a combination of polyvinyl alcohol copolymer fiber forming materials and modified polyvinyl alcohol copolymer fiber forming materials.

The plurality of fibers comprised in the nonwoven webs of the disclosure can have any tenacity. The tenacity of the fiber correlates to the coarseness of the fiber. As the tenacity of the fiber decreases, the coarseness of the fiber increases. Fibers used to prepare the nonwoven webs of the disclosure can have a tenacity in a range of about 1 to about 100 cN/dtex, or about 1 to about 75 cN/dtex, or about 1 to about 50 cN/dtex, or about 1 to about 45 cN/dtex, or about 1 to about 40 cN/dtex, or about 1 to about 35 cN/dtex, or about 1 to about 30 cN/dtex, or about 1 to about 25 cN/dtex, or about 1 to about 20 cN/dtex, or about 1 to about 15 cN/dtex, or about 1 to about 10 cN/dtex, or about 3 to about 8 cN/dtex, or about 4 to about 8 cN/dtex, or about 6 to about 8 cN/dtex, or about 4 to about 7 cN/dtex, or about 10 to about 20, or about 10 to about 18, or about 10 to about 16, or about 1 cN/dtex, about 2 cN/dtex, about 3 cN/dtex, about 4 cN/dtex, about 5 cN/dtex, about 6 cN/dtex, about 7 cN/dtex, about 8 cN/dtex, about 9 cN/dtex, about 10 cN/dtex, about 11 cN/dtex, about 12 cN/dtex, about 13 cN/dtex, about 14 cN/dtex, or about 15 cN/dtex. In example embodiments, the plurality of fibers can have a tenacity in a range of about 3 cN/dtex to about 15 cN/dtex, or about 5 cN/dtex to about 12 cN/dtex, or about 5 cN/dtex to about 10 cN/dtex.

The tenacity of the nonwoven web can be the same or different from the tenacity of the plurality of fibers used to prepare the web. Without intending to be bound by theory, it is believed that the tenacity of the nonwoven web is related to the strength of the nonwoven web, wherein a higher tenacity provides a higher strength to the nonwoven web. The tenacity of the nonwoven web can be modified by using fibers having different tenacities. The tenacity of the nonwoven web may also be affected by processing. The nonwoven webs of the disclosure have relatively high tenacities, i.e., the nonwoven web is a self-supporting web that can be used as the sole material for preparing an article and/or pouch. In contrast, nonwoven webs prepared according to melt blown, electro-spinning, and/or rotary spinning processes may have low tenacities and may not be self-supporting or capable of being used as a sole web for forming an article or pouch.

The fibers used to prepare the nonwoven webs of the disclosure can have any fineness. The fineness of the fiber correlates to how many fibers are present in a cross-section of a yarn of a given thickness. The fineness of a fiber can be measured by the linear mass density, a measure of the ratio of fiber mass per unit length. The main physical unit of linear mass density is 1 tex, which is equal to 1000 m of fiber weighing 1 g. The unit dtex is used, representing 1 g/10,000 m of fiber. The linear mass density can be selected to provide a nonwoven web having suitable stiffness/hand-feel of the nonwoven web, torsional rigidity, reflection and interaction with light, absorption of dye and/or other actives/additives, ease of fiber spinning in the manufacturing process, and uniformity of the finished article. As the linear mass density of the fibers increases, the nonwovens resulting therefrom demonstrate higher uniformity, improved tensile strengths, extensibility, and luster. Additionally, without intending to be bound by theory it is believed that finer fibers will lead to slower dissolution times as compared to larger fibers based on density. Further, without intending to be bound by theory, when a blend of fiber types is used, the average linear mass density can be determined using a weighted average of the individual fiber types. Fibers can be characterized as very fine (dtex≤1.22), fine (1.22≤dtex≤1.54), medium (1.54≤dtex≤1.93), slightly coarse (1.93≤dtex≤2.32), and coarse (dtex≥2.32). The nonwoven web of the disclosure can include fibers that are very fine, fine, medium, slightly coarse, or a combination thereof. In example embodiments, the nonwoven web has an average linear mass density in a range of about 1 dtex to about 5 dtex, or about 1 dtex to about 3 dtex, or about 1.5 dtex to about 2.5 dtex. In example embodiments, the nonwoven web comprises a blend of fibers wherein first fiber comprises 1.7 dtex average linear mass density and second fiber comprises 2.2 dtex average linear mass density.

The plurality of fibers used to prepare the nonwoven webs of the disclosure have a diameter in a range of about 10 microns to 300 microns, for example, at least 10 microns, at least 25 microns, at least 50 microns, at least 100 microns, or at least 125 microns and up to about 300 microns, up to about 275 microns, up to about 250 microns, up to about 225 microns, or up to about 200 microns. In example embodiments, the plurality of fibers used to prepare the nonwoven webs of the disclosure can have a diameter greater than 100 microns to about 300 microns. In example embodiments, the diameters of the plurality of fibers used to prepare the nonwoven webs of the disclosure have diameters that are substantially uniform. In example embodiments, the one or more fiber types can have a mean diameter in a range of about 10 microns to about 300 microns, or about 50 microns to 200 microns, or about 50 microns to about 100 microns.

The plurality of fibers used to prepare the nonwoven webs of the disclosure can be of any length. In example embodiments, the length of the plurality of fibers can be in a range of about 30 millimeters (mm) to about 100 mm, about 10 mm to about 60 mm, or about 30 mm to about 60 mm, for example, at least about 30 mm, at least about 35 mm, at least about 40 mm, at least about 45 mm, or at least about 50 mm, and up to about 100 mm, up to about 95 mm, up to about 90 mm, up to about 80 mm, up to about 70 mm, or up to about 60 mm. In example embodiments, the length of the plurality of fibers can be less than about 30 mm or in a range of about 0.25 mm to less than about 30 mm, for example, at least about 0.25 mm, at least about 0.5 mm, at least about 0.75 mm, at least about 1 mm, at least about 2.5 mm, at least about 5 mm, at least about 7.5 mm, or at least about 10 mm and up to about 29 mm, up to about 28 mm, up to about 27 mm, up to about 26 mm, up to about 25 mm, up to about 20 mm, or up to about 15 mm. In example embodiments, the fibers have an average length of about 30 mm to about 100 mm, or about 30 mm to about 60 mm. In example embodiments, the nonwoven web comprises a blend of fiber types wherein first fiber type comprises a length of about 38 mm and second fiber type comprises a length of about 54 mm.

The plurality of fibers used to prepare the nonwoven webs of the disclosure can have any length to diameter (L/D) ratio. Advantageously, the tactility of a nonwoven web of the disclosure can be controlled using the L/D ratio of the fibers and the respective amounts of fibers having various L/D ratios in the nonwoven composition. As the L/D of the fiber decreases, the stiffness and resistance to bending increases, providing a rougher hand feel. The fibers of the disclosure impart a rough feel to a nonwoven web including same, when the fibers have a low L/D ratio in a range of about 0.5 to about 15, or about 0.5 to about 25, or about 1 to about 5. Such low L/D fibers can be provided in a nonwoven web in an amount in a range of about 0 to about 50% by weight, based on the total weight of the fibers in the nonwoven web, for example, in a range of about 0.5 wt. % to about 25 wt. %, or about 1 wt. % to about 15 wt. %. If the amount of low L/D fibers in a nonwoven web is not known, the amount can be estimated by visual inspection of a micrograph of a nonwoven web. In example embodiments wherein a first fiber includes a blend of fiber forming materials including a first polyvinyl alcohol fiber forming material, at least a portion of the first fibers can have a L/D ratio of about 0.5 to about 25, or about 0.5 to about 15, or about 1 to about 5.

Pore sizes can be determined using high magnification and ordered surface analysis techniques including, but not limited to Brunauer-Emmett-Teller theory (BET), small angle X-ray scattering (SAXS), and molecular adsorption.

Nonwoven webs can be characterized by basis weight. The basis weight of a nonwoven web is the mass per unit area of the nonwoven web. Basis weight can be modified by varying manufacturing conditions, as is known in the art. A nonwoven web can have the same basis weight prior to and after bonding. Alternatively, the bonding method can change the basis weight of the nonwoven web. For example, wherein bonding occurs through the application of heat and pressure, the thickness of the nonwoven (and, thus, the area of the nonwoven) can be decreased, thereby increasing the basis weight. Accordingly, as used herein and unless specified otherwise, the basis weight of a nonwoven refers to the basis weight of the nonwoven after bonding.

The nonwoven webs of the disclosure can have any basis weight in a range of about 0.1 g/m² to about 700 g/m², about 0.5 g/m² to about 600 g/m², about 1 g/m² to about 500 g/m², about 1 g/m² to about 400 g/m², about 1 g/m² to about 300 g/m², about 1 g/m² to about 200 g/m², about 1 g/m² to about 100 g/m², about 30 g/m² to about 100 g/m², about 20 g/m² to about 100 g/m², about 20 g/m² to about 80 g/m², or about 25 g/m² to about 70 g/m².

Further, as the basis weight of the web increases, the rate of dissolution of the web decreases, provided the fiber composition and web thickness remain constant, as there is more material to be dissolved. For example, at a given temperature, a water-soluble web prepared from fibers comprising PVOH polymer(s) and having a basis weight of, e.g., 40 g/m², is expected to dissolve slower than an otherwise-identical water-soluble web having a basis weight of, e.g., 30 g/m². Accordingly, basis weight can also be used to modify the solubility characteristics of the nonwoven web. The nonwoven web can have any basis weight in a range of about 1 g/m² to about 700 g/m², about 1 g/m² to about 600 g/m², about 1 g/m² to about 500 g/m², about 1 g/m² to about 400 g/m², about 1 g/m² to about 300 g/m², about 1 g/m² to about 200 g/m², about 10 g/m² to about 100 g/m², about 30 g/m² to about 100 g/m², about 20 g/m² to about 100 g/m², about 20 g/m² to about 80 g/m², about 25 g/m² to about 70 g/m², or about 40 g/m² to about 60 g/m².

The nonwoven web of the disclosure can be used as a single layer or can be layered with other nonwoven webs or can be in the form of a laminate with a water-soluble film. In some embodiments, the nonwoven web includes a single layer of nonwoven web. In some embodiments, the nonwoven web is a multilayer nonwoven web comprising two or more layers of nonwoven webs. The two or more layers can be laminated to each other. In refinements of the foregoing embodiment, the two or more layers can be the same (e.g., be prepared from the same fibers and basis weight). In refinements of the foregoing embodiment, the two or more layers can be different (e.g., be prepared from different types of fibers, fiber chemistries, and/or have different basis weights).

A multilayer nonwoven web can have a basis weight that is the sum of the basis weights of the individual layers. Accordingly, a multilayer nonwoven web will take longer to dissolve than any of the individual layers provided as a single layer.

### Water-Soluble Foams

In example embodiments, a suitable water-soluble foam includes any suitable resin chemistry, such as a copolymer, maleic anhydride (MA) modified PVOH polymer, monomethyl maleate (MMM) Modified PVOH polymer, 2-methylacrylamido-2-methylpropanesulfonic acid (AMPS) Modified PVOH, cellulose and cellulose derivatives, polyvinylpyrrolidone (PVP), proteins, casein, soy, or any water-dispersible or water-soluble resin. In certain embodiments, the water-soluble foam substrate has a thickness of 3 microns to 3000 microns and can be formed using any suitable manufacturing process known in the foam manufacturing art including, without limitation, a cast, extruded, melt processed, coated, chemically blown, mechanically aerated, air injected, turbulent extrusion process. The water-soluble foam substrate may be porous or non-porous and cold water-soluble or hot water-soluble. The construction of the water-soluble foam substrate may include, for example, folded layers or plies, stacked layers or plies, or rolled layers or plies.

In example embodiments, the water-soluble foam substrate can further comprise any auxiliary agents as disclosed herein for nonwoven webs, fibers and/or films. The auxiliary agents can be applied to one or more faces of a water-soluble foam substrate or to an article containing same, e.g., a packet, by any suitable means. In example embodiments, the auxiliary agents are in powder form. In refinements of the foregoing embodiment, one or more stationary powder spray guns are used to direct the powder stream towards the water-soluble foam substrate or a packet, from one or more than one direction, while the water-soluble foam substrate or packet is transported through the coating zone by means of a belt conveyor. In example embodiments, a water-soluble foam substrate or packet is conveyed through a suspension of the powder in air. In example embodiments, the water-soluble foam substrate or packets are tumble-mixed with the powder in a trough-like apparatus. In example embodiments, which can be combined with any other embodiment, electrostatic forces are employed to enhance the attraction between the powder and the packet or water-soluble foam substrate. This type of process may be based on negatively charging the powder particles and directing these charged particles to the grounded packets or water-soluble foam substrates. In other alternative embodiments, the powder is applied to the water-soluble foam substrate or packet by a secondary transferring tool including, but not limited to, rotating brushes which are in contact with the powder or by powdered gloves which can transfer the powder from a container to the water-soluble foam substrate or the packet. In yet another embodiment, the powder is applied by dissolving or suspending the powder in a non-aqueous solvent or carrier which is then atomized and sprayed onto the water-soluble foam substrate or packet. In one embodiment, the solvent or carrier subsequently evaporates, leaving the active agent powder behind. In certain embodiments, the powder is applied to the water-soluble foam substrate or packet in an accurate dose. These embodiments utilize closed-system dry lubricant application machinery, such as PekuTECH's powder applicator PM 700 D. In this process, the powder, optionally batch-wise or continuously, is fed to a feed trough of application machinery. The water-soluble foam substrates or packets are transferred from the output belt of a standard rotary drum pouch machine onto a conveyor belt of the powder application machine, wherein a controlled dosage of the powder is applied to the water-soluble foam substrate or packet. The water-soluble foam substrate or packet can thereafter be conveyed to a suitable secondary packaging process.

In example embodiments wherein the auxiliary agents are in liquid form or in a solution, the foregoing can be dispersed in the water-soluble foam substrate, dispersed on a face of the water-soluble foam substrate, or a combination thereof, for example, by spin casting, spraying a solution such as an aerosolized solution, roll coating, flow coating, curtain coating, extrusion, knife coating, and combinations thereof.

The auxiliary agents, such as chemical exfoliants, mechanical exfoliants, fragrances and/or perfume microcapsules, aversive agents, surfactants, colorants, enzymes, skin conditioners, de-oiling agents, cosmetic agents, or a combination thereof, when present in the water-soluble foam substrate, are in an amount of at least about 1 wt. %, or in a range of about 1 wt. % to about 99 wt. %, provides additional functionality to the water-soluble foam substrate. The chemical exfoliants, mechanical exfoliants, fragrances and/or perfume microcapsules, aversive agents, surfactants, colorants, enzymes, skin conditioners, de-oiling agents, cosmetic agents, or a combination thereof, can take any desired form, including as a solid (e.g., powder, granulate, crystal, flake, or ribbon), a liquid, a mull, a paste, a gas, etc., and optionally can be encapsulated.

In example embodiments, the water-soluble foam substrate can be colored, pigmented, and/or dyed to provide an improved aesthetic effect relative to water-soluble films. Suitable colorants for use in the water-soluble foam substrate can include an indicator dye, such as a pH indicator (e.g., thymol blue, bromothymol, thymolphthalein, and thymolphthalein), a moisture/water indicator (e.g., hydrochromic inks or leuco dyes), or a thermochromic ink, wherein the ink changes color when temperature increases and/or decreases. Suitable colorants include, but are not limited to, a triphenylmethane dye, an azo dye, an anthraquinone dye, a perylene dye, an indigoid dye, a food, drug and cosmetic (FD&C) colorant, an organic pigment, an inorganic pigment, or a combination thereof. Examples of colorants include, but are not limited to, FD&C Red #40; Red #3; FD&C Black #3; Black #2; Mica-based pearlescent pigment; FD&C Yellow #6; Green #3; Blue #1; Blue #2; titanium dioxide (food grade); brilliant black; and a combination thereof.

In example embodiments, the water-soluble foam substrate can include any of the surfactants disclosed herein. In example embodiments, the water-soluble foam substrate can comprise one or more of the group of: sodium cocoyl isethionate, glucotain, phoenamids, cola lipid, cocamides, such as cocamide ethanolamines, ethylene oxide-based surfactants, and saponified oils of avocado and palm.

The water-soluble foam substrate of the disclosure can have any thickness. Suitable thicknesses can include, but are not limited to, about 5 microns (μm) to about 10,000 μm (1 cm), about 3 μm to about 5,000 μm, about 5 μm to about 1,000 μm, about 5 μm to about 500 μm, about 200 μm to about 500 μm, about 5 μm to about 200 μm, about 20 μm to about 100 μm, or about 40 μm to about 90 μm, or about 50 μm to 80 μm, or about or about 60 μm to 65 μm, for example, 50 μm, 65 μm, 76 μm, or 88 μm. The water-soluble foam substrate of the disclosure can be characterized as high loft or low loft. Loft refers to the ratio of thickness to mass per unit area (i.e., basis weight). High loft water-soluble foam substrates can be characterized by a high ratio of thickness to mass per unit area. As used herein, “high loft” refers to a water-soluble foam substrate of the disclosure having a basis weight as defined herein and a thickness exceeding 200 μm. The thickness of the water-soluble foam substrate can be determined according to ASTM D5729-97, ASTM D5736, and/or ISO 9073-2:1995 and can include, for example, subjecting the water-soluble foam substrate to a load of 2 N and measuring the thickness. High loft materials can be used according to known methods in the art, for example, cross-lapping, which uses a cross-lapper to fold the unbonded web over onto itself to build loft and basis weight.

The coefficient of dynamic friction and the ratio of the coefficient of static friction to the coefficient of dynamic friction for a water-soluble foam substrate of the disclosure will be lower than the coefficient of dynamic friction and the ratio of the coefficient of static friction to the coefficient of dynamic friction for a water-soluble film due to the increased surface roughness of the water-soluble foam substrate relative to a water-soluble film, which provides decreased surface contact to the water-soluble foam substrate. Advantageously, this surface roughness can provide an improved feel to the consumer (i.e., a cloth-like hand-feel instead of a rubbery hand-feel), improved aesthetics (i.e., less glossy than a water-soluble film), and/or facilitate processability which may require drawing the water-soluble foam substrate along a surface of the processing equipment/mold. Accordingly, in example embodiments, the water-soluble fibers and/or non-water-soluble fibers should be sufficiently coarse to provide a surface roughness to the resulting water-soluble foam substrate without being so coarse as to produce drag.

The solubility in water of the soluble foam substrate closure is a function of the type of fiber(s) used to prepare the water-soluble foam substrate as well as the basis weight of the water-soluble foam substrate. Without intending to be bound by theory, it is believed that the solubility profile of a water-soluble foam substrate follows the same solubility profile of the fiber(s) used to prepare the water-soluble foam substrate, and the solubility profile of the fiber generally follows the same solubility profile of the polymer(s) from which the fiber is prepared. For example, for the water-soluble foam substrates comprising PVOH fibers, the degree of hydrolysis of the PVOH polymer can be chosen such that the water-solubility of the water-soluble foam substrate is also influenced. In general, at a given temperature, as the degree of hydrolysis of the PVOH polymer increases from partially hydrolyzed (88% DH) to fully hydrolyzed (≥98% DH), water solubility of the polymer generally decreases. Thus, in example embodiments, the water-soluble foam substrate can be cold water-soluble. For a co-poly(vinyl acetate vinyl alcohol) polymer that does not include any other monomers (e.g., not copolymerized with an anionic monomer) a cold water-soluble web, soluble in water at a temperature of less than 10° C., can include fibers of PVOH with a degree of hydrolysis in a range of about 75% to about 90%, or in a range of about 75% to about 89%, or in a range of about 80% to about 90%, or in a range of about 85% to about 90%, or in a range of about 90% to about 99.5%. In other example embodiments, the water-soluble foam substrate can be hot water-soluble. For example, a co-poly(vinyl acetate vinyl alcohol) polymer that does not include any other monomers (e.g., not copolymerized with an anionic monomer), a hot water-soluble foam substrate can be soluble in water at a temperature of at least about 60° C., by including fibers of PVOH with a degree of hydrolysis of at least about 98%.

Modification of a PVOH copolymer increases the solubility of the PVOH polymer. Thus, it is expected that at a given temperature the solubility of a water-soluble foam substrate prepared from a modified PVOH copolymer would be higher than that of a water-soluble foam substrate prepared from a PVOH copolymer having the same degree of hydrolysis as the modified PVOH copolymer. Following these trends, a water-soluble foam substrate having specific solubility characteristics can be designed by blending polymers within the fibers and/or blending fibers within the water-soluble foam substrate. Further, as described herein, the water-soluble foam substrate includes a plurality of fibers that may, in some cases, include two or more fiber types that differ in solubility.

Inclusion of non-water-soluble fiber and/or non-water-soluble fiber forming material in the plurality of fibers of a water-soluble foam substrate can also be used to design a water-soluble foam substrate having specific solubility and/or prolonged release properties. Without intending to be bound by theory, it is believed that as the weight percent of non-water-soluble fiber included in a water-soluble foam substrate is increased (based on the total weight of the water-soluble foam substrate), the solubility of the water-soluble foam substrate generally decreases and the prolonged release properties of a pouch comprising a water-soluble foam substrate generally increases. Upon contact with water at a temperature at or above the solubility temperature of the water-soluble fiber, a water-soluble foam substrate comprising water-soluble fiber and non-water-soluble fiber will begin to disperse as the water-soluble fiber dissolves, thereby breaking down the foam structure and/or increasing the pore size of the pores of the water-soluble foam substrate. The larger the break-down of the foam structure or increase in the pore size, the faster the water can access the active cleansing formulation and the faster the active cleansing formulation will be released. Similarly, prolonged release of the active cleansing formulation contained in the water-soluble foam substrate of the disclosure can be achieved by using a blend of water-soluble fibers having different solubility properties and/or different solubility temperatures. Once the faster dissolving fiber has dissolved, thereby breaking up the foam, the less soluble fibers will have a larger surface area exposed, facilitating dissolution of the less soluble fibers and release of the active cleansing formulation. In example embodiments wherein the foam substrate includes water-soluble fibers and non-water-soluble fibers, the ratio of soluble fibers to non-water-soluble fibers is not particularly limited. The water-soluble fibers can comprise about 1% to about 99%, about 20% to about 80%, about 40% to about 90%, about 50% to about 90%, or about 60% to about 90% by weight, of the total weight of the plurality of fibers, and the non-water-soluble fibers can comprise about 1% to about 99%, about 20% to about 80%, about 10% to about 60%, about 10% to about 50%, or about 10% to about 40% by weight, of the total weight of the fibers. In example embodiments, the plurality of fibers comprise about 10% to about 80% water-soluble fibers by weight, based on the total weight of the fibers and the balance being non-water-soluble fibers.

In example embodiments, the nonwoven web, the plurality of fibers, the foam, the water-soluble film, or a combination thereof, disclosed herein can comprises a biodegradable polymer. In certain embodiments, the plurality of fibers can comprise non-water-soluble fiber forming materials that are biodegradable. In example embodiments, the plurality of fibers can comprise first fibers that are non-water-soluble biodegradable fibers, and second fibers that are soluble in water at a temperature of about 10° C. to about 20° C. according to MSTM-205 or not soluble in water at a temperature of about 30° C. or less according to MSTM-205, according to MSTM-205. In example embodiments, the nonwoven web is non-water-soluble and biodegradable.

In example embodiments, the water-soluble foam substrate is biodegradable. As used herein, when the water-soluble foam substrate is said to be biodegradable, at least 50% of the water-soluble foam substrate is biodegradable, for example, at least 60%, at least 70%, at least 80%, at least 90%, or 100%, of the water-soluble foam substrate is biodegradable.

The water-soluble foam substrate as disclosed herein can comprise the plurality of fibers comprising a first fiber type and a second fiber type, wherein the first and second fiber types have a difference in diameter, length, tenacity, shape, rigidness, elasticity, solubility, melting point, glass transition temperature (T_{g}), chemical composition, color, or a combination thereof. In example embodiments, the first fiber type can comprise a PVOH homopolymer fiber forming material, a PVOH copolymer fiber forming material, a modified PVOH copolymer fiber forming material, or a combination thereof. In example embodiments, the first fiber type can comprise two or more PVOH homopolymer fiber forming materials, two or more PVOH copolymer fiber forming materials, two or more modified PVOH copolymer fiber forming materials, or a combination thereof. In example embodiments, the second fiber type can comprise a PVOH homopolymer fiber forming material, a PVOH copolymer fiber forming material, a modified PVOH copolymer fiber forming material, or a combination thereof. In example embodiments, the second fiber type can comprise two or more PVOH homopolymer fiber forming materials, two or more PVOH copolymer fiber forming materials, two or more modified PVOH copolymer fiber forming materials, or a combination thereof. In example embodiments, the first fiber type and/or the second fiber type are non-water-soluble fiber forming material. In example embodiments, the first fiber type can comprise a non-water-soluble polymer fiber forming material and the second fiber type can comprise a polyvinyl alcohol fiber forming material that, when provided as the sole fiber forming material of a nonwoven web or as a film, the resulting web or film is soluble in water at a temperature in a range of about 0° C. to about 20° C. according to MSTM-205. In example embodiments, the first fiber type can comprise a non-water-soluble polymer fiber forming material and the second fiber type can comprise a PVOH copolymer or modified copolymer fiber forming material that, when provided as the sole fiber forming material of a water-soluble foam substrate, the resulting water-soluble foam substrate is not soluble in water at a temperature of 20° C. or less according to MSTM-205. In example embodiments, the first fiber type comprises two or more PVOH copolymer fiber forming materials, two or more modified PVOH copolymer fiber forming materials, or a combination of PVOH homopolymer fiber forming materials and PVOH copolymer fiber forming materials. In example embodiments, the second fiber type comprises two or more PVOH copolymer fiber forming materials, two or more modified PVOH copolymer fiber forming materials, or a combination of PVOH copolymer fiber forming materials and modified PVOH copolymer fiber forming materials.

The plurality of fibers comprised in the water-soluble foam substrate of the disclosure can have any tenacity. The tenacity of the fiber correlates to the coarseness of the fiber. As the tenacity of the fiber decreases, the coarseness of the fiber increases. Fibers used to prepare the nonwoven webs of the disclosure can have a tenacity in a range of about 1 to about 100 cN/dtex, or about 1 to about 75 cN/dtex, or about 1 to about 50 cN/dtex, or about 1 to about 45 cN/dtex, or about 1 to about 40 cN/dtex, or about 1 to about 35 cN/dtex, or about 1 to about 30 cN/dtex, or about 1 to about 25 cN/dtex, or about 1 to about 20 cN/dtex, or about 1 to about 15 cN/dtex, or about 1 to about 10 cN/dtex, or about 3 to about 8 cN/dtex, or about 4 to about 8 cN/dtex, or about 6 to about 8 cN/dtex, or about 4 to about 7 cN/dtex, or about 10 to about 20, or about 10 to about 18, or about 10 to about 16, or about 1 cN/dtex, about 2 cN/dtex, about 3 cN/dtex, about 4 cN/dtex, about 5 cN/dtex, about 6 cN/dtex, about 7 cN/dtex, about 8 cN/dtex, about 9 cN/dtex, about 10 cN/dtex, about 11 cN/dtex, about 12 cN/dtex, about 13 cN/dtex, about 14 cN/dtex, or about 15 cN/dtex. In example embodiments, the plurality of fibers can have a tenacity in a range of about 3 cN/dtex to about 15 cN/dtex, or about 5 cN/dtex to about 12 cN/dtex, or about 5 cN/dtex to about 10 cN/dtex.

The tenacity of the water-soluble foam substrate can be the same or different from the tenacity of the plurality of fibers used to prepare the web. Without intending to be bound by theory, it is believed that the tenacity of the water-soluble foam substrate is related to the strength of the nonwoven web, wherein a higher tenacity provides a higher strength to the nonwoven web. The tenacity of the water-soluble foam substrate can be modified by using fibers having different tenacities. The tenacity of the water-soluble foam substrate may also be affected by processing. The water-soluble foam substrate of the disclosure has relatively high tenacities, i.e., the water-soluble foam substrate is a self-supporting substrate that can be used as the sole material for preparing an article and/or pouch. In contrast, water-soluble foam substrate prepared according to melt blown, electro-spinning, and/or rotary spinning processes have low tenacities and may not be self-supporting or capable of being used as a sole substrate for forming an article or pouch.

Water-soluble foam substrates can be characterized by basis weight. The basis weight of a water-soluble foam substrate is the mass per unit area of the water-soluble foam substrate. Basis weight can be modified by varying manufacturing conditions, as is known in the art. A water-soluble foam substrate can have the same basis weight prior to and after bonding. Alternatively, the bonding method can change the basis weight of the water-soluble foam substrate. For example, wherein bonding occurs through the application of heat and pressure, the thickness of the water-soluble foam substrate (and, thus, the area of the water-soluble foam substrate) can be decreased, thereby increasing the basis weight. Accordingly, as used herein and unless specified otherwise, the basis weight of a water-soluble foam substrate refers to the basis weight of the water-soluble foam substrate after bonding.

The water-soluble foam substrate of the disclosure can have any basis weight in a range of about 0.1 g/m² to about 700 g/m², about 0.5 g/m² to about 600 g/m², about 1 g/m² to about 500 g/m², about 1 g/m² to about 400 g/m², about 1 g/m² to about 300 g/m², about 1 g/m² to about 200 g/m², about 1 g/m² to about 100 g/m², about 30 g/m² to about 100 g/m², about 20 g/m² to about 100 g/m², about 20 g/m² to about 80 g/m², or about 25 g/m² to about 70 g/m².

Further, as the basis weight of the water-soluble foam substrate increases, the rate of dissolution of the water-soluble foam substrate decreases, provided the fiber composition and web thickness remain constant, as there is more material to be dissolved. For example, at a given temperature, a water-soluble foam substrate prepared from fibers comprising PVOH polymer(s) and having a basis weight of, e.g., 40 g/m², is expected to dissolve slower than an otherwise-identical water-soluble web having a basis weight of, e.g., 30 g/m². Accordingly, basis weight can also be used to modify the solubility characteristics of the water-soluble foam substrate. The water-soluble foam substrate can have any basis weight in a range of about 1 g/m² to about 700 g/m², about 1 g/m² to about 600 g/m², about 1 g/m² to about 500 g/m², about 1 g/m² to about 400 g/m², about 1 g/m² to about 300 g/m², about 1 g/m² to about 200 g/m², about 10 g/m² to about 100 g/m², about 30 g/m² to about 100 g/m², about 20 g/m² to about 100 g/m², about 20 g/m² to about 80 g/m², about 25 g/m² to about 70 g/m², or about 40 g/m² to about 60 g/m².

The water-soluble foam substrate of the disclosure can be used as a single layer or can be layered with other water-soluble foam substrates or can be in the form of a laminate with a water-soluble film. In some embodiments, the water-soluble foam substrate includes a single layer. In some embodiments, the water-soluble foam substrate is a multilayer water-soluble foam substrate comprising two or more layers. The two or more layers can be laminated to each other. In refinements of the foregoing embodiment, the two or more layers can be the same (e.g., be prepared from the same fibers and basis weight). In refinements of the foregoing embodiment, the two or more layers can be different (e.g., be prepared from different types of fibers, fiber chemistries, and/or have different basis weights).

A multilayer water-soluble foam substrate can have a basis weight that is the sum of the basis weights of the individual layers. Accordingly, a multilayer water-soluble foam substrate will take longer to dissolve than any of the individual layers provided as a single layer.

### Water-Soluble Films

The water-soluble film described herein comprises any of the water-soluble polymers disclosed herein. In example embodiments, the water-soluble film of the disclosure comprises a polyvinyl alcohol (PVOH) resin, a modified polyvinyl alcohol resin, or combinations thereof. In example embodiments, the water-soluble film includes a PVOH resin selected from the group consisting of a PVOH homopolymer, a PVOH copolymer a PVOH copolymer having an anionic modification, and combinations of the foregoing. In example embodiments, the water-soluble film can comprise a single PVOH polymer or a blend of PVOH polymer. In example embodiments, the water-soluble film comprises a PVOH copolymer. In example embodiments, the water-soluble film comprises a hot water-soluble PVOH copolymer. In example embodiments wherein the nonwoven web includes a surfactant and/or an exfoliant, the water-soluble film can comprise a PVOH copolymer having an anionic modification. In example embodiments, the water-soluble film can comprise a water-soluble polyvinyl alcohol copolymer or modified copolymer that, when provided in a film as the sole film forming material, the film is soluble in water at a temperature in a range of about 0° C. to about 20° C. according to MSTM-205. In example embodiments, the water-soluble film can comprise a water-soluble polyvinyl alcohol copolymer or modified copolymer that, when provided in a film as the sole film forming material, the film is not water-soluble at a water temperature of 20° C. or less according to MSTM-205.

The water-soluble film can include other film forming polymers including, but not limited to, polyvinyl alcohols, water-soluble acrylate copolymers, polyethyleneimine, pullulan, water-soluble natural polymers including, but not limited to, guar gum, gum Acacia, xanthan gum, carrageenan, and starch, water-soluble polymer modified starches, copolymers of the foregoing, or a combination of any of the foregoing. Other water-soluble polymers can include polyalkylene oxides, polyacrylamides, celluloses, cellulose ethers, cellulose esters, cellulose amides, polyvinyl acetates, polycarboxylic acids and salts thereof, polyaminoacids, polyamides, gelatines, methylcelluloses, carboxymethylcelluloses and salts thereof, dextrins, ethylcelluloses, hydroxyethyl celluloses, hydroxypropyl methylcelluloses, maltodextrins, polymethacrylates, or a combination of any of the foregoing. Such water-soluble polymers are commercially available from a variety of sources. In example embodiments, the water-soluble film can include a PVOH homopolymer, PVOH copolymer, modified PVOH copolymer or a combination thereof. In example embodiments, the water-soluble film comprises a single PVOH copolymer or a blend of PVOH copolymers. In further embodiments, the water-soluble film comprises a PVOH copolymer with a viscosity in a range of 5 cP to 23 cP and a degree of hydrolysis in a range of 86% to 92%.

The film can have any suitable thickness, and a film thickness of about 76 microns (μm) is typical and particularly contemplated. Other values and ranges contemplated include values in a range of about 5 μm to about 200 μm, or in a range of about 20 μm to about 100 μm, or about 40 μm to about 90 μm, or about 50 μm to 80 μm, or about or about 60 μm to 65 μm, for example, 65 μm, 76 μm, or 88 μm.

In example embodiments, the water-soluble films can include an auxiliary agent as described above. In example embodiments, the water-soluble films can be substantially free of auxiliary agents as described above. In example embodiments, the water-soluble films can include a plasticizer as described above. The total amount of the non-water plasticizer provided in the water-soluble film can be in a range of about 1 wt. % to about 45 wt. %, or about 5 wt. % to about 45 wt. %, or about 10 wt. % to about 40 wt. %, or about 20 wt. % to about 30 wt. %, about 1 wt. % to about 4 wt. %, or about 1.5 wt. % to about 3.5 wt. %, or about 2.0 wt. % to about 3.0 wt. %, for example, about 1 wt. %, about 2.5 wt. %, about 5 wt. %, about 10 wt. %, about 15 wt. %, about 20 wt. %, about 25 wt. %, about 30 wt. %, about 35 wt. %, or about 40 wt. %, based on total film weight. In example embodiments, the water-soluble film comprises one or more of propylene glycol, glycerol, diglycerol, sorbitol, xylitol, maltitol, trimethylol propane (TMP), and polyethylene glycol (100-1000 molecular weight).

In example embodiments, the water-soluble films can include a surfactant as described above. In various embodiments, the amount of surfactant in the water-soluble film is in a range of about 0.01 wt. %, to about 2.5 wt. %, about 0.1 wt. % to about 2.5 wt. %, about 1.0 wt. % to about 2.0 wt. %, about 0.01 wt. % to 0.25 wt. %, or about 0.10 wt. % to 0.20 wt. %. In example embodiments, the water-soluble film comprises one or more of polysorbate 80, lecithin from various plant sources, and sodium lauryl sulfate (SLS) and the like.

In example embodiments, the auxiliary agents of the water-soluble film can include fillers/extenders/antiblocking agents/detackifying agents. Suitable fillers/extenders/antiblocking agents/detackifying agents include, but are not limited to, cross-linked polyvinylpyrrolidone, cross-linked cellulose, microcrystalline cellulose, silica, metallic oxides, calcium carbonate, talc, mica, stearic acid, and metal salts thereof, for example, magnesium stearate. Optionally, an additional unmodified starch or modified starch can be included the water-soluble in addition to one of the specific starch components described above, for example, hydroxypropylated starch present in an amount in a range of about 5 phr to about 30 phr, or modified starch having a degree of modification of greater than about 2% and is present in an amount in a range of about 2.5 phr to about 30 phr, or an unmodified starch having an amylose content in a range of about 20% to about 80%, or a hydroxypropyl modified starch having an amylose content in a range of about 23% to about 95% when the polyvinyl alcohol comprises an unmodified polyvinyl alcohol copolymer or an anionic modified polyvinyl alcohol copolymer with the proviso that the anionic modifier is not an acrylate. Preferred materials are starches, modified starches, and silica. In one embodiment, the amount of filler/extender/antiblocking agent/detackifying agent in the water-soluble film can be in a range of about 1 wt. % to about 6 wt. %, or about 1 wt. % to about 4 wt. %, or about 2 wt. % to about 4 wt. %, or about 1 phr to about 6 phr, or about 1 phr to about 4 phr, or about 2 phr to about 4 phr, for example. In example embodiments, when a starch or modified starch is included in the water-soluble film in addition to one of the specific starch components described above, the additional starch component will be provided in an amount of less than about 50 wt. %, based on the total weight of all starches included in the film. Without intending to be bound by theory, it is believed that any benefit provided to the water-soluble films of the disclosure from the inclusion of the starch component described above is not affected by including an additional starch component that provides a lesser benefit to the water-soluble film or no benefit to the water-soluble film.

The water-soluble film can further have a residual moisture content of at least 4 wt. %, for example, in a range of about 4 wt. % to about 10 wt. %, as measured by Karl Fischer titration.

### Methods of Preparing Fibers

Wet Cooled Gel Spinning

In example embodiments, the plurality of water-soluble fibers can include water-soluble fibers prepared according to a wet cooled gel spinning process, the wet cooled gel spinning process including the steps of:
(a) dissolving the water-soluble polymer (or polymers) in solution to form a polymer mixture, the polymer mixture optionally including auxiliary agents;
(b) extruding the polymer mixture through a spinneret nozzle to a solidification bath to form an extruded polymer mixture;
(c) passing the extruded polymer mixture through a solvent exchange bath;
(d) optionally wet drawing the extruded polymer mixture; and
(e) finishing the extruded polymer mixture to provide the water-soluble fibers.

The solvent in which the water-soluble polymer is dissolved can suitably be any solvent in which the water-soluble polymer is soluble. In example embodiments, the solvent in which the water-soluble polymer is dissolved includes a polar aprotic solvent. In example embodiments, the solvent in which the water-soluble polymer is dissolved includes dimethyl sulfoxide (DMSO).

The solidification bath includes a cooled solvent for gelling the extruded polymer mixture. The solidification bath can generally be at any temperature that facilitates solidification of the extruded polymer mixture. The solidification bath can be a mixture including a solvent in which the polymer is soluble and a solvent in which the polymer is not soluble. The solvent in which the polymer is not soluble is generally the primary solvent, wherein the solvent in which the polymer is not soluble makes up greater than 50% of the mixture by volume.

After passing through the solidification bath, the extruded polymer mixture gel can be passed through one or more solvent replacement baths. The solvent replacement baths are provided to replace the solvent in which the water-soluble polymer is soluble with the solvent in which the water-soluble polymer is not soluble to further solidify the extruded polymer mixture and, further, to replace the solvent in which the water-soluble polymer is soluble with a solvent that will more readily evaporate, thereby reducing the drying time. Solvent replacement baths can include a series of solvent replacement baths having a gradient of solvent in which the water-soluble polymer is soluble with the solvent in which the water-soluble polymer is not soluble, a series of solvent replacement baths having only the solvent in which the water-soluble polymer is not soluble, or a single solvent replacement bath having only the solvent in which the water-soluble polymer is not soluble. In example embodiments, at least one solvent replacement bath can consist essentially of a solvent in which the water-soluble polymer is not soluble.

Finished fibers are sometimes referred to as staple fibers, shortcut fibers, or pulp. In example embodiments, finishing includes drying the extruded polymer mixture. In example embodiments, finishing includes cutting or crimping the extruded polymer mixture to form individual fibers. Wet drawing of the extruded polymer mixture can provide a substantially uniform diameter to the extruded polymer mixture and, thus, the fibers cut therefrom. Drawing is distinct from extruding, as is well known in the art. In particular, “extruding” refers to the act of making fibers by forcing the resin mixture through the spinneret head whereas drawing refers to mechanically pulling the fibers in the machine direction to promote polymer chain orientation and crystallinity for increased fiber strength and tenacity.

In example embodiments wherein the water-soluble fibers are prepared from a wet cooled gel spinning process, the water-soluble polymer can be generally any water-soluble polymer or blend thereof, e.g., two or more different polymers, as generally described herein. In refinements of the foregoing embodiment, the polymer(s) can have any degree of polymerization (DP), for example, in a range of 10 to 10,000,000, for example, at least 10, at least 20, at least 50, at least 100, at least 200, at least 300, at least 400, at least 500, at least 750, or at least 1000 and up to 10,000,000, up to 5,000,000, up to 2,500,00, up to 1,000,000, up to 900,000, up to 750,000, up to 500,000, up to 250,000, up to 100,000, up to 90,000, up to 75,000, up to 50,000, up to 25,000, up to 12,000, up to 10,000, up to 5,000, or up to 2,500, for example in a range of 1000 to about 50,000, 1000 to about 25,000, 1000 to about 12,000, 1000 to about 5,000, 1000 to about 2,500, about 50 to about 12,000, about 50 to about 10,000, about 50 to about 5,000, about 50 to about 2,500, about 50 to about 1000, about 50 to about 900, about 100 to about 800, about 150 to about 700, about 200 to about 600, or about 250 to about 500. In example embodiments, the DP is at least 1,000. Auxiliary agents, as described above, can be added to the fibers themselves or to the nonwoven web during the carding and/or bonding process.

Thermoplastic Fiber Spinning

Thermoplastic fiber spinning is well known in the art. Briefly, thermoplastic fiber spinning includes the steps of:
(a) preparing a polymer mixture including the fiber forming polymer optionally including auxiliary agents;
(b) extruding the polymer mixture through a spinneret nozzle to form an extruded polymer mixture;
(c) optionally drawing the extruded polymer mixture; and
(d) finishing the extruded polymer mixture to provide the fibers.

The finished staple fibers of the thermoplastic fiber spinning process can be finished by drying, cutting, and/or crimping to form individual fibers. Drawing of the extruded polymer mixture mechanically pulls the fibers in the machine direction, promoting polymer chain orientation and crystallinity for increased fiber strength and tenacity. Preparing the polymer mixture for thermoplastic fiber spinning can include (a) preparing a solution of a fiber-forming material and a readily volatile solvent such that after extruding the solution through the spinneret when the solution is contacted with a stream of hot air, the solvent readily evaporates leaving solid fibers behind or (b) melting the polymer such that after extruding the hot polymer through the spinneret, the polymer solidifies by quenching with cool air. The thermoplastic fiber spinning method is distinct from the wet cooled gel spun method at least in that (a) in the thermoplastic fiber spinning method the extruded fibers are solidified by evaporation of the solvent or by quenching hot solid fibers with cool air, rather than by use of a solidification bath; and (b) in the wet-cool gel spun method, the optional drawing is performed while the fibers are in a gel state rather than a solid state.

Fiber forming materials for preparing fibers from a thermoplastic fiber spinning process can be any fiber forming polymer or blend thereof, e.g., two or more different polymers, provided that the polymer or blend thereof has suitable solubility in a readily volatile solvent and/or have a melting point lower than and distinct from their degradation temperature. Further, when a blend of fiber forming polymers are used to make a fiber, the fiber forming materials must have similar solubility in a readily volatile solvent and/or have similar heat profiles such that the two or more fiber forming materials will melt at similar temperatures. In contrast, the fiber forming materials for preparing fibers from the wet cooled gel spinning process are not as limited and fibers can be prepared from a blend of any two or more polymers that are soluble in the same solvent system, and the solvent system need not be a single solvent or even a volatile solvent.

The fiber forming polymer(s) for preparing thermoplastic fiber spun fibers can have a degree of polymerization (DP), for example, in a range of 10 to 10,000 for example, at least 10, at least 20, at least 50, at least 100, at least 200, at least 300, at least 400, at least 500, at least 750, or at least 1000 and up to 10,000, up to 5,000, up to 2,500, up to 1,000, up to 900, up to 750, up to 500, or up to 250. In example embodiments, the DP is less than 1,000.

Melt Spinning

Melt spinning is well known in the art and is understood to refer to both spun bond processes and melt blown processes. Melt spinning is a continuous process which directly prepares a nonwoven web in-line with fiber formation. As such, the melt-spun formed fibers are not finished and cut to any consistent length (e.g., staple fibers are not prepared by these processes). Additionally, melt spinning does not include a drawing step and, therefore, the only control over the diameter of the resulting melt-spun fibers is the size of the holes through which the fiber forming materials are extruded, and the polymer chains are not oriented in any specific direction.

In example embodiments, melt spinning includes the steps of:
(a) preparing a polymer mixture including the fiber forming polymer optionally including auxiliary agents;
(b) extruding the polymer mixture into a die assembly to form an extruded polymer mixture;
(c) quenching the extruded polymer mixture;
(d) depositing the quenched, extruded polymer mixture on a belt to form a nonwoven web; and
(e) bonding the nonwoven web.

In the spun bond process, the extruded polymer mixture is pumped into the die assembly as molten polymer and quenched with cold air once passed through the die assembly. In the melt blown process, the extruded polymer mixture is pumped into a die assembly having hot air blown through it and is quenched upon exiting the die assembly and coming into contact with ambient temperature air. In both processes, the fibers are continuously dropped onto a belt or drum, usually facilitated by pulling a vacuum under the belt or drum.

The diameter of melt-spun fibers are in a range of about 0.1 to about 50 micron, for example, at least about 0.1 micron, at least about 1 micron, at least about 2 micron, at least about 5 micron, at least about 10 micron, at least about 15 micron, or at least about 20 micron and up to about 50 micron, up to about 40 micron, up to about 30 micron, up to about 25 micron, up to about 20 micron, up to about 15 micron, up to about 10 micron, about 0.1 micron to about 50 micron, about 0.1 micron to about 40 micron, about 0.1 micron to about 30 micron, about 0.1 micron to about 25 micron, about 0.1 micron to about 20 micron, about 0.1 micron to about 15 micron, about 0.1 micron to about 10 micron, about 0.1 micron to about 9 micron, about 0.1 micron to about 8 micron, about 0.1 micron to about 7 micron, about 0.1 micron to about 6 micron, about 0.1 micron to about 6 micron, about 5 micron to about 35 micron, about 5 micron to about 30 micron, about 7.5 micron to about 25 micron, about 10 micron to about 25 micron, or about 15 micron to about 25 micron. It is well known in the art that melt blown processes can provide micro-fine fibers having an average diameter in a range of about 1-10 micron, however, the melt blown process has very high variation in fiber-to-fiber diameter, e.g., 100-300% variation. Further, it is well known in the art that spun bond fibers can have larger average fiber diameters, e.g., about 15 to about 25 micron, but improved uniformity between fibers, e.g., about 10% variation.

The fiber forming material for heat extruded processes (e.g., melt-spun, thermoplastic fiber spinning) is more limited than for the wet-cooled gel spun process. For example, the degree of polymerization for heat extruding processes is limited to a range of about 200 to about 500. As the degree of polymerization decreases below 200, the viscosity of the fiber forming material is too low and the individual fibers prepared by pumping the material through the die assembly do not maintain adequate separation after exiting the die assembly. Similarly, as the degree of polymerization increases above 500, the viscosity is too high to efficiently pump the material through sufficiently small holes in the die assembly to run the process at high speeds, thus losing process efficiency and fiber and/or nonwoven uniformity. Further, processes requiring heating of the fiber forming material, are unsuitable for polyvinyl alcohol homopolymers as the homopolymers generally do not have the thermal stability required.

The wet cooled gel spinning process advantageously provides one or more benefits such as providing a fiber that includes a blend of water-soluble polymers, providing control over the diameter of the fibers, providing relatively large diameter fibers, providing control over the length of the fibers, providing control over the tenacity of the fibers, providing high tenacity fibers, providing fibers from polymers having a large degree of polymerization, and/or providing fibers which can be used to provide a self-supporting nonwoven web. Continuous processes such as spun bond, melt blown, electro-spinning and rotary spinning generally do not allow for blending of water-soluble polymers (e.g., due to difficulties matching the melt index of various polymers), forming large diameter (e.g., greater than 50 micron) fibers, controlling the length of the fibers, providing high tenacity fibers, and the use of polymers having a high degree of polymerization. Further, the wet cooled gel spinning process advantageously is not limited to polymers that are only melt processable and, therefore, can access fibers made from fiber forming materials having very high molecular weights, high melting points, low melt flow index, or a combination thereof, providing fibers having stronger physical properties and different chemical functionalities compared to fibers prepared by a heat extrusion process. Further still, advantageously, the wet cooled gel spinning process is not limited by the viscosity of the polymer. In contrast, it is known in the art that processes that require melting of the fiber forming material are limited to fiber forming materials having viscosities of 5 cP or less. Thus, fibers including polymers, including polyvinyl alcohol homopolymers and copolymers, having a viscosity of greater than 5 cP are only accessible by wet cooled gel spinning.

### Methods of Preparing Nonwoven Webs

The nonwoven webs of the disclosure are sheet-like structures having two exterior surfaces, the nonwoven webs including a plurality of fibers. The nonwoven webs of the disclosures can be prepared from fibers using any known methods in the art. As is known in the art, when fibers are spun bond or melt blown, the fibers are continuously laid down to form the nonwoven web, followed by bonding of the fibers.

Staple fibers can be carded or airlaid and bonded to provide a nonwoven web. Methods of carding and airlaying are well known in the art.

Methods of bonding nonwoven webs are well known in the art. For example, bonding can include thermal, mechanical, and/or chemical bonding. Thermal bonding can include, but is not limited to calendering, embossing, air-through, and ultra-sound. Mechanical bonding can include, but is not limited to, hydro-entangling (spunlace), needle-punching, and stitch-bonding. Chemical bonding can include, but is not limited to, solvent bonding and resin bonding.

Thermal bonding is achieved by applying heat and pressure, and maintains the pore size, shape, and alignment produced by the carding process. The conditions for thermal bonding can be readily determined by one of ordinary skill in the art. If the heat and/or pressure applied is too low, the fibers will not sufficiently bind to form a free-standing web and if the heat and/or pressure is too high, the fibers will begin to meld together. The fiber chemistry dictates the upper and lower limits of heat and/or pressure for thermal bonding. Without intending to be bound by theory, it is believed that at temperatures above 235° C., polyvinyl alcohol-based fibers degrade. Methods of embossment for thermal bonding of fibers are known. The embossing can be a one-sided embossing or a double-sided embossing. Embossing of water-soluble fibers includes one-sided embossing using a single embossing roll consisting of an ordered circular array and a steel roll with a plain surface. As embossing is increased (e.g., as surface features are imparted to the web), the surface area of the web is increased. Without intending to be bound by theory it is expected that as the surface are of the web is increased, the solubility of the web is increased. Accordingly, the solubility properties of the nonwoven web can be advantageously tuned by changing the surface area through embossing.

Air-through bonding requires a high thermoplastic content in the nonwoven web and two different melting point materials. In air-through bonding, the nonbonded nonwoven web is circulated around a drum while hot air flows from the outside of the drum toward the center of the drum. Air-through bonding can provide nonwovens having low density and higher basis weight (e.g., greater than 20 to about 2000 g/m²). Nonwovens bonded by air-bonding are very soft.

Chemical bonding includes solvent bonding and resin bonding. In particular, chemical bonding may use a binder solution of a solvent and a resin (e.g., latex or the waste polymer left over from preparing the fibers). The nonwoven can be coated with the binder solution and heat and pressure applied to cure the binder and bond the nonwoven. The binder solution can be applied by immersing the nonwoven in a bath of binder solution, spraying the binder solution onto the nonwoven, extruding the binder solution onto the web (foam bonding), and/or applying the binder solution as a print or gravure.

Chemical bonding can result in smaller, less ordered pores relative to the pores as carded/melt-spun. Without intending to be bound by theory, it is believed that if the resin solution used for chemical bonding is sufficiently concentrated and/or sufficient pressure is applied, a nonporous nonwoven web can be formed. The solvent used in chemical bonding induces partial solubilization of the existing fibers in the web to weld and bond the fibers together. Thus, the solvent for chemical bonding can be any solvent that can at least partially solubilize one or more fiber forming materials of the fibers of the nonwoven. In example embodiments, the solvent is selected from the group consisting of water, ethanol, methanol, DMSO, glycerin, and a combination thereof. In example embodiments, the solvent is selected from the group consisting of water, glycerin, and a combination thereof. In example embodiments, the binder solution comprises a solvent selected from the group consisting of water, ethanol, methanol, DMSO, glycerin, and a combination thereof and further comprises a resin selected from the group consisting of polyvinyl alcohol, latex, and polyvinylpyrrolidone. The binder provided in the solution assists in the welding process to provide a more mechanically robust web. The temperature of the polymer solution is not particularly limited and can be provided at room temperature (about 23° C.).

In some embodiments, a second layer of fibers can be used to bond the nonwoven web. In example embodiments, the nonwoven layer can be bonded using thermal, mechanical, or chemical bonding, alone or in addition to bonding using an additional layer of nonwoven web/fibers.

### Methods of Laminating Films to Nonwoven Webs or Foam Substrates

Methods of preparing a laminate (e.g., water-soluble film and a nonwoven) can include, but is not limited to, calender lamination (thermal with pressure) or melt adhesion.

Calender lamination is achieved by applying heat and pressure. The conditions for calender lamination can be readily determined by one of ordinary skill in the art. In general, if the heat and/or pressure applied is too low, the fibers will not sufficiently bind to the water-soluble film to form a laminate and if the heat and/or pressure is too high, the fibers will begin to meld together with each other and the film. The fiber chemistry and film chemistry dictates the upper and lower limits of heat and/or pressure for calender lamination. Without intending to be bound by theory, it is believed that at temperatures above 235° C., polyvinyl alcohol-based fibers degrade. In example embodiments, the heat added to the overlaid nonwoven and water-soluble film is about 50° C. to about 200° C., for example, about 100° C. to about 200° C., about 110° C. to about 190° C., about 120° C. to about 180° C., or about 130° C. to about 160° C. In example embodiments, the pressure applied to the overlaid nonwoven and water-soluble film is about 5 psi to about 50 psi, such as, about 10 psi to about 40 psi, about 15 psi to about 30 psi, or about 20 psi to about 30 psi. In example embodiments, the heat added to the overlaid nonwoven and water-soluble film is about 150° C. and the pressure applied is about 25 psi. In example embodiments, the heat and pressure are applied for about 2-4 seconds. Methods of embossment for calender lamination of fibers and/or the film are contemplated. The embossing can be a one-sided embossing or a double-sided embossing. For example, embossing of water-soluble fibers and/or water-soluble films includes one-sided embossing using a single embossing roll consisting of an ordered circular array and a steel roll with a plain surface. As embossing is increased (e.g., increased amounts of surface features are imparted to the web and/or the film), the surface area of the laminate is increased. Without intending to be bound by theory it is believed that as the surface of the article is decreased, the solubility of the web and/or film is decreased. Accordingly, the solubility properties of the nonwoven web and/or water-soluble film can be advantageously tuned by changing the surface area through embossing. Without intending to be bound by theory, it is believed that as the degree of lamination of the unit dose article is increased, the surface area of the laminate decreases and the bonding between the water-soluble film and nonwoven increases, resulting in the solubility decreasing and the liquid release time increasing.

Melt adhesion lamination is achieved by applying an adhesive directly to the water-soluble film and the nonwoven web is then laid on top of the water-soluble film with the applied adhesive and is subjected to cold lamination for adhesion of the nonwoven web and the water-soluble film. As used herein, the term “cold lamination” refers to a lamination process that involves pressure but does not involve added heat. The adhesive can be any suitable adhesive to one of ordinary skill in the art. In example embodiments, the adhesive is a Henkel National Adhesive. The application of the adhesive directly to the water-soluble film can be applied by any suitable method to one of ordinary skill in the art, such as, a hot melt-spray process. In example embodiments, the melt adhesion lamination process can include a hot melt spray process at 160° C., followed by cold lamination at a pressure of 94 N/mm².

The laminate of the disclosure generally includes a water-soluble film and a nonwoven web. In example embodiments, the laminates can have a degree of lamination of about 1% to about 100%, for example, the degree of lamination can be in a range of about 1% to about 90%, or about 25% to about 75%, or about 1% to about 50%, or about 5% to about 25%, or about 25% to about 100%, or about 50% to about 100%. As used herein, the term “degree of lamination” refers to the amount of total area of the water-soluble film that is bonded to the nonwoven web. For example, a laminate having a degree of lamination of about 25% or less means that about 25% or less of the water-soluble film's area is bonded to the nonwoven web, e.g., lamination at the seals only. For example, a laminate having a degree of lamination of about 100% means that about 100% of the area of the water-soluble film is bonded to the nonwoven web. In example embodiments wherein the degree of lamination is about 25% or less, the laminate can be achieved during the heat seal process wherein the lamination occurs at each seal of the unit dose article. In example embodiments wherein the laminate has a degree of lamination of about 25% or less, this low degree of lamination can be advantageous as there is an interior void volume where the water-soluble film and the nonwoven web are not laminated providing physical separation for components having non-compatible chemistries, as well as providing an opportunity for a 2-step delivery system of compositions in a unit dose article. In example embodiments, the degree of lamination is in a range of about 5% to about 25%. In example embodiments, the degree of lamination is in a range of about 50% to about 100%.

### Dissolution and Disintegration Test (Modified MSTM-205)

A nonwoven web, water-soluble film, or laminate structure can be characterized by or tested for Dissolution Time and Disintegration Time according to the MonoSol Test Method 205 (MSTM 205), a method known in the art. See, for example, U.S. Pat. No. 7,022,656. The description provided below refers to a nonwoven web, while it is equally applicable to a water-soluble film or laminate structure.

Apparatus and Materials:

600 mL Beaker

Magnetic Stirrer (Labline Model No. 1250 or equivalent)

Magnetic Stirring Rod (5 cm)

Thermometer (0 to 100° C.±1° C.)

Template, Stainless Steel (3.8 cm×3.2 cm)

Timer (0-300 seconds, accurate to the nearest second)

Polaroid 35 mm slide Mount (or equivalent)

MonoSol 35 mm Slide Mount Holder (or equivalent)

Distilled water

For each nonwoven web to be tested, three test specimens are cut from a nonwoven web sample that is a 3.8 cm×3.2 cm specimen. Specimens should be cut from areas of web evenly spaced along the traverse direction of the web. Each test specimen is then analyzed using the following procedure.

Lock each specimen in a separate 35 mm slide mount.

Fill beaker with 500 mL of distilled water. Measure water temperature with thermometer and, if necessary, heat or cool water to maintain the temperature at the temperature for which dissolution is being determined, e.g., 20° C. (about 68° F.).

Mark height of column of water. Place magnetic stirrer on base of holder. Place beaker on magnetic stirrer, add magnetic stirring rod to beaker, turn on stirrer, and adjust stir speed until a vortex develops which is approximately one-fifth the height of the water column. Mark depth of vortex.

Secure the 35 mm slide mount in the alligator clamp of the 35 mm slide mount holder such that the long end of the slide mount is parallel to the water surface. The depth adjuster of the holder should be set so that when dropped, the end of the clamp will be 0.6 cm below the surface of the water. One of the short sides of the slide mount should be next to the side of the beaker with the other positioned directly over the center of the stirring rod such that the nonwoven web surface is perpendicular to the flow of the water.

In one motion, drop the secured slide and clamp into the water and start the timer. Rupture occurs when the sample has become compromised within the slide, for example, when a hole is created. Disintegration occurs when the nonwoven web breaks apart and no sample material is left in the slide. When all visible nonwoven web is released from the slide mount, raise the slide out of the water while continuing to monitor the solution for undissolved nonwoven web fragments. Dissolution occurs when all nonwoven web fragments are no longer visible and the solution becomes clear. Rupture and dissolution can happen concurrently for nonwoven samples wherein the fibers are prepared from polyvinyl alcohol polymers having a low degree of hydrolysis (e.g., about 65-88%). Dissolution times are recorded independently of rupture times when there is a 5 second or greater difference between rupture and dissolution.

Thinning time can also be determined using MSTM-205. Thinning of a nonwoven web occurs when some of the fibers making up the nonwoven web dissolve, while other fibers remain intact. The thinning of the web occurs prior to disintegration of the web. Thinning is characterized by a decrease in opacity, or increase in transparency, of the nonwoven web. The change from opaque to increasingly transparent and can be visually observed. During MSTM-205, after the secured slide and clamp have been dropped into the water the opacity/transparency of the nonwoven web is monitored. At the time point wherein no change in opacity/transparency is observed (i.e., the web does not become any less opaque or more transparent), the time is recorded as the thinning time.

The results should include the following: complete sample identification; individual and average disintegration and dissolution times; and water temperature at which the samples were tested.

### Method for Determining Single Fiber Solubility

The solubility of a single fiber can be characterized by the water breaking temperature. The fiber breaking temperature can be determined as follows. A load of 2 mg/dtex is put on a fiber having a fixed length of 100 mm. Water temperature starts at 1.5° C. and is then raised by 1.5° C. increments every 2 minutes until the fiber breaks. The temperature at which the fiber breaks is denoted as the water breaking temperature.

The solubility of a single fiber can also be characterized by the temperature of complete dissolution. The temperature of complete dissolution can be determined as follows. 0.2 g of fibers having a fixed length of 2 mm are added to 100 mL of water. Water temperature starts at 1.5° C. and is then raised by 1.5° C. increments every 2 minutes until the fiber completely dissolves. The sample is agitated at each temperature. The temperature at which the fiber completely dissolves in less than 30 seconds is denoted as the complete dissolution temperature.

### Diameter Test Method

The diameter of a discrete fiber or a fiber within a nonwoven web is determined by using a scanning electron microscope (SEM) or an optical microscope and an image analysis software. A magnification of 200 to 10,000 times is chosen such that the fibers are suitably enlarged for measurement. When using the SEM, the samples are sputtered with gold or a palladium compound to avoid electric charging and vibrations of the fiber in the electron beam. A manual procedure for determining the fiber diameters is used from the image (on monitor screen) taken with the SEM or the optical microscope. Using a mouse and a cursor tool, the edge of a randomly selected fiber is sought and then measured across its width (i.e., perpendicular to the fiber direction at that point) to the other edge of the fiber. A scaled and calibrated image analysis tool provides the scaling to get an actual reading in microns. For fibers within a nonwoven web, several fibers are randomly selected across the sample of nonwoven web using the SEM or the optical microscope. At least two portions of the nonwoven web material are cut and tested in this manner. Altogether at least 100 such measurements are made and then all data are recorded for statistical analysis. The recorded data are used to calculate average (mean) of the fibers, standard deviation of the fibers, and median fiber diameters.

### Tensile Strength, Modulus, and Elongation Test

A nonwoven web, water-soluble film, or laminate structure characterized by or to be tested for tensile strength according to the Tensile Strength (TS) Test, modulus (or tensile stress) according to the Modulus (MOD) Test, and elongation according to the Elongation Test is analyzed as follows. The description provided below refers to a nonwoven web, while it is equally applicable to a water-soluble film or laminate structure. The procedure includes the determination of tensile strength and the determination of modulus at 10% elongation according to ASTM D 882 (“Standard Test Method for Tensile Properties of Thin Plastic Sheeting”) or equivalent. An INSTRON tensile testing apparatus (Model 5544 Tensile Tester or equivalent) is used for the collection of nonwoven web data. A minimum of three test specimens, each cut with reliable cutting tools to ensure dimensional stability and reproducibility, are tested in the machine direction (MD) (where applicable) for each measurement. Tests are conducted in the standard laboratory atmosphere of 23±2.0° C. and 35±5% relative humidity. For tensile strength or modulus determination, 1″-wide (2.54 cm) samples of a nonwoven web are prepared. The sample is then transferred to the INSTRON tensile testing machine to proceed with testing while minimizing exposure in the 35% relative humidity environment. The tensile testing machine is prepared according to manufacturer instructions, equipped with a 500 N load cell, and calibrated. The correct grips and faces are fitted (INSTRON grips having model number 2702-032 faces, which are rubber coated and 25 mm wide, or equivalent). The samples are mounted into the tensile testing machine and analyzed to determine the 100% modulus (i.e., stress required to achieve 100% film elongation), tensile strength (i.e., stress required to break film), and elongation % (sample length at break relative to the initial sample length). In general, the higher the elongation % for a sample, the better the processability characteristics for the nonwoven web (e.g., increased formability into packets or pouches).

### Fiber Shrinkage Percent Test (MSTM)

A percent shrinkage of a fiber when contacted with a suitable amount of a carrier solvent can be determined according to a Fiber Shrinkage Percent Test under MonoSol Standard Operating Procedure.

Apparatus and Materials:

1. Fiber samples (approx. 3 grams)

2. 500 mL beaker

3. Chilled deionized water (located in refrigerator)

4. Deionized water

5. Paper clip

6. Alligator clamp (solubility stand)

7. Stir plate

8. Timer

Samples are prepared as follows:

1. Obtain a small bundle of fibers that isn't entangled. Enough to ensure it will hold in the paper clip and the alligator clamp, approximate weight of fiber bundle is 0.013 gram (g) to 0.015 g.

2. Take a paper clip and pull an end of the fiber through the cross sections of the paper clip.

3. Do this so each unique fiber to be tested has a replicate of N=3 for each testing temperature, 23° C. and 10° C.

Apparatus set-up:

1. Fill 500 ml beaker with 400 ml of respective temperature water. Make sure to check the water temperature with a temperature probe before and during testing.

2. Tape a ruler to the top of the alligator clamp so the ruler hangs parallel to the clamp.

3. Place the beaker on a stir plate and place the solubility stand next to the stir plate, submerging ruler into beaker so then you can read the length.

Testing procedure:

1. Attach the free end of the paper clipped fiber in the alligator clamp.

2. Submerge the test sample into the beaker so that the test sample is aligned next to the ruler.

3. Start the timer and record the initial length of the fiber. The test sample fiber length is from the end of alligator clip to the top of the paper clip.

4. After two minutes, record the final length of the fiber.

5. Lift the clamp out of the water and remove the sample from the clamp. Be sure to thoroughly dry off the outside of the clamp and the inside of clamp between each test.

Calculating Shrinkage Percent:

Shrinked length=initial length−final length [3]

Fiber Shrinkage (%)=(shrinked length/initial length)×100% [4]

### Uses of Skin Cleansing Articles

The skin cleansing articles of the disclosure are suitable for a variety of applications. Suitable applications for the water-dispersible or water-soluble skin cleansing articles include delivery of one or more active cleansing formulations for delivering cosmetics and/or dermal therapies to a user's skin. In example embodiments, the water-soluble core substrate has one or more areas or regions configured to contain one or more active cleansing formulations, e.g., a cosmetic or dermal therapy formulation. For example, the water-soluble core substrate may have a first region containing a first active cleansing formulation and a second region containing a second active cleansing formulation, the same or different than the first active cleansing formulation. When the water-soluble core substrate is contacted with water having a temperature greater than 20° C., or having a temperature between 30° C. and 40° C., the water-soluble core substrate is soluble to release at least one of the one or more active cleansing formulations, e.g., at least one of the first active cleansing formulation or the second active cleansing formulation. Although the water-dispersible or water-soluble skin cleansing articles are described herein as a water-dispersible or water-soluble nonwoven substrate in the form of a facial mask configured to contain one or more active cleansing formulations in one or more areas or regions of the facial mask to deliver, e.g., release, the active cleansing formulation(s) to a desired location on the skin of the user's face, the water-dispersible or water-soluble skin cleansing articles as described herein are suitable in other example embodiments for delivering active cleansing formulations or other skin wellness formulations, for example, to other locations on the skin of the user's body. Further, the water-dispersible or water-soluble skin cleansing articles may take a form other than that of a facial mask including, without limitation, a wipe, a sheet, a pad, a sachet, or a strip, for example. In example embodiments, the active cleansing formulation may include, without limitation, one or more of the following: hyaluronic acid, aloe, chamomile extract, lactic acid, citric acid, hydrolyzed collagen, a poly saccharide, a peptide, a surfactant, or a foaming agent, a soap or cleaner, a shampoo, a conditioner, a body wash, a face wash, a skin lotion, a skin treatment, a body oil, fragrance, a hair treatment, a bath salt, an essential oil, a bath bomb, an enzyme, a detergent, a surfactant, an emulsifier, a chelant, a pH adjuster, a builder, a structurant, a free fragrance, an encapsulated fragrance, a preservative, a solvent, or a mineral, and/or any ingredient suitable for including in a skin cleansing formulation, a skin wellness formulation, or a personal care formulation.

The active cleansing formulation may be in the form of a solid, e.g., a powder or a plurality of granules or particles, a gel, a liquid, or a slurry formulation, or any suitable combination of a powder, a solid, a gel, a liquid, or a slurry formulation, for example.

### EXAMPLES

### Example 1

A water-soluble facial mask having respective openings for the user's eyes, nose, and mouth, includes a water-soluble nonwoven substrate having a basis weight of 30 gsm to 80 gsm, including water-soluble fibers made of a carded and calendered process. The water-soluble nonwoven substrate includes or contains an active cleansing formulation adjusted to a pH of 3.8 to 4.5 as described in Table 1 below:

TABLE 1
Example Active Cleansing Formulation

| Deionized Water | <10 wt. % |
|---|---|
| Sodium Benzoate | 0.30 |
| Potassium Sorbate | 0.30 |
| Glycolic Acid | 3.03 |
| Sodium Hydroxide | 0.66 |
| Allantoin | 0.10 |
| Sodium Hyaluronate | 0.10 |
| Ectoin | 0.10 |
| Disodium Lauryl | |
| Sulfosuccinate | 8.00 |
| | 100.00 |

The active cleansing formulation is provided in an aqueous solution. The aqueous solution is coated on at least one planar surface of the water-soluble nonwoven substrate using any suitable coating technique known to those having ordinary skill in the art, such as a Mayer roll, slot die, die, curtain, gravure, kiss roller, or dip coating technique. Alternatively, the active cleansing formulation is supplied in a dry, solid phase or in a slurry phase, and can be applied to the water-soluble nonwoven substrate using any suitable application technique known to those having ordinary skill in the art for supplying a solid formulation, such as an air sprayed, blasted, or tumbled technique.

The nonwoven substrate with the active cleansing formulation applied to at least one surface is then cut into a shape of a facial mask using any suitable cutting technique known to those having ordinary skill in the art, such as a die cutting technique. The facial mask is then packaged in a dry state, i.e., containing no water or substantially free of water, e.g., containing an insignificant amount of water, and placed in a recyclable package, as shown in FIG. 4, for example. In example embodiments, a plurality of facial masks, e.g., 10 facial masks, 25 facial masks, or 50 facial masks, are packaged in a recyclable package.

In example embodiments, the facial mask is initially provided in a dry, stable state (i.e., requiring significantly reduced secondary packaging) and water is added to the facial mask before or during use. Providing the facial mask in a dry state may assist with or be beneficial to regulatory and/or product testing concerns regarding, for example, concentration and/or pH of various materials or components of the active cleansing formulation, e.g., glycolic acid or sodium hyaluronate, which may be regulated as a skin irritant.

Example 1 as described herein is a general example. The active cleansing formulation in Table 1 and the nonwoven substrate as one example of the core substrate are described for illustration purposes only. The core substrate and the active cleansing formulation can have any suitable composition and/or any suitable form as described herein. For example, the core substrate can include a water-dispersible or water-soluble nonwoven, foam, or film, or any combination thereof. Such core substrate may include one or more PVOH polymers, such as vinyl alcohol-vinyl acetate copolymer. For example, in certain embodiments, the core substrate includes at least one nonwoven web, sheet, or layer comprising the plurality of fibers. The plurality of fibers comprise a first type of fiber comprising a polyvinyl alcohol copolymer having a degree of hydrolysis in a range of about 75% to about 89%, and a second type of fiber comprising a polyvinyl alcohol copolymer having a degree of hydrolysis in a range of about 90% to about 99.5%. The first type of fiber and the second type of fiber are at a suitable ratio, for example, in a range of from about 25:75 to about 95:5 or from about 25:75 to about 75:25 by weight. In certain embodiments, the first type of fiber and the second type of fiber are mixed together in the at least one nonwoven sheet or layer. In certain embodiments, the at least one nonwoven sheet or layer comprises a first type of nonwoven sheet or layer made of the first type of fiber, and a second type of nonwoven sheet or layer made of the second type of fiber, i.e., the two types of fibers are in different nonwoven sheets.

### Fibers Used

As shown in Table 2, two types of fibers, Fiber 1 (“F1”) and Fiber 2 (“F2”), which comprise a copolymer of vinyl acetate and vinyl alcohol having a degree of hydrolysis of 88% and 96%, respectively, were used as the starting materials. These fibers have uniform composition, and have additional properties shown in Table 2. In the Examples described herein, the fibers being used have a fineness of 2.2 dtex. In the Examples, a polymer comprising vinyl alcohol moieties is referred as “a polyvinyl alcohol polymer,” and a fiber comprising such a polymer is referred as “a polyvinyl alcohol fiber.” The units of the fineness dtex and dpf are close to each other and can be converted using a coefficient (dtex=dpf/0.9).

TABLE 2

| | Viscosity | DH | Fineness | Solubility | Tenacity | Elongation |
|---|---|---|---|---|---|---|
| Fiber | (4% solution) | (mol %) | (dtex) | Temp (C) | (cN/dtex) | (%) |
| F1 | 22-23 | 88 | 2.2 | 20 | 5 | 20 |
| F2 | 22-23 | 96 | 2.2 | 40 | 7 | 15 |

As shown in Table 3, under different bonding conditions, the two types of fibers were used to make nonwoven core substrates. The two types of fibers were used to make separate and different nonwoven layers, which were then used to make the multiple-layer nonwoven core substrate. The “multi-layer nonwoven core substrate” samples refer to those having different sheets made of different fiber compositions. The two types of fibers were also mixed to make one nonwoven layer as the core substrate (referred to as a “blended nonwoven core substrate”). The “blended nonwoven core substrate” refers to a core substrate having a same formulation but different fibers in one sheet. Multiple of layers or sheets of the blended nonwoven can also be used. Before the calender bonding process, a pre-needled punching was applied to some samples in order to increase bonding in the beginning.

TABLE 3

| | | Composition | | | | | | | |
|---|---|---|---|---|---|---|---|---|---|
| | | Fiber | Fiber | Bonding Conditions | | | | | |
| | | F1 - 2.2 | F2 - 2.2 | Pre- | Calender | Calender | Calender | Basis weight | |
| | | dpf × 51 mm | dpf × 51 mm | Needled | Speed | Pressure | Temp. | Target | Measured |
| Sample/Substrate | | (gsm or wt. %) | (gsm or wt. %) | Punched | (FPM) | (PSI) | (° C.) | (GSM) | (GSM) |
| Multi- | M1-A | 20, 20 | | No | 2 | 40 | 140 | 40 | |
| Layer | M1-B | 20, 20 | | Yes | 2 | 40 | 140 | 40 | |
| Nonwovens | M2-A | | 20, 20 | No | 2 | 40 | 140 | 40 | |
| (Composition | M2-B | | 20, 20 | Yes | 2 | 40 | 140 | 40 | |
| in | M3-A | 20 | 20 | No | 2 | 40 | 140 | 40 | |
| gsm) | M3-B | 20 | 20 | Yes | 2 | 40 | 140 | 40 | |
| | M6-A | 30 | 30 | Yes | 2 | 40 | 150 | 60 | |
| | M6-B | 30 | 30 | No | 2 | 40 | 150 | 60 | |
| Blended | C5-A | 100 | 0 | No | 40 | 40 | 140 | 40 | 40.1 |
| Nonwovens | C6-A | 0 | 100 | No | 40 | 40 | 140 | 40 | 42.5 |
| (Composition | B1-A | 25 | 75 | No | 40 | 40 | 140 | 40 | 47.9 |
| in | B2-A | 50 | 50 | No | 40 | 40 | 140 | 40 | 43.8 |
| wt. %) | B3-A | 75 | 25 | No | 40 | 40 | 140 | 40 | 32.9 |

For each substrate in Table 3, two types of samples, including the untreated nonwovens and the nonwovens treated (loaded) with the active cleansing formulation (“post active nonwovens”), were tested for solubility, at 23° C. and 40° C., respectively. Tables 4 and 5 show the solubility results of the untreated samples (substrates only) and the post-active samples at 23° C. and 40° C., respectively. Table 5 also shows tensile testing results of these water soluble substrates. For each sample, multiple specimen were tested. For the purpose of conciseness, the standard deviation values of the data were not shown.

TABLE 4

| | | Composition | | | | | | |
|---|---|---|---|---|---|---|---|---|
| | | Fiber | Fiber | Pre- | | | Solubility 23° C. | |
| | | F1 (gsm | F2 (gsm | Needled | Solubility 23° C. | | Post Active Exposure | |
| Sample Substrate | | or wt. %) | or wt. %) | Punched | Rupture (s) | Disintegration (s) | Rupture (s) | Disintegration (s) |
| Multi- | M1-A | 20, 20 | | No | 18.7 | 46.7 | 16.3 | 34.3 |
| Layer | M1-B | 20, 20 | | Yes | 23.0 | 38.7 | 18.7 | 35.3 |
| Nonwovens | M2-A | | 20, 20 | No | 103.3 | 300+ | 75.7 | 136.7 |
| (gsm) | M2-B | | 20, 20 | Yes | 108.3 | 300+ | 77.3 | 143.3 |
| | M3-A | 20 | 20 | No | 101.0 | 300+ | 73.3 | 144.7 |
| | M3-B | 20 | 20 | Yes | 94.7 | 300+ | 70.3 | 142.7 |
| | M6-A | 30 | 30 | Yes | 104.9 | 300+ | 76.3 | 155.3 |
| | M6-B | 30 | 30 | No | 102.2 | 300+ | 73.3 | 159.3 |
| Blended | C5-A | 100 | 0 | No | 24.7 | 42.7 | 20.3 | 36.3 |
| Nonwovens | C6-A | 0 | 100 | No | 138.7 | 209.0 | 87.7 | 166.3 |
| (wt. %) | B1-A | 25 | 75 | No | 99.0 | 134.7 | 63.7 | 145.0 |
| | B2-A | 50 | 50 | No | 101.0 | 146.7 | 62.3 | 132.3 |
| | B3-A | 75 | 25 | No | 69.0 | 122.7 | 37.3 | 107.7 |

TABLE 5

| | | Composition | | | | | | | |
|---|---|---|---|---|---|---|---|---|---|
| | | Fiber | Fiber | Pre- | | | Solubility 40° C. | | Tensile |
| | | F1 (gsm | F2 (gsm | Needled | Solubility 40° C. | | Post Active Exposure | | Max |
| Sample/Substrate | | or wt. %) | or wt. %) | Punched | Rupture (s) | Disintegration (s) | Rupture (s) | Disintegration (s) | Load (N) |
| Multi- | M1-A | 20, 20 | | No | 2.3 | 4.0 | 2.1 | 3.6 | 9.3 |
| Layer | M1-B | 20, 20 | | Yes | 3.3 | 17.0 | 2.5 | 5.7 | 9.8 |
| Nonwovens | M2-A | | 20, 20 | No | 7.3 | 17.4 | 6.3 | 15.3 | 5.2 |
| (gsm) | M2-B | | 20, 20 | Yes | 5.7 | 15.7 | 5.5 | 13.7 | 4.7 |
| | M3-A | 20 | 20 | No | 6.3 | 16.0 | 5.5 | 14.3 | 7.3 |
| | M3-B | 20 | 20 | Yes | 5.0 | 14.3 | 4.7 | 13.3 | 10.0 |
| | M6-A | 30 | 30 | Yes | 7.0 | 17.7 | 5.8 | 15.3 | 8.2 |
| | M6-B | 30 | 30 | No | 6.0 | 17.2 | 5.2 | 15.8 | 7.9 |
| Blended | C5-A | 100 | 0 | No | 3.0 | 7.0 | 2.8 | 6.7 | 7.9 |
| Nonwovens | C6-A | 0 | 100 | No | 11.0 | 20.3 | 9.7 | 19.0 | 6.2 |
| (wt. %) | B1-A | 25 | 75 | No | 7.0 | 19.0 | 7.3 | 18.7 | 8.6 |
| | B2-A | 50 | 50 | No | 5.3 | 9.7 | 4.5 | 9.7 | 9.3 |
| | B3-A | 75 | 25 | No | 5.7 | 12.7 | 4.8 | 10.3 | 8.4 |

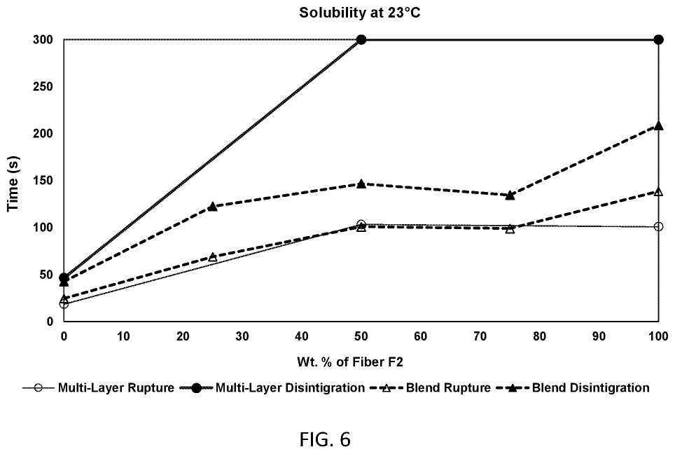

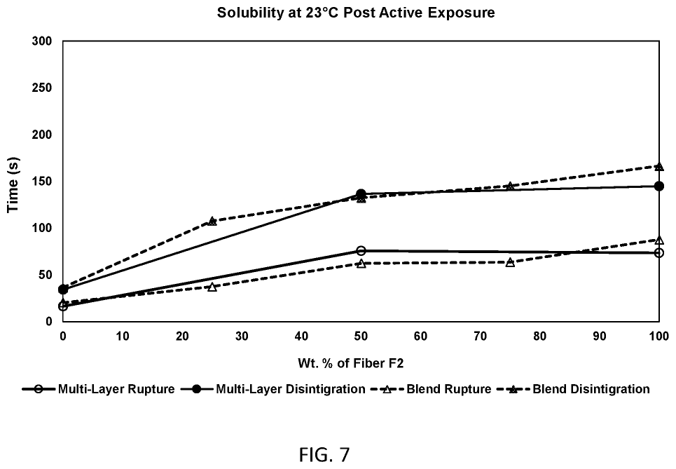

FIG. 6 shows the solubility results (with water at 23° C.) of core substrates comprising at least one nonwoven layer or sheet having the plurality of fibers, which include a first type of fiber (“F1”) comprising a polyvinyl alcohol copolymer having a degree of hydrolysis of 88% and a second type of fiber (“F2”) comprising a polyvinyl alcohol copolymer having a degree of hydrolysis of 96%. FIG. 7 shows the solubility results (at 23° C.) of the samples including the core substrates in FIG. 6 treated with an active cleansing formulation. At 23° C., the multi-layer and the blended water-soluble nonwoven sheets or layers have similar rupture time. The solubility generally decreases with an increase in the content of Fiber F2 having a higher degree of hydrolysis. Blended nonwovens tend to disintegrate much faster than the multi-layer nonwovens. For example, when the content of Fiber F2 is 50%, the multi-layer nonwoven has a homogenous web made of Fiber F2, while the blended nonwoven is more heterogeneous, resulting in faster disintegration. The samples of nonwoven substrates with an active cleansing formulation having a low-water content show significantly faster disintegration and solubility after such exposure. The blended nonwovens and the multi-layer nonwovens after exposure show similar disintegration times. The active cleansing formulation contains a polyol carrier, which may loosen the structure of the nonwoven substrates.

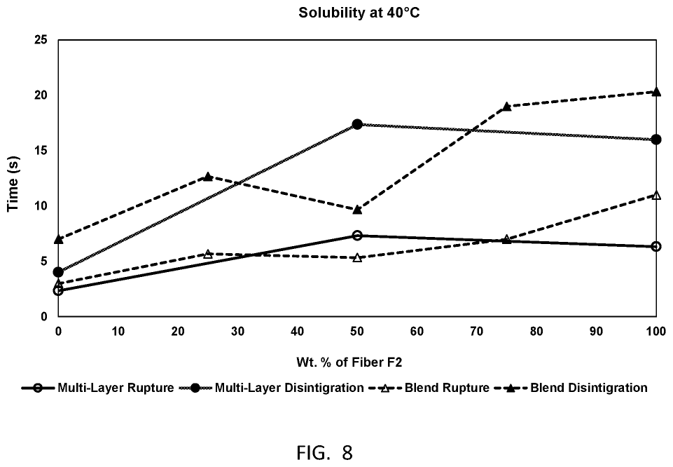

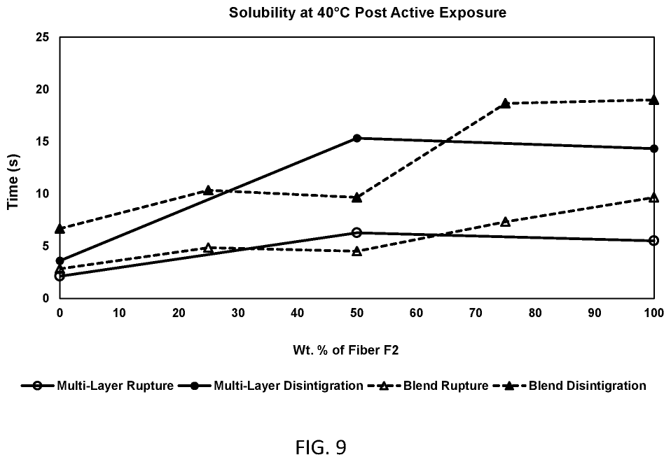

FIGS. 8 and 9 show the solubility results of the same samples as shown in FIGS. 6 and 7, except that the testing temperature is 40° C. The blended nonwovens and the multi-layer nonwovens show similar rupture and disintegration times. With the increase in the content of Fiber F2 having a higher degree of hydrolysis, the solubility decreases, but at a slower rate compared to the data at 23° C. At 40° C., the exposure to the active cleansing formulation does not have a significant impact on disintegration and solubility of the nonwoven samples.

Based on the results shown in FIGS. 6-9, the first type of fiber (F1) and the second type of fiber (F2) may, in example embodiments, be at a ratio of 25:75 or higher, for example, in a range of from about 25:75 to about 95:5, about 25:75 to about 85:15, or about 25:75 to about 75:25, by weight. For example, when only Fiber F1 is used or the content of Fiber F1 is too high, the nonwoven substrate may disintegrate too fast before a resulting skin cleaning article is applied. With a higher content of Fiber F2, a resulting skin cleaning article may disintegrate or dissolve in a desirable period of time, and can be washed off from the skin after application.

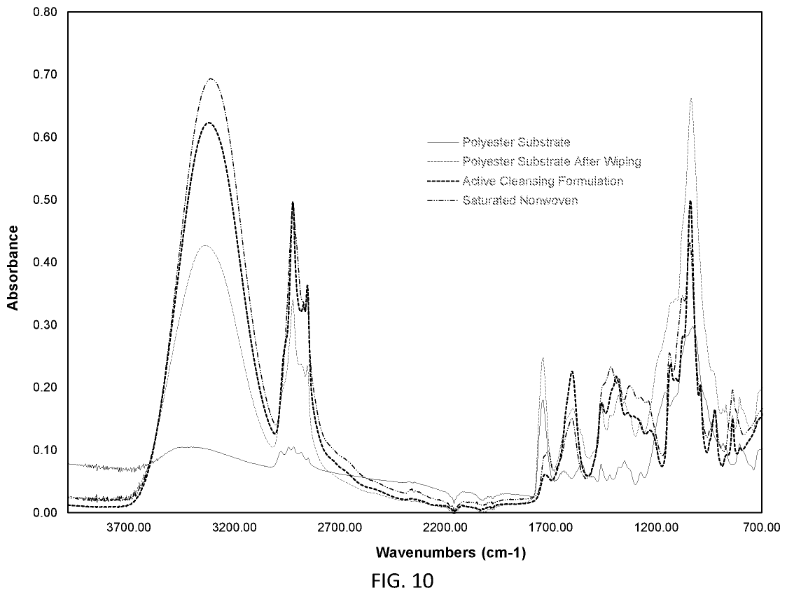

FIG. 10 shows FI-IR curves illustrating transfer of an active cleansing formulation from a water-soluble nonwoven core substrate to a surface of a separate object made of polyester. A nonwoven substrate made of a PVOH copolymer was cut into a square of about 2.5 cm×2.5 cm, and a detergent as an example of an active cleansing formulation was poured onto the nonwoven substrate. After a 10-minute soaking, excessive detergent on the surface of the substrate was blotted off. The nonwoven substrate was then used to wipe the polyester surface. Attenuated total reflectance (ATR) FT-IR was used to scan each sample surface. Three FT-IR curves were obtained from a nonwoven sample having the active cleaning formulation, a polyester surface, and the polyester surface after exposed to the nonwoven samples having the active formulation, compared to that of the active cleaning formulation. The intensity increase of peaks at 3325 cm⁻¹ and 1600 cm⁻¹, corresponding to hydroxyl and carbonyl groups, respectively, indicated that the active cleansing formulation was transferred from the water-soluble nonwoven sample to the polyester surface.

Example embodiments of the disclosure are described in the following numbered paragraphs. These example embodiments are intended to be illustrative in nature and not intended to be limiting.

The following paragraphs describe further aspects of the disclosure:

1. A skin cleansing article configured to deliver cosmetics or dermal therapies to a user's skin, the skin cleansing article comprising:

a core substrate comprising a resin, the core substrate having a first region containing a first active cleansing formulation and a second region containing a second active cleansing formulation,

wherein the skin cleansing article issonfigured to be at least water-dispersible or water-soluble, and the core substrate is at least dispersible or soluble upon contact with water having a temperature greater than 10° C. for a period of time according to Testing Method MSTM-205 so as to release at least one of the first active cleansing formulation and the second active cleansing formulation.

2. The skin cleansing article according to clause 1, wherein the skin cleansing article is substantially dry or solid with a content of moisture or solvent less than 10 wt. % before contacting with water.

3. The skin cleansing article according to clause 1 or 2, wherein the period of time is in a range of from about 30 seconds to about 300 seconds, or from about 30 seconds to about 600 seconds, or from about 30 seconds to about 900 seconds.

4. The skin cleansing article according to any of clauses 1-3, wherein, at a temperature of 30° C. to 40° C., the core substrate has a dispersion time or a dissolution time of 300 seconds or less according to MSTM-205.

5. The skin cleansing article according to any of clauses 1-4, wherein the core substrate is substantially planar and formable to a contour of a surface of the user's skin.

6. The skin cleansing article according to any of clauses 1-5, wherein the core substrate comprises at least one nonwoven substrate including a plurality of fibers comprising the resin selected from at least one of a water-dispersible resin or a water-soluble resin.

7. The skin cleansing article according to clause 6, wherein the resin is a polymer comprising a vinyl alcohol moiety.

8. The skin cleansing article according to clause 7, wherein the vinyl alcohol moiety comprises a polyvinyl alcohol homopolymer, a polyvinyl alcohol copolymer, or a combination thereof.

9. The skin cleansing article according to clause 8, wherein the polyvinyl alcohol copolymer is a copolymer of vinyl acetate and vinyl alcohol.

10. The skin cleansing article according to clause 8 or 9, wherein the polyvinyl alcohol copolymer comprises an anionically modified copolymer.

11. The cleansing article according to clause 10, wherein the anionically modified copolymer comprises a carboxylate, a sulfonate, or combinations thereof.

12. The cleansing article according to any of clauses 8-11, wherein the plurality of fibers comprise a first type of fiber comprising a polyvinyl alcohol copolymer having a degree of hydrolysis in a range of about 75% to about 89%, and a second type of fiber comprising a polyvinyl alcohol copolymer having a degree of hydrolysis in a range of about 90% to about 99.5%.

13. The cleansing article according to clause 12, wherein a ratio of the first type of fiber to the second type of fiber is in a range of from about 25:75 to about 75:25 by weight.

14. The cleansing article according to clause 12 or 13, wherein the first type of fiber and the second type of fiber are mixed together in the at least one nonwoven sheet.

15. The cleansing article according to clause 14, wherein the at least one nonwoven sheet comprises a first nonwoven layer made of the first type of fiber and a second nonwoven layer made of the second type of fiber.

16. The skin cleansing article according to any of clauses 6-15, wherein the plurality of fibers comprise 10% to 80% water-soluble fibers by weight based on a total weight of the plurality of fibers and a balance being non-water-soluble fibers.

17. The skin cleansing article according to any of clauses 1-16, wherein the first region is positionable at a first location on a face of the user and the second region is positionable at a second location on the face of the user.

18. The skin cleansing article according to any of clauses 8-17, wherein the first region is separable from the second region.

19. The skin cleansing article according to any of clauses 1-18, wherein each of the first active cleansing formulation and the second active cleansing formulation is in the form of at least one of the following: a solid, a liquid, a gel, or a slurry form.

20. The skin cleansing article according to any of clauses 1-19, wherein at least one of the first active cleansing formulation or the second active cleansing formulation is in the solid form having a moisture content of less than 10%.

21. The skin cleansing article according to any of clauses 1-20, wherein each of the first active cleansing formulation and the second active cleansing formulation comprises one or more of the following: hyaluronic acid, aloe, chamomile extract, lactic acid, citric acid, hydrolyzed collagen, a poly saccharide, a peptide, a surfactant, a foaming agent, ceramides, glycolic acid, an alpha-hydroxy acid, an amino acid, a activated carbon, sunscreen, minerals (Zn), avobenzone, antioxidants, energizers, caffeine, ginsing, taurine, retinol, retinoic acid, Niacinamide, salicyclic acid, lactic acid, or aseliaic acid, or any combination thereof.

22. The skin cleansing article according to any of clauses 1-21, wherein each of the first active cleansing formulation and the second active cleansing formulation is at least one of disposed on a surface of the core substrate or embedded in a matrix of the core substrate.

23. The skin cleansing article according to any of clauses 1-22, wherein the core substrate is at least one of saturated with an active cleansing formulation, coated with an active cleansing formulation, or impregnated with an active cleansing formulation.

24. The skin cleansing article according to any of clauses 1-23, wherein the core substrate transitions into a hydrogel upon contact with water for 300 seconds or less at 10° C. or above.

25. A facial mask configured to deliver cosmetics or dermal therapies to a user's skin, the facial mask, comprising:

a nonwoven substrate including a plurality of fibers comprising a water-soluble resin, the nonwoven substrate being water-soluble and having a first region and a second region;

a first active cleansing formulation contained in the first region; and

a second active cleansing formulation contained in the second region,

wherein the nonwoven substrate is soluble upon contact with water having a temperature greater than 10° C. for 300 seconds or less according to Testing Method MSTM-205 so as to release at least one of the first active cleansing formulation and the second active cleansing formulation from the water-soluble nonwoven substrate.

26. The facial mask according to clause 25, wherein, at a temperature of 30° C. to 40° C., the nonwoven substrate has a dissolution time of 300 seconds or less according to MSTM-205.

27. The facial mask according to clause 25 or 26, wherein the nonwoven substrate has a moisture content of less than 10%.

28. The facial mask according to any of clauses 25-27, wherein the nonwoven substrate is substantially planar and formable to a contour of a surface of the user's skin.

29. The facial mask according to any of clauses 25-28, wherein the first region is positionable at a first location on a face of the user and the second region is positionable at a second location on the face of the user, wherein the first region is separate from the second region.

30. The mask according to any of clauses 25-29, wherein each of the first active cleansing formulation and the second active cleansing formulation is in the form of at least one of the following: a solid, a liquid, a gel, or a slurry form.

31. The facial mask according to any of clauses 25-30, wherein each of the first active cleansing formulation and the second active cleansing formulation comprises one or more of the following: hyaluronic acid, aloe, chamomile extract, lactic acid, citric acid, hydrolyzed collagen, a poly saccharide, a peptide, a surfactant, a foaming agent, ceramides, glycolic acid, an alpha-hydroxy acid, an amino acid, a activated carbon, sunscreen, minerals (Zn), avobenzone, antioxidants, energizers, caffeine, ginsing, taurine, retinol, retinoic acid, Niacinamide, salicyclic acid, lactic acid, or aseliaic acid, or any combination thereof.

32. The facial mask according to any of clauses 25-31, wherein the plurality of fibers are saturated with one of the first active cleansing formulation or the second active cleansing formulation.

33. The facial mask according to any of clauses 25-32, wherein each of the first active cleansing formulation and the second active cleansing formulation is one of disposed on a surface of the plurality of fibers or embedded in the plurality of fibers.

34. The facial mask according to any of clauses 25-33, wherein the plurality of fibers comprise a fiber type including one or more of the following: a polyvinyl alcohol homopolymer, a polyvinyl alcohol copolymer, or a combination thereof.

35. The facial mask according to clause 34, wherein the polyvinyl alcohol copolymer has a degree of hydrolysis in a range of about 75% to about 89%.

36. The facial mask according to clause 34, wherein the polyvinyl alcohol copolymer has a degree of hydrolysis in a range of about 90% to about 99.9%.

37. The facial mask according to any of clauses 34-36, wherein the polyvinyl alcohol copolymer comprises an anionically modified copolymer comprising a carboxylate, a sulfonate, or combinations thereof.

38. The facial mask according to any of clauses 25-37, wherein the plurality of fibers comprise a first type of fiber and a second type of fiber, wherein the first type of fiber and the second type of fiber have a difference in one or more of the following characteristics: a length to diameter (LID) ratio, atenacity, a shape, a rigidness, an elasticity, a solubility in water, a melting point, a glass transition temperature (T₉), a fiber chemical composition, or a color, or any combination thereof.

39. The facial mask according to clause 38, wherein each of the first type of fiber and the second type of fiber comprises a polyvinyl alcohol homopolymer, a polyvinyl alcohol copolymer, or a combination thereof.

40. The facial mask according to clause 38 or 39, wherein the first type of fiber comprises a polyvinyl alcohol copolymer having a degree of hydrolysis in a range of 75% to 89%, and

the second type of fiber comprises a polyvinyl alcohol copolymer having a degree of hydrolysis in a range of 90% to 99.5%.

41. The facial mask according to clause 40, wherein a ratio of the first type of fiber to the second type of fiber is in a range of from about 25:75 to about 75:25 by weight.

42. The facial mask according to clause 40 or 41, wherein the first type of fiber and the second type of fiber are mixed together in a same nonwoven sheet or separated in different nonwoven sheets.

43. The facial mask according to any of clauses 38-42, wherein one of the first type of fiber or the second type of fiber comprises a non-water-soluble polymer fiber forming material.

44. The facial mask according to clause 43, wherein the non-water-soluble polymer fiber-forming material comprises one or more of the following materials: cotton, hemp, jute, flax, ramie, sisal, bagasse, banana, lacebark, silk, sinew, catgut, wool, sea silk, mohair, angora, cashmere, collagen, actin, nylon, dacron, rayon, bamboo, modal, cellulose diacetate, cellulose triacetate, or combinations thereof.

45. The facial mask according to any of clauses 25-44, wherein the water-soluble nonwoven substrate has a linear mass density in a range of 1 dtex to 5 dtex.

46. The facial mask according to any of clauses 25-45, wherein the water-soluble nonwoven substrate is biodegradable.

47. The facial mask according to any of clauses 25-46, wherein the plurality of fibers comprise at least one fiber type having a tenacity in a range of 3 cN/dtex to 15 cN/dtex,

having a mean diameter in a range of 10 microns to 300 microns,

having substantially uniform mean diameters,

and/or having an average length in a range of 10 millimeters (mm) to 100 mm.

48. The facial mask according to any of clauses 25-47, wherein the nonwoven substrate has a porosity of 30% to 90%.

49. The facial mask according to clause 27, wherein the water-soluble resin comprises a polyvinyl alcohol copolymer having a degree of hydrolysis in a range of 75% to 99.9%.

50. The facial mask according to any of clauses 25-49, wherein upon contact with water having a temperature greater than 10° C., the nonwoven substrate exhibits a degree of shrinkage of 0.5% to 65%.

51. A method for making a skin cleansing article, the method comprising:

forming a core substrate comprising a water-soluble resin, the water-soluble nonwoven substrate having a first region and a second region; and

containing a first active cleansing formulation in the first region and containing a second active cleansing formulation in the second region.

52. The method according to clause 51, wherein containing a first active cleansing formulation in the first region comprises at least one of saturating the first region of the core substrate with the first active cleansing formulation, disposing the first active cleansing formulation on a surface of the first region of the core substrate, coating a surface of the first region of the core substrate with the first active cleansing formulation, embedding the first active cleansing formulation in the first region of the core substrate, or impregnating the first region of the core substrate with the first active cleansing formulation.

53. The method according to clause 51 or 52, wherein forming the core substrate comprising a resin comprises recycling the core substrate to produce the resin.

54. The method according to any of clauses 51-53, wherein the resin and the core substrate are water-soluble, and the skin cleansing article is a water-soluble facial mask.

55. A skin cleansing article configured to deliver cosmetics or dermal therapies to a user's skin, the skin cleansing article comprising:

a core substrate comprising a resin, the core substrate having a first region containing a first active cleansing formulation, wherein the skin cleansing article is substantially dry or solid, and is configured to be at least water-dispersible or water-soluble,

wherein the core substrate is at least dispersible or soluble upon contact with water having a temperature greater than 10° C. for a period of time according to Testing Method MSTM-205 so as to release the first active cleansing formulation from the water-dispersible core substrate.

56. The skin cleansing article according to clause 55, wherein the temperature is greater than 40° C. and the period of time is from 30 seconds to 300 seconds.

57. A skin cleansing article configured to deliver cosmetics or dermal therapies to a user's skin, comprising:

a first nonwoven substrate including a plurality of fibers comprising a water-soluble resin or a water-dispersible resin, the first nonwoven substrate having a first region;

a first active cleansing formulation contained in the first region;

a second nonwoven substrate coupled to the first nonwoven substrate, the second nonwoven substrate including a plurality of fibers comprising one of a water-dispersible resin or a water-soluble resin, the second nonwoven substrate having a second region; and

a second active cleansing formulation contained in the second region,

wherein, when the first nonwoven substrate is contacted with water having a temperature greater than 10° C. for 300 seconds or less, the first nonwoven substrate is soluble or dispersible according to MSTM-205 to release the first active cleansing formulation from the first nonwoven substrate.

58. The skin cleansing article according to clause 57, wherein the first nonwoven substrate is water soluble, the second nonwoven substrate includes a plurality of fibers comprising a water-dispersible resin, and, when the second nonwoven substrate is contacted with water having a temperature greater than 10° C. for 300 seconds or less, the second nonwoven substrate is dispersible according to MSTM-205 to release the second active cleansing formulation from the second nonwoven substrate.

59. The skin cleansing article according to clause 57 or 58, wherein the first nonwoven is water soluble, the second nonwoven substrate includes a plurality of fibers comprising a water-soluble resin, and, when the second nonwoven substrate is contacted with water having a temperature greater than 10° C. for 300 seconds or less, the second nonwoven substrate is soluble according to MSTM-205 to release the second active cleansing formulation from the second nonwoven substrate.

60. The skin cleansing article according to any of clauses 57-59, further comprising a water-soluble or water dispersible film coupled to one of the first nonwoven substrate and the second nonwoven substrate.

All percentages, parts and ratios referred to herein are based upon the total dry weight of the fiber composition, film composition, or total weight of the packaging material composition of the present disclosure, as the case may be, and all measurements made are at about 25° C., unless otherwise specified. All percentages, parts and ratios referred to herein for liquid formulations are based upon the total weight of the liquid formulation. All such weights as they pertain to listed ingredients are based on the active level and therefore do not include carriers or by-products that may be included in commercially available materials, unless otherwise specified.

All ranges set forth herein include all possible subsets of ranges and any combinations of such subset ranges. By default, ranges are inclusive of the stated endpoints, unless stated otherwise. Where a range of values is provided, it is understood that each intervening value between the upper and lower limit of that range and any other stated or intervening value in that stated range, is encompassed within the disclosure. The upper and lower limits of these smaller ranges may independently be included in the smaller ranges, and are also encompassed within the disclosure, subject to any specifically excluded limit in the stated range. Where the stated range includes one or both of the limits, ranges excluding either or both of those included limits are also contemplated to be part of the disclosure.

It is expressly contemplated that for any number value described herein, e.g., as a parameter of the subject matter described or part of a range associated with the subject matter described, an alternative which forms part of the description is a functionally equivalent range surrounding the specific numerical value (e.g., for a dimension disclosed as “40 millimeters (mm)” an alternative embodiment contemplated is “about 40 mm”).

Reference throughout this specification to “example embodiment” or “an embodiment” may mean that a particular feature, structure, or characteristic described in connection with a particular embodiment may be included in at least one embodiment of claimed subject matter. Thus, appearances of the phrase “example embodiments” or “an example embodiment” in various places throughout this specification is not necessarily intended to refer to the same embodiment or to any one particular embodiment described. Further, it is to be understood that particular features, structures, or characteristics described may be combined in various ways in one or more embodiments. In general, of course, these and other issues may vary with the particular context of usage. Therefore, the particular context of the description or the usage of these terms may provide helpful guidance regarding inferences to be drawn for that context.

Although the subject matter has been described in language specific to structural features and/or methodological acts, it is to be understood that the subject matter defined in the appended claims is not necessarily limited to the specific features or acts described. Rather, the specific features and acts are disclosed as illustrative forms of implementing the claims.

One skilled in the art will realize that a virtually unlimited number of variations to the above descriptions are possible, and that the examples and the accompanying figures are merely to illustrate one or more examples of implementations.

It will be understood by those skilled in the art that various other modifications may be made, and equivalents may be substituted, without departing from claimed subject matter. Additionally, many modifications may be made to adapt a particular situation to the teachings of claimed subject matter without departing from the central concept described herein. Therefore, it is intended that claimed subject matter is not limited to the particular embodiments disclosed, but that such claimed subject matter may also include all embodiments falling within the scope of the appended claims, and equivalents thereof.

In the detailed description above, numerous specific details are set forth to provide a thorough understanding of claimed subject matter. However, it will be understood by those skilled in the art that claimed subject matter may be practiced without these specific details. In other instances, methods, apparatuses, or systems that would be known by one of ordinary skill have not been described in detail so as not to obscure claimed subject matter.

What is claimed is:

1. A skin cleansing article configured to deliver cosmetics or dermal therapies to a user's skin, the skin cleansing article comprising:
a core substrate comprising a resin, the core substrate having a first region containing a first active cleansing formulation and a second region containing a second active cleansing formulation,
wherein the skin cleansing article is configured to be at least water-dispersible or water-soluble, and the core substrate is at least dispersible or soluble upon contact with water having a temperature greater than 10° C. for a period of time according to Testing Method MSTM-205 so as to release at least one of the first active cleansing formulation and the second active cleansing formulation.

2. The skin cleansing article according to claim 1, wherein the skin cleansing article is substantially dry or solid with a content of moisture or solvent less than 10 wt. % before contacting with water.

3. The skin cleansing article according to claim 1, wherein the period of time is in a range of from about 30 seconds to about 300 seconds, or from about 30 seconds to about 600 seconds, or from about 30 seconds to about 900 seconds.

4. The skin cleansing article according to claim 1, wherein, at a temperature of 30° C. to 40° C., the core substrate has a dispersion time or a dissolution time of 300 seconds or less according to MSTM-205.

5. The skin cleansing article according to claim 1, wherein the core substrate is substantially planar and formable to a contour of a surface of the user's skin.

6. The skin cleansing article according to claim 1, wherein the core substrate comprises at least one nonwoven substrate including a plurality of fibers comprising the resin selected from at least one of a water-dispersible resin or a water-soluble resin.

7. The skin cleansing article according to claim 6, wherein the resin is a polymer comprising a vinyl alcohol moiety.

8. The skin cleansing article according to claim 7, wherein the vinyl alcohol moiety comprises a polyvinyl alcohol homopolymer, a polyvinyl alcohol copolymer, or a combination thereof.

9. The skin cleansing article according to claim 8, wherein the polyvinyl alcohol copolymer is a copolymer of vinyl acetate and vinyl alcohol.

10. The skin cleansing article according to claim 8, wherein the polyvinyl alcohol copolymer comprises an anionically modified copolymer.

11. The cleansing article according to claim 10, wherein the anionically modified copolymer comprises a carboxylate, a sulfonate, or combinations thereof.

12. The cleansing article according to claim 8, wherein the plurality of fibers comprise a first type of fiber comprising a polyvinyl alcohol copolymer having a degree of hydrolysis in a range of about 75% to about 89%, and a second type of fiber comprising a polyvinyl alcohol copolymer having a degree of hydrolysis in a range of about 90% to about 99.5%.

13. The cleansing article according to claim 12, wherein a ratio of the first type of fiber to the second type of fiber is in a range of from about 25:75 to about 75:25 by weight.

14. The cleansing article according to claim 12, wherein the first type of fiber and the second type of fiber are mixed together in the at least one nonwoven sheet.

15. The cleansing article according to claim 14, wherein the at least one nonwoven sheet comprises a first nonwoven layer made of the first type of fiber and a second nonwoven layer made of the second type of fiber.

16. The skin cleansing article according to claim 6, wherein the plurality of fibers comprise 10% to 80% water-soluble fibers by weight based on a total weight of the plurality of fibers and a balance being non-water-soluble fibers.

17. The skin cleansing article according to claim 1, wherein the first region is positionable at a first location on a face of the user and the second region is positionable at a second location on the face of the user.

18. The skin cleansing article according to claim 8, wherein the first region is separable from the second region.

19. The skin cleansing article according to claim 1, wherein each of the first active cleansing formulation and the second active cleansing formulation is in the form of at least one of the following: a solid, a liquid, a gel, or a slurry form.

20. The skin cleansing article according to claim 1, wherein at least one of the first active cleansing formulation or the second active cleansing formulation is in the solid form having a moisture content of less than 10%.

21. The skin cleansing article according to claim 1, wherein each of the first active cleansing formulation and the second active cleansing formulation comprises one or more of the following: hyaluronic acid, aloe, chamomile extract, lactic acid, citric acid, hydrolyzed collagen, a poly saccharide, a peptide, a surfactant, a foaming agent, ceramides, glycolic acid, an alpha-hydroxy acid, an amino acid, a activated carbon, sunscreen, minerals (Zn), avobenzone, antioxidants, energizers, caffeine, ginsing, taurine, retinol, retinoic acid, Niacinamide, salicyclic acid, lactic acid, or aseliaic acid, or any combination thereof.

22. The skin cleansing article according to claim 1, wherein each of the first active cleansing formulation and the second active cleansing formulation is at least one of disposed on a surface of the core substrate or embedded in a matrix of the core substrate.

23. The skin cleansing article according to claim 1, wherein the core substrate is at least one of saturated with an active cleansing formulation, coated with an active cleansing formulation, or impregnated with an active cleansing formulation.

24. The skin cleansing article according to claim 1, wherein the core substrate transitions into a hydrogel upon contact with water for 300 seconds or less at 10° C. or above.

25. A facial mask configured to deliver cosmetics or dermal therapies to a user's skin, the facial mask, comprising:
a nonwoven substrate including a plurality of fibers comprising a water-soluble resin, the nonwoven substrate being water-soluble and having a first region and a second region;
a first active cleansing formulation contained in the first region; and
a second active cleansing formulation contained in the second region,
wherein the nonwoven substrate is soluble upon contact with water having a temperature greater than 10° C. for 300 seconds or less according to Testing Method MSTM-205 so as to release at least one of the first active cleansing formulation and the second active cleansing formulation from the water-soluble nonwoven substrate.

26. The facial mask according to claim 25, wherein, at a temperature of 30° C. to 40° C., the nonwoven substrate has a dissolution time of 300 seconds or less according to MSTM-205.

27. The facial mask according to claim 25, wherein the nonwoven substrate has a moisture content of less than 10%.

28. The facial mask according to claim 25, wherein the nonwoven substrate is substantially planar and formable to a contour of a surface of the user's skin.

29. The facial mask according to claim 25, wherein the first region is positionable at a first location on a face of the user and the second region is positionable at a second location on the face of the user, wherein the first region is separate from the second region.

30. The mask according to claim 25, wherein each of the first active cleansing formulation and the second active cleansing formulation is in the form of at least one of the following: a solid, a liquid, a gel, or a slurry form.

31. The facial mask according to claim 25, wherein each of the first active cleansing formulation and the second active cleansing formulation comprises one or more of the following: hyaluronic acid, aloe, chamomile extract, lactic acid, citric acid, hydrolyzed collagen, a poly saccharide, a peptide, a surfactant, a foaming agent, ceramides, glycolic acid, an alpha-hydroxy acid, an amino acid, a activated carbon, sunscreen, minerals (Zn), avobenzone, antioxidants, energizers, caffeine, ginsing, taurine, retinol, retinoic acid, Niacinamide, salicyclic acid, lactic acid, or aseliaic acid, or any combination thereof.

32. The facial mask according to claim 25, wherein the plurality of fibers are saturated with one of the first active cleansing formulation or the second active cleansing formulation.

33. The facial mask according to claim 25, wherein each of the first active cleansing formulation and the second active cleansing formulation is one of disposed on a surface of the plurality of fibers or embedded in the plurality of fibers.

34. The facial mask according to claim 25, wherein the plurality of fibers comprise a fiber type including one or more of the following: a polyvinyl alcohol homopolymer, a polyvinyl alcohol copolymer, or a combination thereof.

35. The facial mask according to claim 34, wherein the polyvinyl alcohol copolymer has a degree of hydrolysis in a range of about 75% to about 89%.

36. The facial mask according to claim 34, wherein the polyvinyl alcohol copolymer has a degree of hydrolysis in a range of about 90% to about 99.9%.

37. The facial mask according to claim 34, wherein the polyvinyl alcohol copolymer comprises an anionically modified copolymer comprising a carboxylate, a sulfonate, or combinations thereof.

38. The facial mask according to claim 25, wherein the plurality of fibers comprise a first type of fiber and a second type of fiber, wherein the first type of fiber and the second type of fiber have a difference in one or more of the following characteristics: a length to diameter (LID) ratio, a tenacity, a shape, a rigidness, an elasticity, a solubility in water, amelting point, a glass transition temperature (T9), a fiber chemical composition, or a color, or any combination thereof.

39. The facial mask according to claim 38, wherein each of the first type of fiber and the second type of fiber comprises a polyvinyl alcohol homopolymer, a polyvinyl alcohol copolymer, or a combination thereof.

40. The facial mask according to claim 38, wherein the first type of fiber comprises a polyvinyl alcohol copolymer having a degree of hydrolysis in a range of 75% to 89%, and
the second type of fiber comprises a polyvinyl alcohol copolymer having a degree of hydrolysis in a range of 90% to 99.5%.

41. The facial mask according to claim 40, wherein a ratio of the first type of fiber to the second type of fiber is in a range of from about 25:75 to about 75:25 by weight.

42. The facial mask according to claim 40, wherein the first type of fiber and the second type of fiber are mixed together in a same nonwoven sheet or separated in different nonwoven sheets.

43. The facial mask according to claim 38, wherein one of the first type of fiber or the second type of fiber comprises a non-water-soluble polymer fiber forming material.

44. The facial mask according to claim 43, wherein the non-water-soluble polymer fiber-forming material comprises one or more of the following materials: cotton, hemp, jute, flax, ramie, sisal, bagasse, banana, lacebark, silk, sinew, catgut, wool, sea silk, mohair, angora, cashmere, collagen, actin, nylon, dacron, rayon, bamboo, modal, cellulose diacetate, cellulose triacetate, or combinations thereof.

45. The facial mask according to claim 25, wherein the water-soluble nonwoven substrate has a linear mass density in a range of 1 dtex to 5 dtex.

46. The facial mask according to claim 25, wherein the water-soluble nonwoven substrate is biodegradable.

47. The facial mask according to claim 25, wherein the plurality of fibers comprise at least one fiber type having a tenacity in a range of 3 cN/dtex to 15 cN/dtex,
having a mean diameter in a range of 10 microns to 300 microns,
having substantially uniform mean diameters,
and/or having an average length in a range of 10 millimeters (mm) to 100 mm.

48. The facial mask according to claim 25, wherein the nonwoven substrate has a porosity of 30% to 90%.

49. The facial mask according to claim 27, wherein the water-soluble resin comprises a polyvinyl alcohol copolymer having a degree of hydrolysis in a range of 75% to 99.9%.

50. The facial mask according to claim 25, wherein upon contact with water having a temperature greater than 10° C., the nonwoven substrate exhibits a degree of shrinkage of 0.5% to 65%.

51. A method for making a skin cleansing article, the method comprising:
forming a core substrate comprising a water-soluble resin, the water-soluble nonwoven substrate having a first region and a second region; and
containing a first active cleansing formulation in the first region and containing a second active cleansing formulation in the second region.

52. The method according to claim 51, wherein containing a first active cleansing formulation in the first region comprises at least one of saturating the first region of the core substrate with the first active cleansing formulation, disposing the first active cleansing formulation on a surface of the first region of the core substrate, coating a surface of the first region of the core substrate with the first active cleansing formulation, embedding the first active cleansing formulation in the first region of the core substrate, or impregnating the first region of the core substrate with the first active cleansing formulation.

53. The method according to claim 51, wherein forming the core substrate comprising a resin comprises recycling the core substrate to produce the resin.

54. The method according to claim 51, wherein the resin and the core substrate are water-soluble, and the skin cleansing article is a water-soluble facial mask.

55. A skin cleansing article configured to deliver cosmetics or dermal therapies to a user's skin, the skin cleansing article comprising:
a core substrate comprising a resin, the core substrate having a first region containing a first active cleansing formulation, wherein the skin cleansing article is substantially dry or solid, and is configured to be at least water-dispersible or water-soluble,
wherein the core substrate is at least dispersible or soluble upon contact with water having a temperature greater than 10° C. for a period of time according to Testing Method MSTM-205 so as to release the first active cleansing formulation from the water-dispersible core substrate.

56. The skin cleansing article according to claim 55, wherein the temperature is greater than 40° C. and the period of time is from 30 seconds to 300 seconds.

57. A skin cleansing article configured to deliver cosmetics or dermal therapies to a user's skin, comprising:
a first nonwoven substrate including a plurality of fibers comprising a water-soluble resin or a water-dispersible resin, the first nonwoven substrate having a first region;
a first active cleansing formulation contained in the first region;
a second nonwoven substrate coupled to the first nonwoven substrate, the second nonwoven substrate including a plurality of fibers comprising one of a water-dispersible resin or a water-soluble resin, the second nonwoven substrate having a second region; and
a second active cleansing formulation contained in the second region,
wherein, when the first nonwoven substrate is contacted with water having a temperature greater than 10° C. for 300 seconds or less, the first nonwoven substrate is soluble or dispersible according to MSTM-205 to release the first active cleansing formulation from the first nonwoven substrate.

58. The skin cleansing article according to claim 57, wherein the first nonwoven substrate is water soluble, the second nonwoven substrate includes a plurality of fibers comprising a water-dispersible resin, and, when the second nonwoven substrate is contacted with water having a temperature greater than 10° C. for 300 seconds or less, the second nonwoven substrate is dispersible according to MSTM-205 to release the second active cleansing formulation from the second nonwoven substrate.

59. The skin cleansing article according to claim 57, wherein the first nonwoven is water soluble, the second nonwoven substrate includes a plurality of fibers comprising a water-soluble resin, and, when the second nonwoven substrate is contacted with water having a temperature greater than 10° C. for 300 seconds or less, the second nonwoven substrate is soluble according to MSTM-205 to release the second active cleansing formulation from the second nonwoven substrate.

60. The skin cleansing article according to claim 57, further comprising a water-soluble or water dispersible film coupled to one of the first nonwoven substrate and the second nonwoven substrate.
